(19) United States
(12) Patent Application Publication
(10) Pub. No.: US 2014/0073628 A1
(43) Pub. Date: Mar. 13, 2014

(54) BENZOXAZEPINES ASN INHIBITORS OF P13K/M TOR AND METHODS OF THEIR USE AND MANUFACTURE

(71) Applicant: Kenneth D. Rice, San Rafael, CA (US)

(72) Inventor: Kenneth D. Rice, San Rafael, CA (US)

(21) Appl. No.: 13/988,948

(22) PCT Filed: Nov. 23, 2011
(86) PCT No.: PCT/US2011/062025

§ 371 Date: Nov. 4, 2013

Related U.S. Application Data
(60) Provisional application No. 61/417,165, filed on Nov. 24, 2010.

(51) Int. Cl. C07D 413/14; C07D 413/04

(52) U.S. Cl. CPC C07D 413/14; C07D 413/04
USPC 514/210.21; 540/552; 514/211.09

(57) ABSTRACT

The invention is directed to inhibitors of mTOR and pharmaceutically acceptable salts or solvates thereof, as well as methods of using them. The inhibitors are generally of structural formula I and pharmaceutically acceptable salts thereof, wherein the variables are as defined herein.

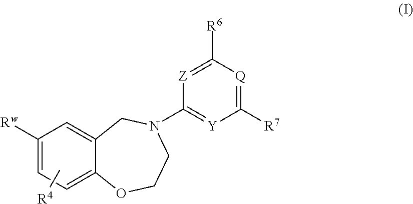

### CROSS-REFERENCE TO RELATED APPLICATIONS

This application claims the benefit of priority to U.S. Provisional Application No. 61/417,165, filed Nov. 24, 2010, which is incorporated herein by reference.

### SEQUENCE LISTING

This application incorporates by reference in its entirety the Sequence Listing entitled “10-024_Sequence.txt” (16.7 KB) which was created Nov. 23, 2011 and filed herewith on Nov. 23, 2011.

### BACKGROUND OF THE INVENTION

1. Field of the Invention

This invention relates to the field of protein kinases and inhibitors thereof. In particular, the invention relates to inhibitors of PI3K and/or mammalian target of rapamycin (mTOR) signaling pathways, and methods of their use and preparation.

2. Background of the Invention

The PI3K pathway regulates cell growth, proliferation and survival, and is dysregulated with high frequency in human tumors. PI3K pathway activation in tumors occurs via multiple mechanisms including prevalent mutation and amplification of the PIK3CA gene (which encodes the p110 subunit of PI3Ka), or downregulation of the lipid phosphatase PTEN. Downstream of PI3K, mTOR controls cell growth and proliferation through its two distinct signaling complexes: mTORC1 and mTORC2. Given the role of PI3K signaling on critical cellular functions, an inhibitor that targets both PI3K and mTOR could provide therapeutic benefit to patient populations with tumors harboring activating mutations in PIK3CA or Ras, PTEN-deletion, or where tumors are upregulated in growth factor signaling.

Recent studies indicate that phosphatidylinositol 3-kinase (PI3K) signaling has significant effects on cancer cell growth, survival, motility, and metabolism. The PI3K pathway is activated by several different mechanisms in cancers, including somatic mutation and amplification of genes encoding key components. In addition, PI3K signaling may serve integral functions for noncancerous cells in the tumor microenvironment. Consequently, there is continued interest in developing inhibitors of PI3K isoforms as a means for treating various forms of cancer, particularly the class II isoforms PI3K-alpha, PI3K-beta, and PI3k-gamma.

For example, Phosphatidylinositol 3-kinase-alpha (PI3Kα), a dual specificity protein kinase, is composed of an 85 kDa regulatory subunit and a 110 kDa catalytic subunit. The protein encoded by this gene represents the catalytic subunit, which uses ATP to phosphorylate PtdIns, PtdIns4P and PtdIns(4,5)P2. PTEN, a tumor suppressor which inhibits cell growth through multiple mechanisms, can dephosphorylate PIP3, the major product of PIK3CA. PIP3, in turn, is required for translocation of protein kinase B (AKT1, PKB) to the cell membrane, where it is phosphorylated and activated by upstream kinases. The effect of PTEN on cell death is mediated through the PIK3CA/AKT1 pathway.

PI3Kα has been implicated in the control of cytoskeletal reorganization, apoptosis, vesicular trafficking, proliferation and differentiation processes. Increased copy number and expression of PIK3CA is associated with a number of malignancies such as ovarian cancer (Campbell et al., Cancer Res 2004, 64, 7678-7681; Levine et al., Clin Cancer Res 2005, 11, 2875-2878; Wang et al., Hum Mutat 2005, 25, 322; Lee et al., Gynecol Oncol 2005, 97, 26-34), cervical cancer, breast cancer (Bachman, et al. Cancer Biol Ther 2004, 3, 772-775; Levine, et al., supra; Li et al., Breast Cancer Res Treat 2006, 96, 91-95; Saal et al., Cancer Res 2005, 65, 2554-2559; Samuels and Velculescu, Cell Cycle 2004, 3, 1221-1224), colorectal cancer (Samuels, et al. Science 2004, 304, 554; Velho et al. Eur J Cancer 2005, 41, 1649-1654), endometrial cancer (Oda et al. Cancer Res. 2005, 65, 10669-10673), gastric carcinomas (Byun et al., Int J Cancer 2003, 104, 318-327; Li et al., supra; Velho et al., supra; Lee et al., Oncogene 2005, 24, 1477-1480), hepatocellular carcinoma (Lee et al., id.), small and non-small cell lung cancer (Tang et al., Lung Cancer 2006, 51, 181-191; Massion et al., Am J Respir Crit. Care Med 2004, 170, 1088-1094), thyroid carcinoma (Wu et al., J Clin Endocrinol Metab 2005, 90, 4688-4693), acute myelogenous leukemia (AML) (Sujobert et al., Blood 1997, 106, 1063-1066), chronic myelogenous leukemia (CML) (Hickey and Cotter J Biol Chem 2006, 281, 2441-2450), and glioblastomas (Hartmann et al. Acta Neuropathol (Berl) 2005, 109, 639-642; Samuels et al., supra).

The mammalian target, mTOR, is a protein kinase that integrates both extracellular and intracellular signals of cellular growth, proliferation, and survival. Extracellular mitogenic growth factor signaling from cell surface receptors and intracellular pathways that convey hypoxic stress, energy and nutrient status all converge at mTOR. mTOR exists in two distinct complexes: mTOR complex 1 (mTORC1) and mTOR complex 2 (mTORC2). mTORC1 is a key mediator of transcription and cell growth (via its substrates p70S6 kinase and 4E-BP1) and promotes cell survival via the serum and glucocorticoid-activated kinase SGK, whereas mTORC2 promotes activation of the pro-survival kinase AKT. Given its central role in cellular growth, proliferation and survival, it is perhaps not surprising that mTOR signaling is frequently dysregulated in cancer and other diseases (Bjornsti and Houghton Rev Cancer 2004, 4(5), 335-48; Houghton and Huang Microbiol Immunol 2004, 279, 339-59; Inoki, Corradetti et al. Nat Genet. 2005, 37(1), 19-24).

mTOR is a member of the PIKK (PI3K-related Kinase) family of atypical kinases which includes ATM, ATR, and DNAPK, and its catalytic domain is homologous to that of PI3K. Dyregulation of PI3K signaling is a common function of tumor cells. In general, mTOR inhibition may be considered as a strategy in many of the tumor types in which PI3K signaling is implicated such as those discussed below.

Inhibitors of mTOR may be useful in treating a number of cancers, including the following: breast cancer (Nagata, Lan et al., Cancer Cell 2004, 6(2), 117-27; Pandolfi N Engl J Med 2004, 351(22), 2337-8; Nahta, Yu et al. Nat Clin Pract Oncol 2006, 3(5), 269-280); antle cell lymphoma (MCL) (Dal Col, Zancai et al. Blood 2008, 111(10), 5142-51); renal cell carcinoma (Thomas, Tran et al. Nat Med 2006, 12(1), 122-7; Atkins, Hidalgo et al. J Clin Oncol 2004, 22(5), 909-18; Motzer, Hudes et al. J Clin Oncol 2007, 25(25), 3958-64); acute myelogenous leukemia (AML) (Sujobert, Bardet et al. Blood 2005, 106(3), 1063-6; Billottet, Grandage et al. Oncogene 2006, 25(50), 6648-6659; Tamburini, Elie et al. Blood 2007, 110(3), 1025-8); chronic myelogenous leukemia (CML) (Skorski, Bellacosa et al. Embo J 1997, 16(20), 6151-61; Bai, Ouyang et al. Blood 2000, 96(13), 4319-27; Hickey and Cotter Biol Chem 2006, 281(5), 2441-50); diffuse large B cell lymphoma (DLBCL) (Uddin, Hussain et al. Blood 2006, 108(13), 4178-86); several subtypes of sarcoma (Hernando, Charytonowicz et al. Nat Med 2007, 13(6), 748-53; Wan and Helman Oncologist 2007, 12(8), 1007-18); rhabdomyosarcoma (Cao, Yu et al. Cancer Res 2008, 68(19), 8039-8048; Wan, Shen et al. Neoplasia 2006, 8(5), 394-401); ovarian cancer (Shayesteh, Lu et al. Nat Genet, 1999, 21(1), 99-102; (Lee, Choi et al. Gynecol Oncol 2005, 97(1) 26-34); endometrial tumors (Obata, Morland et al. Cancer Res 1998, 58(10), 2095-7; Lu, Wu et al. Clin Cancer Res 2008, 14(9), 2543-50); non small cell lung carcinoma (NSCLC) (Tang, He et al. Lung Cancer 2006, 51(2), 181-91; Marsit, Zheng et al. Hum Pathol 2005, 36(7), 768-76); small cell, squamous, large cell and adenocarcinoma (Massion, Taflan et al. Am J Respir Crit. Care Med 2004, 170(10), 1088-94); lung tumors in general (Kokubo, Gemma et al. Br J Cancer 2005, 92(9), 1711-9; Pao, Wang et al. Pub Library of Science Med 2005, 2(1), e17); colorectal tumors (Velho, Oliveira et al. Eur J Cancer 2005, 41(11), 1649-54; Foukas, Claret et al. Nature, 2006, 441(7091), 366-370), particularly those that display microsatellite instability (Goel, Arnold et al. Cancer Res 2004, 64(9), 3014-21; Nassif, Lobo et al. Oncogene 2004, 23(2), 617-28), KRAS-mutated colorectal tumors (Bos Cancer Res 1989. 49(17), 4682-9; Fearon Ann N Y Acad Sci 1995, 768, 101-10); gastric carcinomas (Byun, Cho et al. Int J Cancer 2003, 104(3), 318-27); hepatocellular tumors (Lee, Soung et al. Oncogene 2005, 24(8), 1477-80); liver tumors (Hu, Huang et al. Cancer 2003, 97(8), 1929-40; Wan, Jiang et al. Cancer Res Clin Oncol 2003, 129(2), 100-6); primary melanomas and associated increased tumor thickness (Guldberg, thor Straten et al. Cancer Res 1997, 57(17), 3660-3; Tsao, Zhang et al. Cancer Res 2000, 60(7), 1800-4; Whiteman, Zhou et al. Int J Cancer 2002, 99(1), 63-7; Goel, Lazar et al. J Invest Dermatol 126(1), 2006, 154-60); pancreatic tumors (Asano, Yao et al. Oncogene 2004, 23(53), 8571-80); prostate carcinoma (Cairns, Okami et al. Cancer Res 1997, 57(22), 4997-5000; Gray, Stewart et al. Br J Cancer 1998, 78(10), 1296-300; Wang, Parsons et al. Clin Cancer Res 1998, 4(3), 811-5; Whang, Wu et al. Proc Natl Acad Sci USA 1998, 95(9), 5246-50; Majumder and Sellers Oncogene 2005, 24(50) 7465-74; Wang, Garcia et al. Proc Natl Acad Sci USA 2006, 103(5), 1480-5; (Lu, Ren et al. Int J Oncol 2006, 28(1), 245-51; Mulholland, Dedhar et al. Oncogene 25(3), 2006, 329-37; Xin, Teitell et al. Proc Natl Acad Sci USA 12006, 03(20), 7789-94; Mikhailova, Wang et al. Adv Exp Med Biol 2008, 617, 397-405; Wang, Mikhailova et al. Oncogene 2008, 27(56), 7106-7117); thyroid carcinoma, particularly in the anaplastic subtype (Garcia-Rostan, Costa et al. Cancer Res 2005, 65(22), 10199-207); follicular thyroid carcinoma (Wu, Mambo et al. J Clin Endocrinol Metab 2005, 90(8), 4688-93); anaplastic large cell lymphoma (ALCL); hamaratomas, angiomyelolipomas, TSC-associated and sporadic lymphangioleiomyomatosis: Cowden's disease (multiple hamaratoma syndrome) (Bissler, McCormack et al. N Engl J Med 2008, 358(2), 140-151); sclerosing hemangioma (Randa M. S. Amin Pathology International 2008, 58(1), 38-44); Peutz-Jeghers syndrome (PJS); head and neck cancer (Gupta, McKenna et al. Clin Cancer Res 2002, 8(3), 885-892); neurofibromatosis (Ferner Eur J Hum Genet. 2006, 15(2), 131-138; Sabatini Nat Rev Cancer 2006, 6(9), 729-734; Johannessen, Johnson et al. Current Biology 2008, 18(1), 56-62); macular degeneration; macular edema; myeloid leukemia; systemic lupus; and autoimmune lymphoproliferative syndrome (ALPS).

### SUMMARY OF THE INVENTION

The following only summarizes certain aspects of the invention and is not intended to be limiting in nature. These aspects and other aspects and embodiments are described more fully below. All references cited in this specification are hereby incorporated by reference in their entirety. In the event of a discrepancy between the express disclosure of this specification and the references incorporated by reference, the express disclosure of this specification shall control.

We recognized the important role of PI3K and mTOR in biological processes and disease states and, therefore, realized that inhibitors of these protein kinases would be desirable, as evidenced in Serial Number PCT/US2010/036032, filed May 25, 2010, the entire contents of which is incorporated herein by reference. Accordingly, the invention provides compounds that inhibit, regulate, and/or modulate PI3K and/or mTOR and are useful in the treatment of hyperproliferative diseases, such as cancer, in mammals. This invention also provides methods of making the compound, methods of using such compounds in the treatment of hyperproliferative diseases in mammals, especially humans, and to pharmaceutical compositions containing such compounds.

A first aspect of the invention provides a compound of formula I

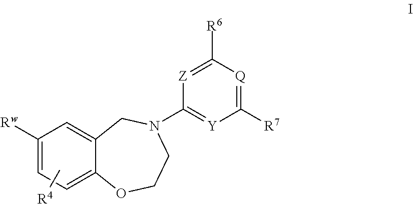

or a single isomer or mixture of stereoisomers thereof, optionally as a pharmaceutically acceptable salt thereof, wherein

R^{W} is

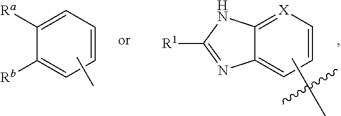

wherein:

R^{a} and R^{b} are each independently —CO(C₁-C₆)alkyl, —CO₂(C₁-C₆)alkyl, —SO₂—(C₁-C₆)alkyl, —NHSO₂—(C₁-C₆)alkyl, O—(C₁-C₆)alkyl, O—(C₁-C₆)haloalkyl, O—(C₁-C₆)alkylene-OR′″, wherein R′″ is H or (C₁-C₆)alkyl

R¹ is H, (C₁-C₆)alkyl), NH₂, NH(C₁-C₆)alkyl, halo(C₁-C₆)alkyl, (C₃-C₇)-cycloalkyl, NHCO₂(C₁-C₆)alkyl, or N((C₁-C₆)alkyl)₂;

X is N or CH;

R⁴ is H or halo;

Q is N, C—H, or C—(C₁-C₆)alkyl, C—CN, or C—CF₃;

R⁶ is H, (C₁-C₆)alkyl, halo(C₁-C₆)alkyl, (C₁-C₆)alkylene-NH₂, (C₁-C₆)alkylene-NH(C₁-C₆)alkyl, (C₁-C₆)alkylene-NH(C₁-C₆)haloalkyl, (C₁-C₆)alkylene-N(C₁-C₆)alkyl)₂, NH₂, NH(C₁-C₆)alkyl, hydroxyalkyl, (C₁-C₆)alkylene-O(C₁-C₆)alkyl, NH(C₁-C₆)alkyleneNH₂, NH(C₁-C₆)alkylene-cycloalkyl, —NH(C₁-C₆)alkylene-heterocycloalkyl, N((C₁-C₆)alkyl)₂, (C₁-C₆)alkylene-NHSO₂—(C₁-C₆)alkyl, (C₁-C₆)alkylene-NH(C═O)—(C₁-C₆)alkyl, —(C═O)—NH₂, —(C═O)—(C₁-C₆)alkyl, —(C═O)—NH(C₁-C₆)alkyl, —(C═O)—N(C₁-C₆)alkyl))₂, —NHSO₂—(C₁-C₆)alkyl, —S(O)—(C₁-C₆)alkyl, —SO₂—(C₁-C₆)alkyl, —SO₂NH₂, —SO₂NH(C₁-C₆)alkyl, —SO₂N((C₁-C₆)alkyl)₂, —CN, (C₄-C₇)heterocycloalkyl, (C₁-C₆)alkylene-(C₃-C₇)heterocycloalkyl, nitro, (C₁-C₆)alkylene-CN, NH(C₁-C₆)alkylene-NH(C₁-C₆)alkyl, NH(C₁-C₆)alkylene-N((C₁-C₆)alkyl)₂, or (C₁-C₆)alkylene-OC(O)—(C₁-C₆)alkyl, where any alkylene in R⁶ is optionally substituted with 1, 2, or 3 groups which are independently halo or hydroxy, and wherein when any alkylene is —CH₂—, then one of the hydrogens of the —CH₂— can optionally be replaced by (C1-C3)haloalkyl;

R⁷ is H, halo, —NH₂, nitro (C₁-C₆)alkyl, (C₁-C₆)alkoxy, R⁷ is —CF₃, halo(C₁-C₆)alkyl, (C₁-C₆)alkenyl, NH(C₁-C₆)alkyl, or N((C₁-C₆)alkyl)₂;

Y is N or C—R⁸, wherein R⁸ is H, halo, (C₁-C₆)alkyl, NH₂, NH(C₁-C₆)alkyl, N((C₁-C₆)alkyl)₂, (C₂-C₆)alkenyl, (C₁-C₆)alkylene-O(C₁-C₆)alkyl, hydroxyalkyl, (C₁-C₆)alkylene-CO₂(C₁-C₆)alkyl, (C₁-C₆)alkylene-CO₂H, phenyl, —CF₃, halo(C₁-C₆)alkyl, (C₃-C₇)cycloalkyl, (C₁-C₆)alkylene-(C₃-C₇)cycloalkyl, COH, CO₂H, —CO₂(C₁-C₆)alkyl, CN, (C₁-C₆)alkylene-CN, (C₁-C₆)alkylene-C≡C—H, (C₁-C₆)alkylene-C≡C—(C₁-C₆)alkyl, —C≡C—H, —C≡C—(C₁-C₆)alkyl, (C₁-C₆)alkylene-phenyl; where any phenyl in R⁸ is optionally substituted with 1, 2, or 3 groups which groups are independently halo or alkyl; or

R⁷ and R⁸, together with the atoms to which they are attached, can be joined together to form a 5, 6, or 7 membered saturated, partially unsaturated, or unsaturated ring, optionally containing up to two heteroatoms selected from N—H, N—(C₁-C₆)alkyl, O, SO, and SO₂ and where the ring formed by R⁷ and R⁸ is optionally substituted with one or two groups which are independently alkyl, alkoxy, or halo; and

Z is N or C—R⁹, wherein R⁹ is H, halo, or (C₁-C₆)alkyl; and

where at least one of Q and Z is N.

Embodiments of this aspect may include one or more of the following. The compound is a compound of formula Ia or 1-2.

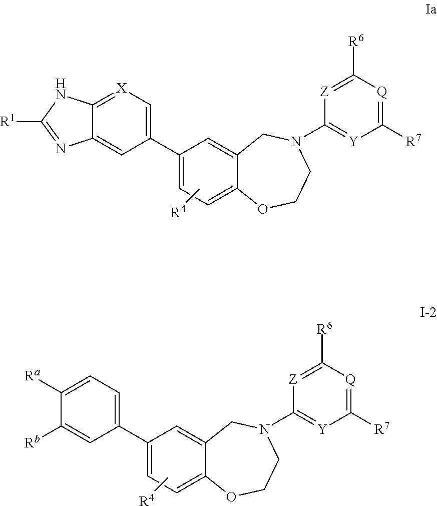

The compound is a compound of formula Ia.

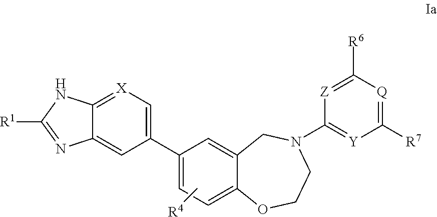

The compound is a compound of formula Ib.

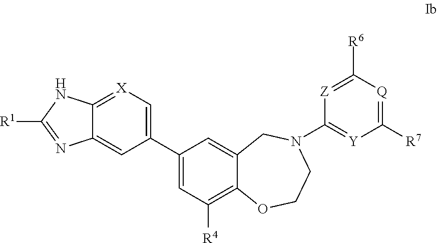

The compound is a compound of formula Ic-1 or Ic-2.

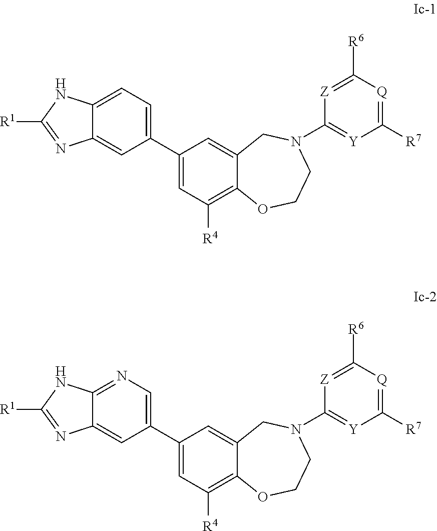

The compound is a compound of formula Id, wherein R⁴ is H or F.

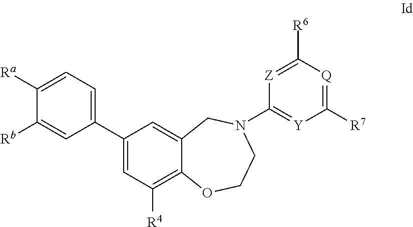

The compound is a compound of formula Ie.

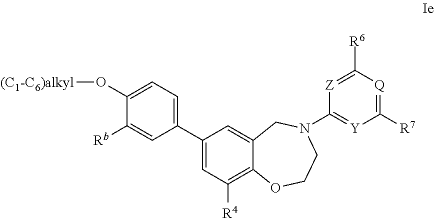

The compound is a compound of formula I wherein R⁴ is H.

In another aspect, the invention provides a compound of formula I or of Table 1, or a single stereoisomer or mixture of isomers thereof, optionally as a pharmaceutically acceptable salt or solvate thereof and 2) a pharmaceutically acceptable carrier, excipient, or diluent.

In another aspect, the invention provides a pharmaceutical composition comprising a compound of formula I or of Table 1, or a single stereoisomer or mixture of isomers thereof, optionally as a pharmaceutically acceptable salt or solvate thereof; and 2) a pharmaceutically acceptable carrier, excipient, or diluent.

In another aspect of the invention is a method of inhibiting the in vivo activity of mTOR, the method comprising administering to a patient an effective PI3K/mTOR-inhibiting amount of a compound of formula I or of Table 1 or a single stereoisomer or mixture of isomers thereof, optionally as a pharmaceutically acceptable salt or solvate thereof or pharmaceutical composition thereof. In this and other aspects and embodiments as provided herein, the compound can be an inhibitor of PI3Kα, PI3Kβ, PI3Kγ, or other PI3K isoforms combinations thereof.

In another aspect, the Invention provides a method for treating a disease, disorder, or syndrome which method comprises administering to a patient a therapeutically effective amount of a compound of formula I or a single stereoisomer or mixture of isomers thereof, optionally as a pharmaceutically acceptable salt or solvate thereof, or a pharmaceutical composition comprising a therapeutically effective amount of a compound of formula I or of Table 1 or a single stereoisomer or mixture of isomers thereof, optionally as a pharmaceutically acceptable salt or solvate thereof, and a pharmaceutically acceptable carrier, excipient, or diluent.

In an additional aspect of the invention provides a method for treating a subject having a tumor the method comprising: (a) administering a PI3K-α selective inhibitor, a dual PI3K-α/mTOR selective inhibitor, or a combination of a PI3K-α selective inhibitor and a mTOR selective inhibitor to the subject if said tumor comprises a mutation in a PI3K-α kinase domain; or (b) administering a combination of a PI3K-α selective inhibitor and a PI3K-β selective inhibitor, a dual PI3K-α/mTOR selective inhibitor, or a PI3K-β selective inhibitor, to said subject if said tumor comprises a mutation in a PI3K-α helical domain, wherein the PI3K-α selective inhibitor, the dual PI3K-α/mTOR selective inhibitor, or the combination of the PI3K-α selective inhibitor and a mTOR selective inhibitor is a compound of Formula I or of Table 1.

In an additional aspect, the present invention provides a method for identifying a selective inhibitor of a PI3K isozyme, the method comprising: (a) contacting a first cell bearing a first mutation in a PI3K-α with a candidate inhibitor; (b) contacting a second cell bearing a wild type PI3K-α, a PTEN null mutation, or a second mutation in said PI3K-α with the candidate inhibitor; and (c) measuring AKT phosphorylation in said first and said second cells, wherein decreased AKT phosphorylation in said first cell when compared to said second cell identifies said candidate inhibitor as a selective PI3K-α inhibitor, wherein the PI3K-α selective inhibitor, the dual PI3K-α/mTOR selective inhibitor, or the combination of the PI3K-α selective inhibitor and a mTOR selective inhibitor is a compound of Formula I or of Table 1.

In an additional aspect, the present invention provides for a method for determining a treatment regimen for a cancer patient having a tumor comprising a PI3K-α, the method comprising: determining the presence or absence of a mutation in amino acids 1047 and/or 545 of said PI3K-α; wherein if said PI3K-α has a mutation at position 1047, said method comprises administering to the cancer patient a therapeutically effective amount of a PI3K-α selective inhibitor compound, or a dual PI3K-α/mTOR selective inhibitor, or a combination of a PI3K-α selective inhibitor and a mTOR selective inhibitor; or wherein if said PI3K-α has a mutation at position 545, said method comprises administering to the cancer patient a therapeutically effective amount of a combination of a PI3K-α selective inhibitor and a PI3K-β selective inhibitor, or a dual PI3K-α/mTOR selective inhibitor, or a combination of a PI3K-α selective inhibitor and a mTOR selective inhibitor; wherein the PI3K-α selective inhibitor, the dual PI3K-α/mTOR selective inhibitor, or the combination of the PI3K-α selective inhibitor and a mTOR selective inhibitor is a compound of Formula I or of Table 1.

In an additional aspect, the cell used to diagnose, treat or screen against includes a cancer or tumor cell obtained from a tumor or cancer derived from: breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non-small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer, wherein the PI3K-α selective inhibitor, the dual PI3K-α/mTOR selective inhibitor, or the combination of the PI3K-α selective inhibitor and a mTOR selective inhibitor is a compound of Formula I or of Table 1.

### DETAILED DESCRIPTION OF THE INVENTION

### Abbreviations and Definitions

The following abbreviations and terms have the indicated meanings throughout:

| Abbreviation | Meaning |
|---|---|
| br | broad |
| ° C. | degrees Celsius |
| d | doublet |
| dd | doublet of doublet |
| dt | doublet of triplet |
| DCM | dichloromethane |
| DIEA or DIPEA | N,N-di-isopropyl-N-ethylamine |
| DMA | N,N-dimethylacetamide |
| DME | 1,2-dimethoxyethane |
| DMF | N,N-dimethylformamide |
| DMSO | dimethyl sulfoxide |
| dppf | 1,1′-bis(diphenylphosphano)ferrocene |
| EI | Electron Impact ionization |
| g | gram(s) |
| GC/MS | gas chromatography/mass spectrometry |
| h or hr | hour(s) |
| HPLC | high pressure liquid chromatography |
| L | liter(s) |
| LC/MS | liquid chromatography/mass spectrometry |
| M | molar or molarity |
| m | Multiplet |
| MeOH | methanol |
| mg | milligram(s) |
| MHz | megahertz (frequency) |
| min | minute(s) |
| mL | milliliter(s) |
| μL | microliter(s) |
| μM | micromolar |
| μmol | micromole(s) |
| mM | Millimolar |
| mmol | millimole(s) |
| mol | mole(s) |
| MS | mass spectral analysis |
| N | normal or normality |
| nM | nanomolar |
| NMP | N-methyl-2-pyrrolidone |
| NMR | nuclear magnetic resonance spectroscopy |
| q | Quartet |
| rt | Room temperature |
| s | Singlet |
| t or tr | Triplet |
| THF | tetrahydrofuran |

The symbol “—” means a single bond, “═” means a double bond, “=≡” means a triple bond, “ [symbol] ” means a single or double bond. The symbol “ [symbol] ” refers to a group on a double-bond as occupying either position on the terminus of a double bond to which the symbol is attached; that is, the geometry, E- or Z-, of the double bond is ambiguous. When a group is depicted removed from its parent Formula, the “ [symbol] ” symbol will be used at the end of the bond which was theoretically cleaved in order to separate the group from its parent structural Formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual Formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

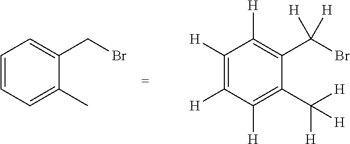

If a group “R” is depicted as “floating” on a ring system, as for example in the Formula:

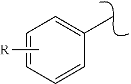

then, unless otherwise defined, a substituent “R” may reside on any atom of the ring system, assuming replacement of a depicted, implied, or expressly defined hydrogen from one of the ring atoms, so long as a stable structure is formed.

When a group “R” is depicted as existing on a ring system containing saturated carbons, as for example in the Formula:

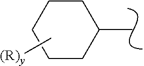

where, in this example, “y” can be more than one, assuming each replaces a currently depicted, implied, or expressly defined hydrogen on the ring; then, unless otherwise defined, where the resulting structure is stable, two “R's” may reside on the same carbon. In another example, two R's on the same carbon, including that carbon, may form a ring, thus creating a spirocyclic ring structure with the depicted ring as for example in the Formula:

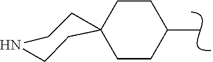

“Administration” and variants thereof (e.g., “administering” a compound) in reference to a Compound of the invention means introducing the Compound or a prodrug of the Compound into the system of the animal in need of treatment. When a Compound of the invention or prodrug thereof is provided in combination with one or more other active agents (e.g., surgery, radiation, and chemotherapy, etc.), “administration” and its variants are each understood to include concurrent and sequential introduction of the Compound or prodrug thereof and other agents.

“Alkenyl” means a means a linear monovalent hydrocarbon radical of two to six carbon atoms or a branched monovalent hydrocarbon radical of three to six carbon atoms which radical contains at least one double bond, e.g., ethenyl, propenyl, 1-but-3-enyl, and 1-pent-3-enyl, and the like.

“Alkoxy” means an —OR group where R is alkyl group as defined herein. Examples include methoxy, ethoxy, propoxy, isopropoxy, and the like.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), or pentyl (including all isomeric forms), and the like.

“Alkylene” means a linear saturated monovalent hydrocarbon diradical of one to six carbon atoms or a branched saturated monovalent hydrocarbon radical of three to six carbon atoms, e.g., methylene, ethylene, propylene, and the like.

“Alkynyl” means a linear monovalent hydrocarbon radical of two to six carbon atoms or a branched monovalent hydrocarbon radical of three to 6 carbon atoms which radical contains at least one triple bond, e.g., ethynyl, propynyl, butynyl, pentyn-2-yl and the like.

“Amino” means —NH₂.

“Aryl” means a monovalent six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Representative examples include phenyl, naphthyl, and indanyl, and the like.

“Arylalkyl” means an alkyl radical, as defined herein, substituted with one or two aryl groups, as defined herein, e.g., benzyl and phenethyl, and the like.

“Cycloalkyl” means a monocyclic or fused bicyclic, saturated or partially unsaturated (but not aromatic), monovalent hydrocarbon radical of three to ten carbon ring atoms. Fused bicyclic hydrocarbon radical includes Spiro and bridged ring systems. Unless stated otherwise, the valency of the group may be located on any atom of any ring within the radical, valency rules permitting. One or two ring carbon atoms may be replaced by a —C(O)—, —C(S)—, or —C(═NH)— group. More specifically, the term cycloalkyl includes, but is not limited to, cyclopropyl, cyclobutyl, cyclopentyl, cyclohexyl, cyclohexyl, or cyclohex-3-enyl, and the like.

“Dialkylamino” means an —NRR′ radical where R and R′ are alkyl as defined herein, or an N-oxide derivative, or a protected derivative thereof, e.g., dimethylamino, diethylamino, N,N-methylpropylamino or N,N-methylethylamino, and the like.

“Fused ring system” means a polycyclic ring system that contains bridged or fused rings; that is, where two rings have more than one shared atom in their ring structures. In this application, fused ring systems are not necessarily all aromatic ring systems. Typically, but not necessarily, fused ring systems share a vicinal set of atoms, for example naphthalene or 1,2,3,4-tetrahydro-naphthalene. Fused ring systems of the invention may themselves have spiro rings attached thereto via a single ring atom of the fused ring system. In some examples, as appreciated by one of ordinary skill in the art, two adjacent groups on an aromatic system may be fused together to form a ring structure. The fused ring structure may contain heteroatoms and may be optionally substituted with one or more groups.

“Halogen” or “halo” refers to fluorine, chlorine, bromine and iodine.

“Halo(C₁-C₆)alkyl” and “(C₁-C₆)haloalkyl” mean an alkyl group substituted with one or more halogens, specifically 1, 2, 3, 4, 5, or 6 halo atoms, e.g., trifluoromethyl, 2-chloroethyl, and 2,2-difluoroethyl, and the like.

“Heteroaryl” means a monocyclic or fused bicyclic or tricyclic monovalent radical of 5 to 14 ring atoms containing one or more, specifically one, two, three, or four ring heteroatoms where each heteroatom is independently —O—, —S(O)ₙ— (n is 0, 1, or 2), —N═, —NH—, or N-oxide, with the remaining ring atoms being carbon, wherein the ring comprising a monocyclic radical is aromatic and wherein at least one of the fused rings comprising the bicyclic radical is aromatic. One or two ring carbon atoms of any nonaromatic rings comprising a bicyclic radical may be replaced by a —C(O)—, —C(S)—, or —C(═NH)— group. Fused bicyclic radical includes bridged ring systems. Unless stated otherwise, the valency may be located on any atom of any ring of the heteroaryl group, valency rules permitting. When the point of valency is located on the nitrogen, R^{x} is absent. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, 5,6,7,8-tetrahydroquinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, 5,6,7,8-tetrahydroquinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, 2,3-dihydrobenzofuranyl, benzo[d][1,3]dioxolyl, 2,3-dihydrobenzo[b][1,4]dioxinyl, thiazolyl, isothiazolyl, thiadiazolyl, benzothiazolyl, benzothienyl, 6,7-dihydro-5H-cyclopenta[b]pyridinyl, 6,7-dihydro-5H-cyclopenta[c]pyridinyl, 6,7-dihydro-5H-cyclopenta[d]pyrimidinyl, 5,6,7,8-tetrahydro-5,8-ethanoquinazolin-4-yl, and 6,7,8,9-tetrahydropyrimido[4,5-b]indolizin-4-yl, and the N-oxide thereof and a protected derivative thereof.

“Heterocycloalkyl” means a saturated or partially unsaturated (but not aromatic) monovalent monocyclic group of 3 to 8 ring atoms or a saturated or partially unsaturated (but not aromatic) monovalent fused or spirocyclic bicyclic group of 5 to 12 ring atoms in which one or more, specifically one, two, three, or four ring heteroatoms where each heteroatom is independently O, S(O)ₙ (n is 0, 1, or 2), —NH—, or —N═, the remaining ring atoms being carbon. One or two ring carbon atoms may be replaced by a —C(O)—, —C(S)—, or —C(═NH)— group. Fused bicyclic radical includes bridged ring systems. Unless otherwise stated, the valency of the group may be located on any atom of any ring within the radical, valency rules permitting. When the point of valency is located on a nitrogen atom, R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydrocyclopenta[c]pyrrolyl, octahydroindolyl, octahydroisoindolyl, decahydroisoquinolyl, 2,6-diazaspiro[3.3]heptan-2-yl, tetrahydrofuryl, and tetrahydropyranyl, and the derivatives thereof and N-oxide or a protected derivative thereof.

“Phenylalkyl” means an alkyl group, as defined herein, substituted with one or two phenyl groups.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term, unless stated otherwise. A list of exemplary optional substitutions is presented below in the definition of “substituted.”

“Oxo” means an oxygen which is attached via a double bond.

“Yield” for each of the reactions described herein is expressed as a percentage of the theoretical yield.

“Metabolite” refers to the break-down or end product of a Compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds), 1990 for a discussion of biotransformation). As used herein, the metabolite of a Compound of the invention or its salt may be the biologically active form of the Compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a Compound of the present invention is known to one of skill in the art in light of the present disclosure.

“Patient” for the purposes of the present invention includes humans and other animals, particularly mammals, and other organisms. Thus the methods are applicable to both human therapy and veterinary applications. In a specific embodiment the patient is a mammal, and in a more specific embodiment the patient is human.

A “pharmaceutically acceptable salt” of a Compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent Compound is replaced by a metal ion, such as sodium, potassium, lithium, ammonium, calcium, magnesium, iron, zinc, copper, manganese, aluminum salts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine. “Platin(s),” and “platin-containing agent(s)” include, for example, cisplatin, carboplatin, and oxaliplatin.

“Prodrug” refers to compounds that are transformed (typically rapidly) in vivo to yield the parent Compound of the above Formula e, for example, by hydrolysis in blood. Common examples include, but are not limited to, ester and amide forms of a Compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Therapeutically effective amount” is an amount of a Compound of the invention, that when administered to a patient, ameliorates a symptom of the disease. The amount of a Compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

“Preventing” or “prevention” of a disease, disorder, or syndrome includes inhibiting the disease from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome.

“Treating” or “treatment” of a disease, disorder, or syndrome, as used herein, includes (i) inhibiting the disease, disorder, or syndrome, i.e., arresting its development; and (ii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

The compounds disclosed herein also include all pharmaceutically acceptable isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³¹P and ³²P; isotopes of sulfur, such as .sup.³⁵S; isotopes of fluorine, such as ¹⁸F; and isotopes of chlorine, such as ³⁶Cl. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies.

### Embodiments of the Invention

The following paragraphs present a number of embodiments of compounds of the invention. In each instance the embodiment includes both the recited compounds, as well as a single stereoisomer or mixture of stereoisomers thereof, as well as a pharmaceutically acceptable salt thereof.

In one embodiment of the compound of formula I, R³ is H or halo.

In another embodiment, the compound of formula I-1 is a compound of formula Ia, wherein R⁴ is H or F.

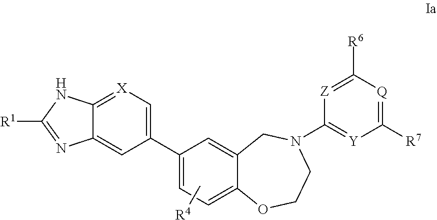

In another embodiment, the compound of formula Ia is a compound of formula Ib.

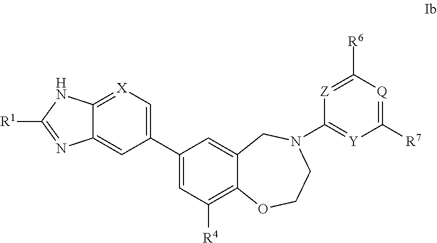

In another embodiment, the compound is a compound of formula Ic-1 or I-c2.

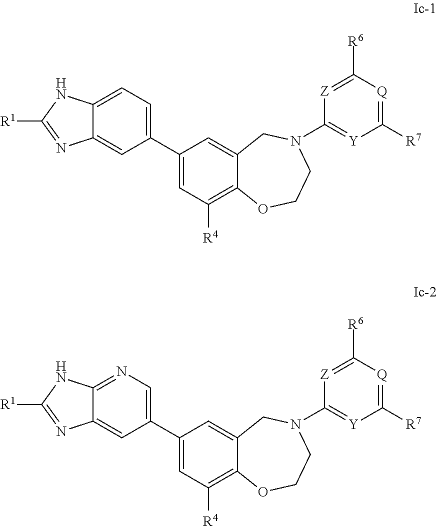

In another embodiment, the compound of formula Ic-2 is in tautomeric formula Ic-3.

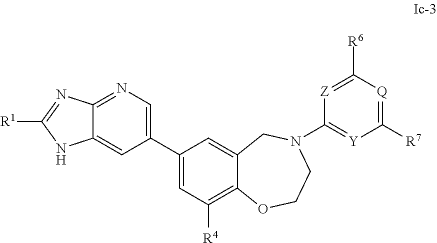

In another embodiment, the compound is a compound of formula Id, wherein R⁴ is H or F.

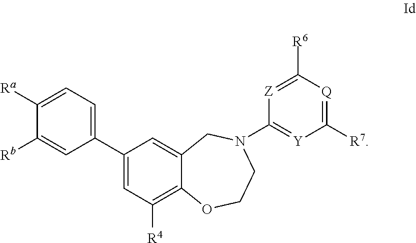

In another embodiment, the compound is a compound of formula Ie.

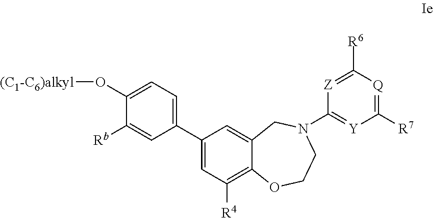

In these and other embodiments of the compound of formula I,

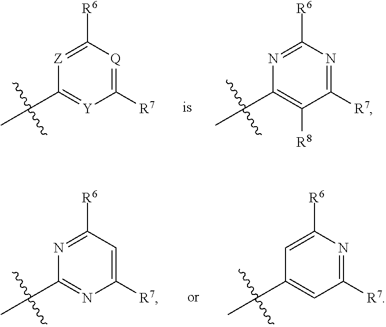

In another embodiment,

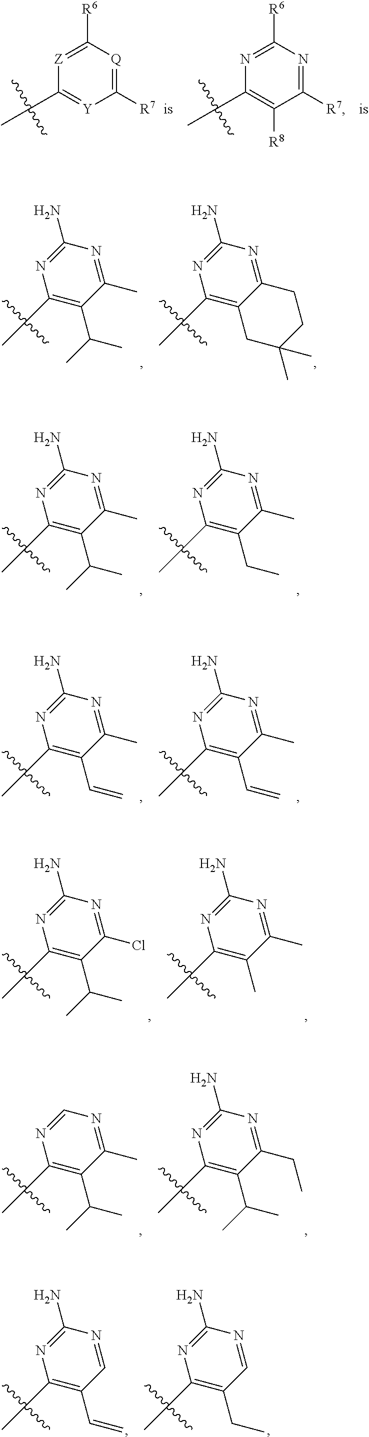

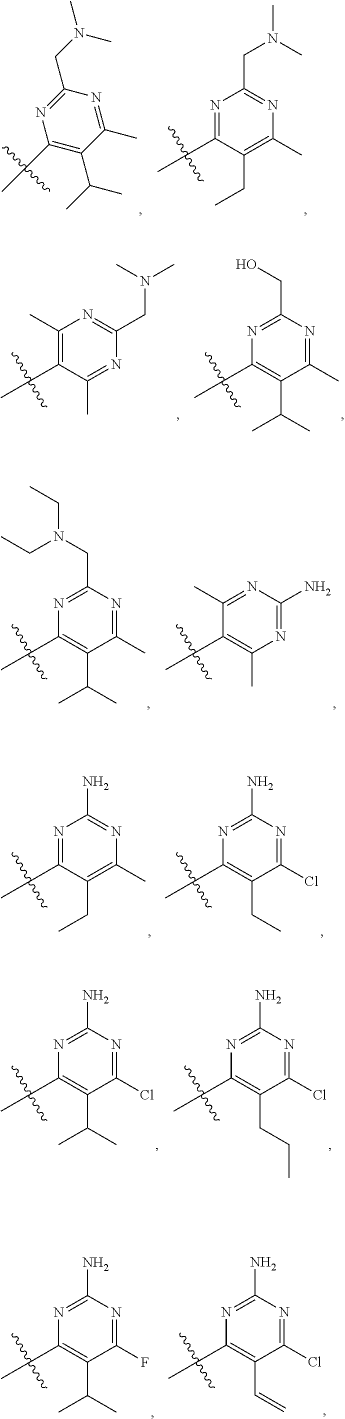

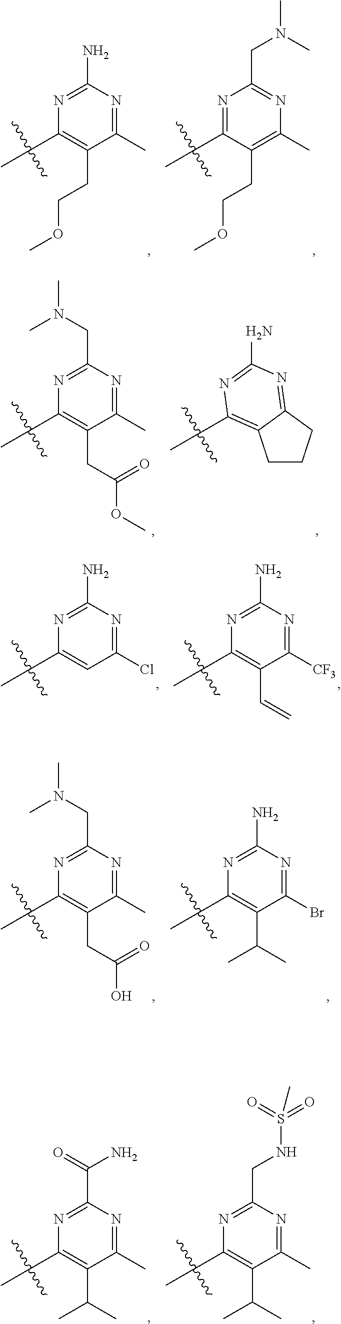

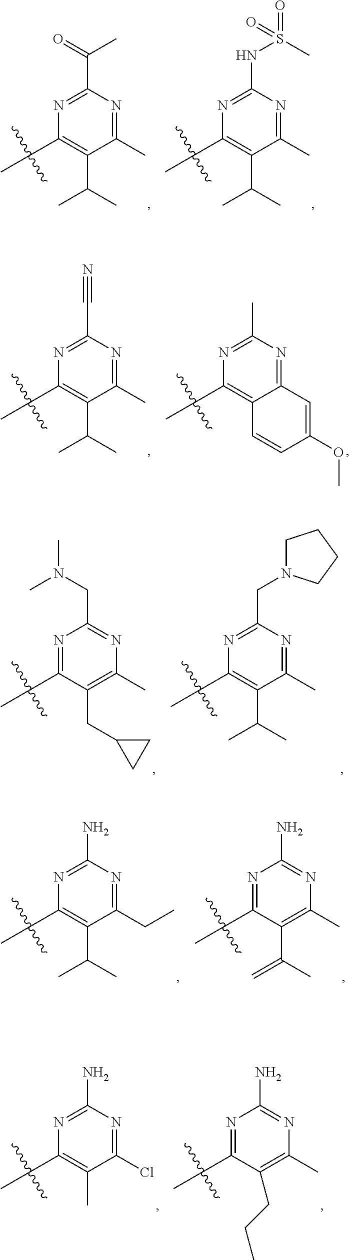

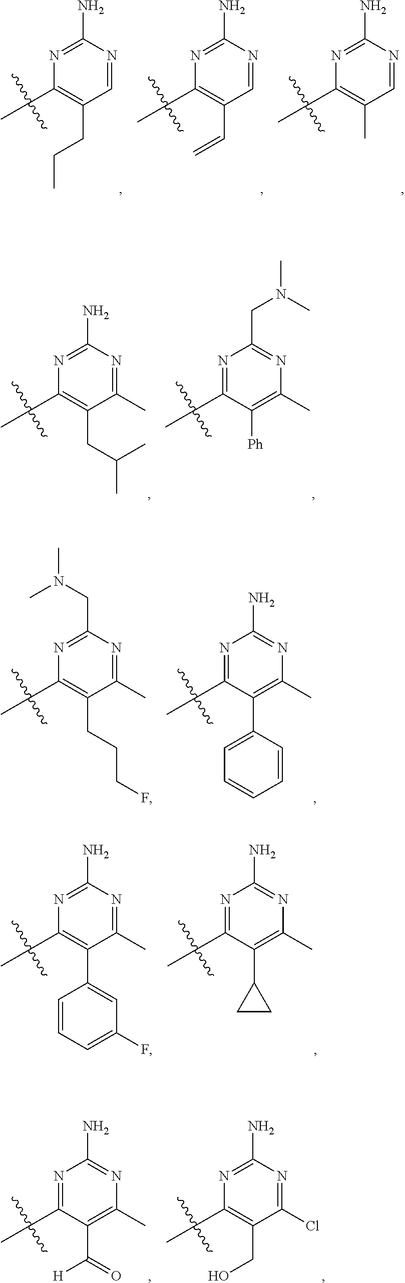

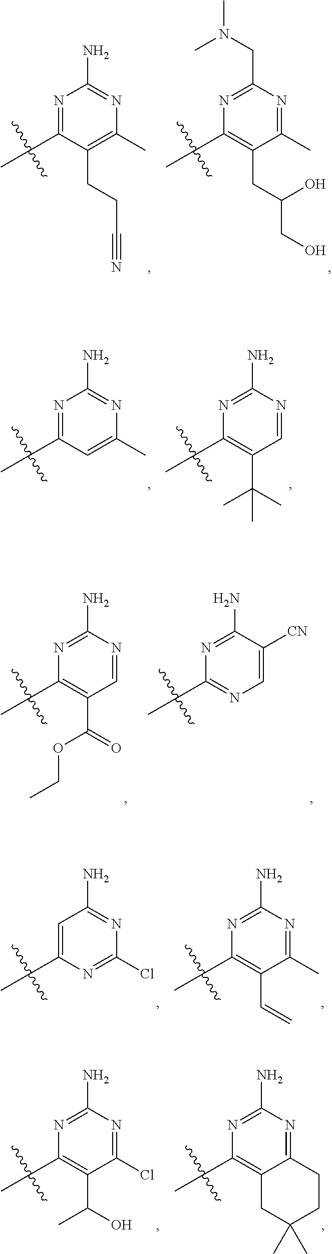

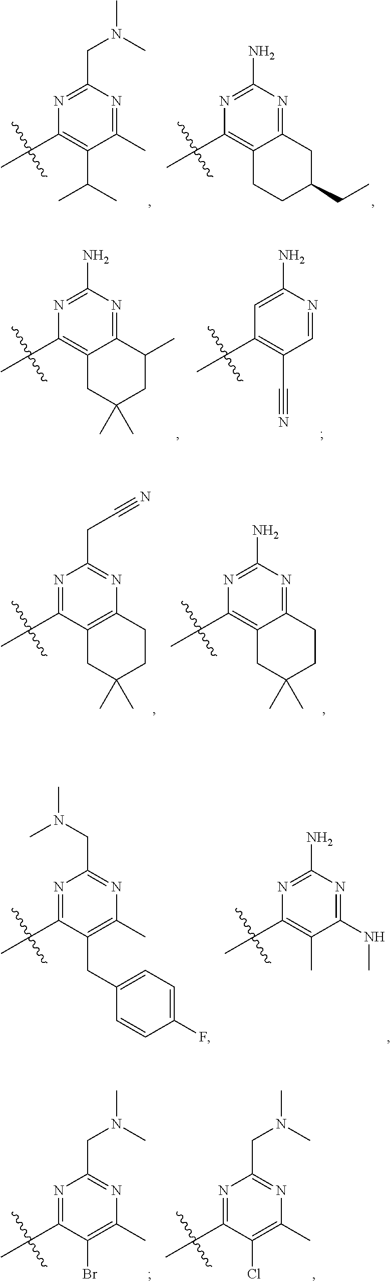

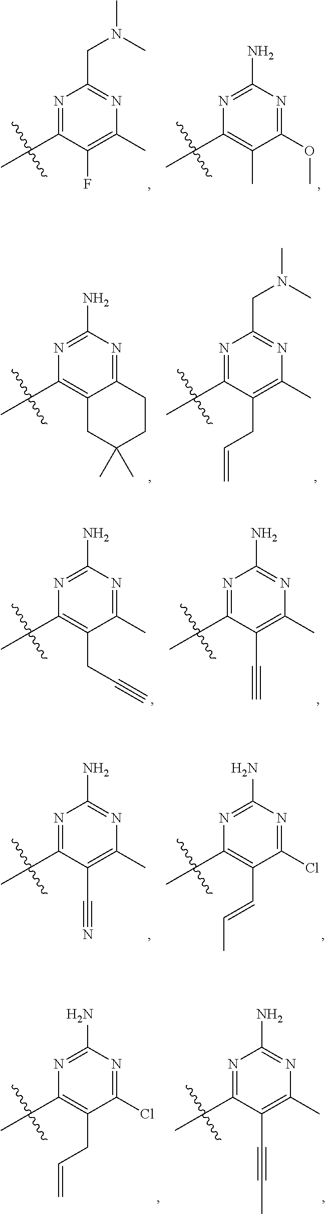

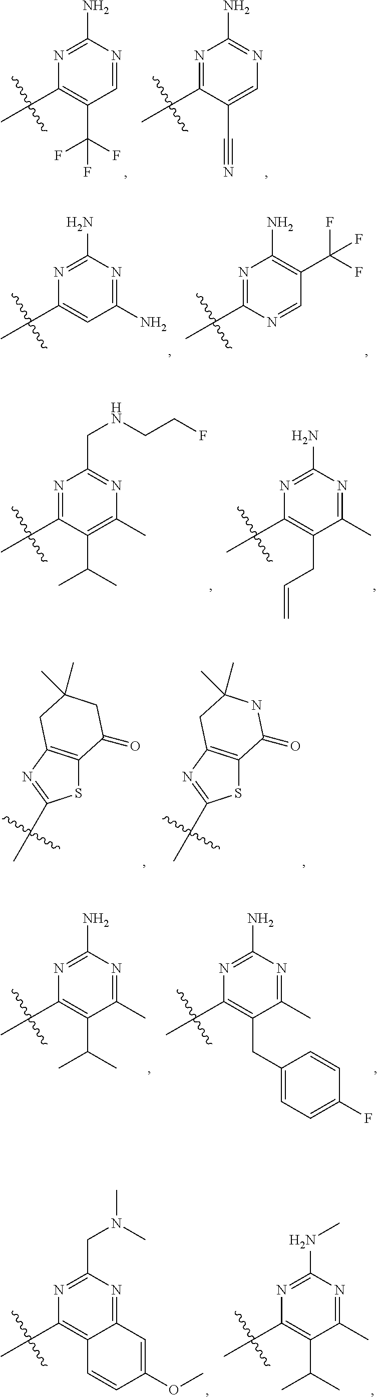

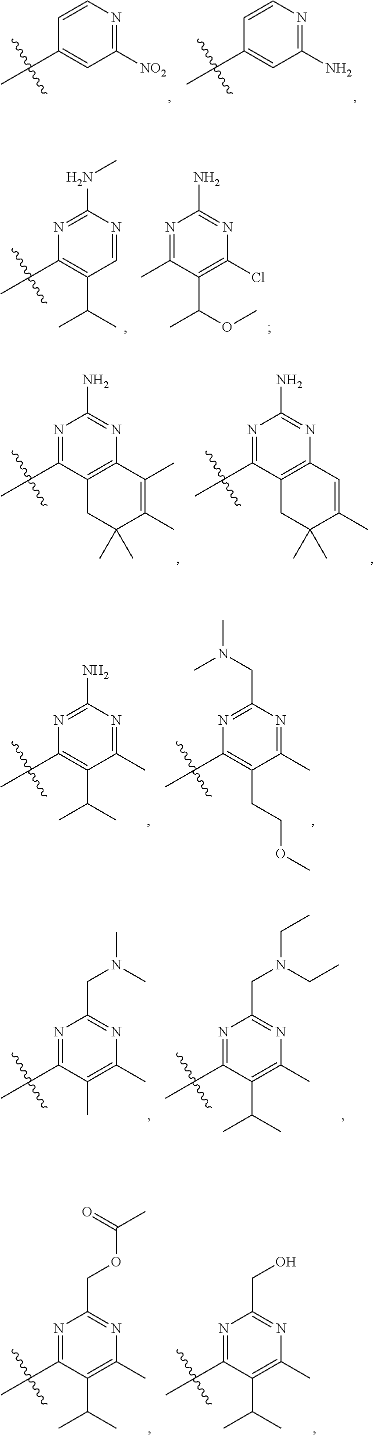

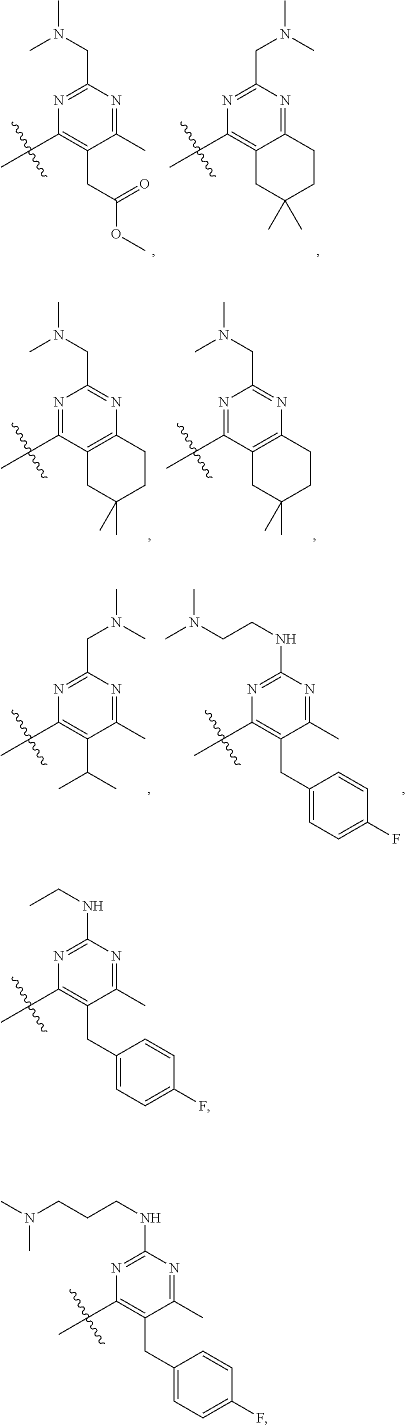

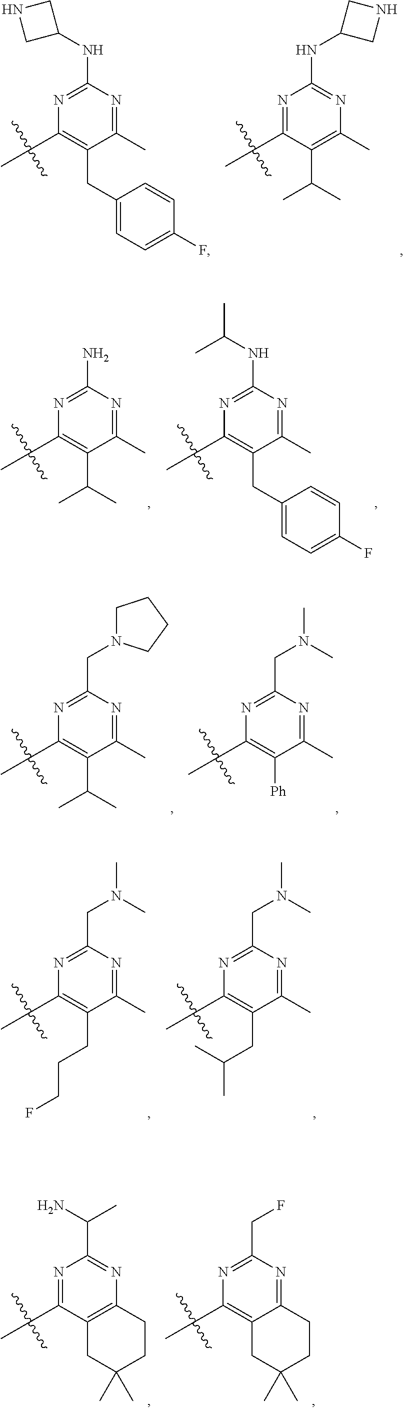

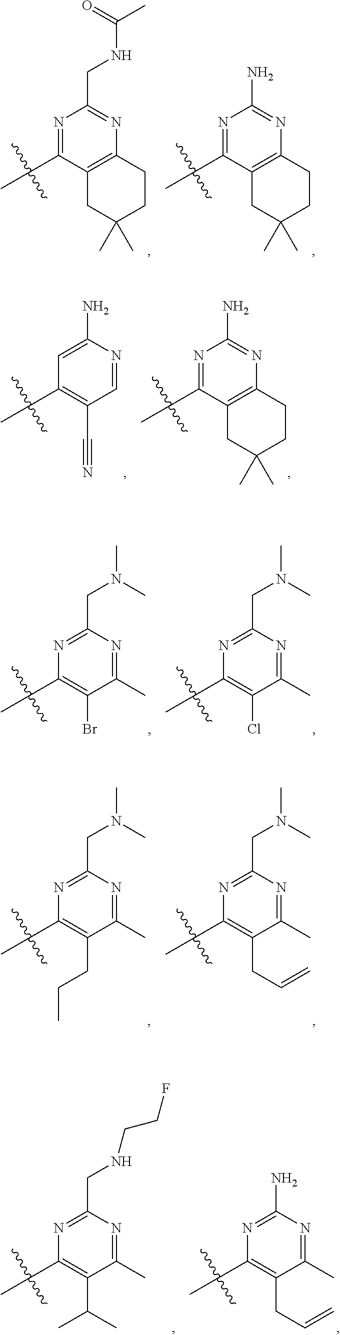

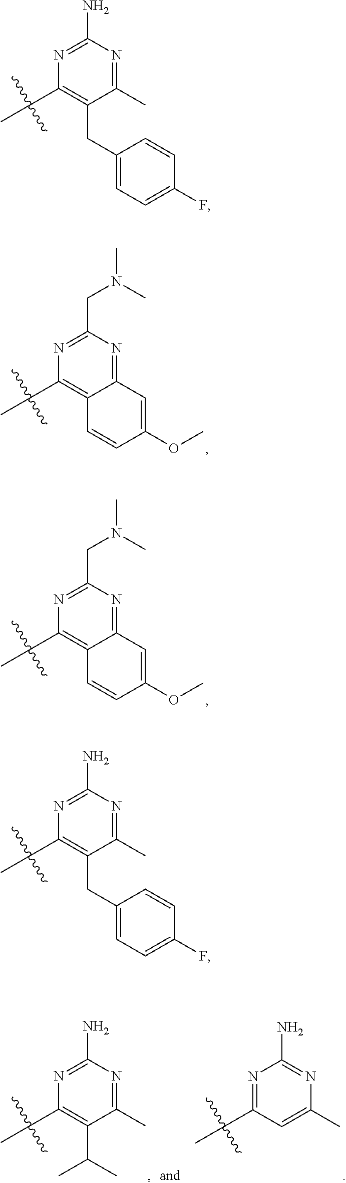

In another embodiment,

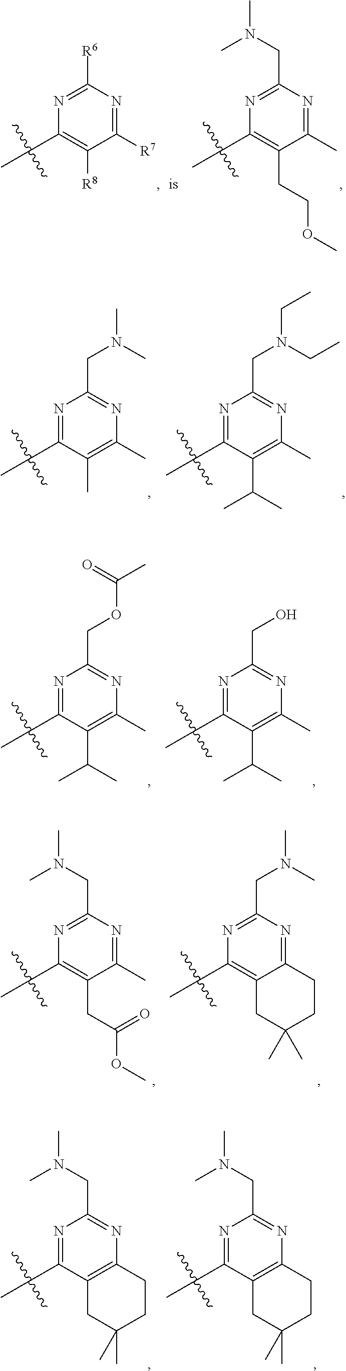

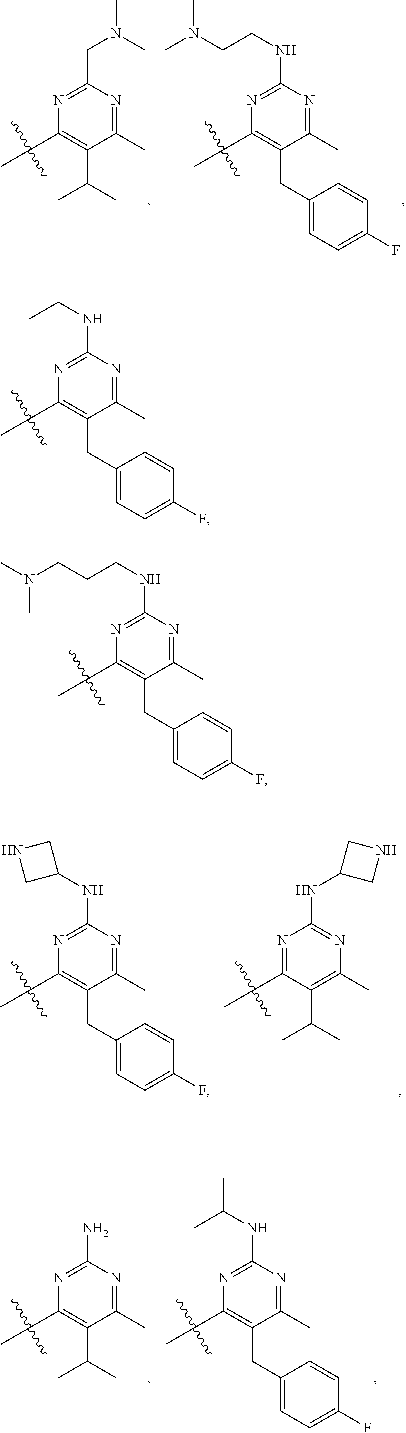

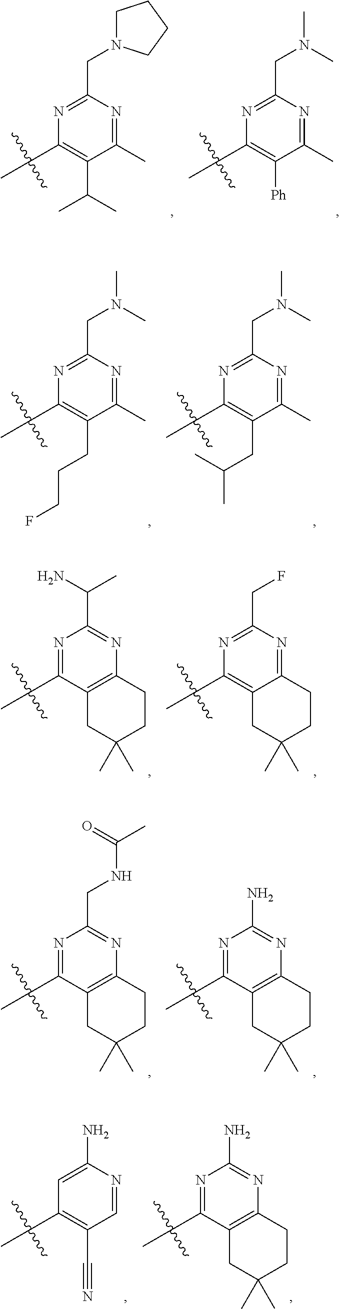

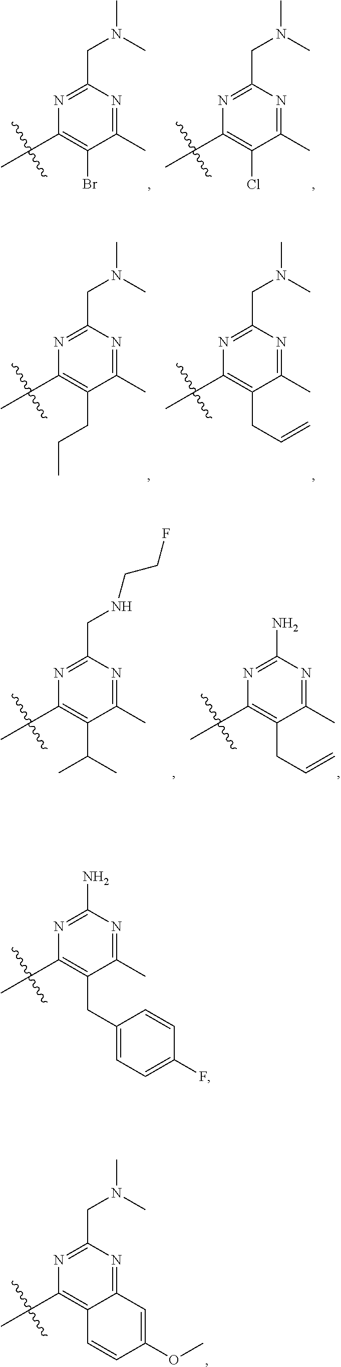

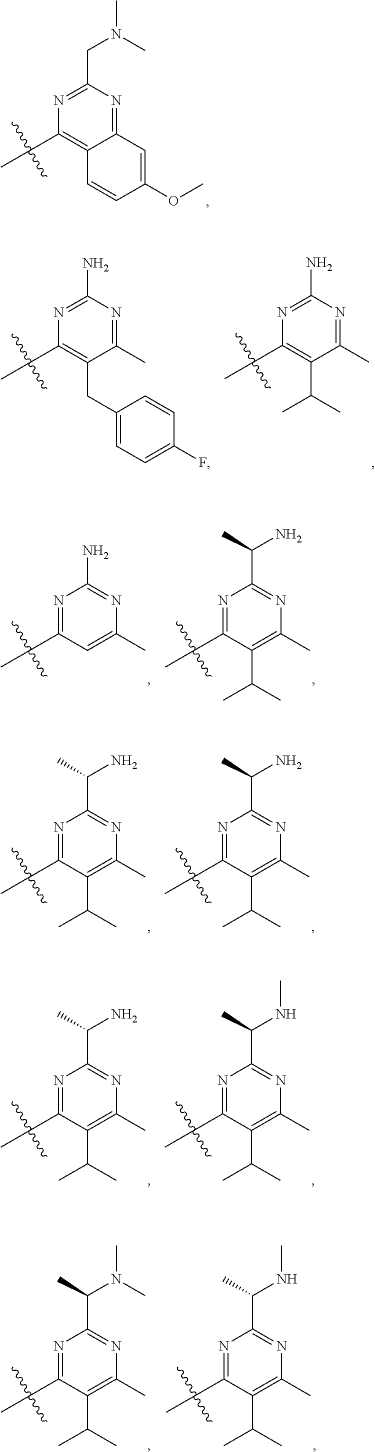

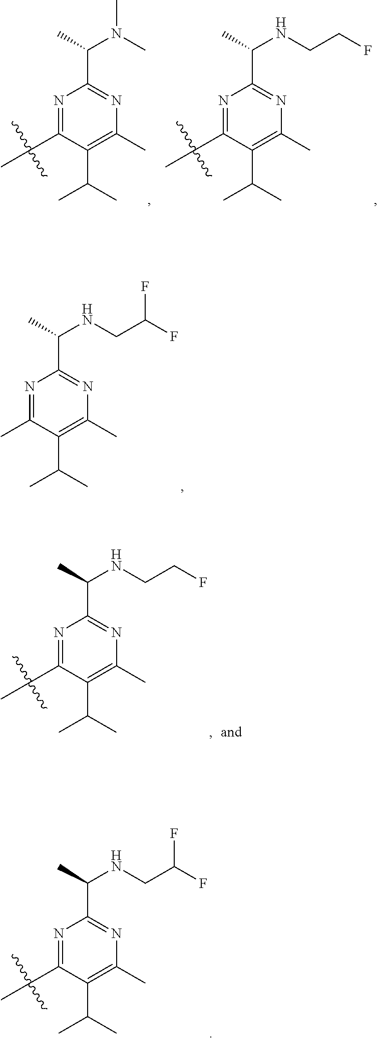 [SMILES: *c1nc([C@H](C)N(C)C)nc(C)c1C(C)C ; *c1nc([C@H](C)NCCF)nc(C)c1C(C)C ; Cc1nc([C@H](C)NCC(F)F)nc(C)c1C(C)C ; *c1nc([C@@H](C)NCCF)nc(C)c1C(C)C ; *c1nc([C@@H](C)NCC(F)F)nc(C)c1C(C)C]

In another embodiment,

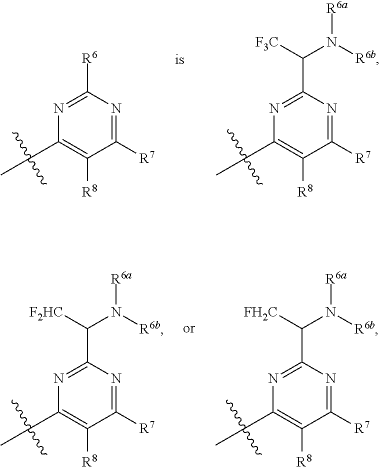

wherein R^{6a} and R^{6b} are each independently H, (C₁-C₆)alkyl, or halo(C₁-C₆)alkyl.

In another embodiment, R¹ in formulae of the compounds described herein is H, (C₁-C₆)alkyl, or NH₂, NHCO(C₁-C₆)alkyl.

In another embodiment, the compound of formulae described herein is:
N,N-dimethyl-1-{4-methyl-6-[7-(2-methyl-1H-benzimidazol-6-yl)-2,3-dihydro-1,4-benzoxazepin-4(5H)-yl]-5-[2-(methyloxy)ethyl]pyrimidin-2-yl}methanamine;
1-{4,5-dimethyl-6-[7-(2-methyl-1H-benzimidazol-6-yl)-2,3-dihydro-1,4-benzoxazepin-4(5H)-yl]pyrimidin-2-yl}-N,N-dimethylmethanamine;
N-ethyl-N-({4-methyl-6-[7-(2-methyl-1H-benzimidazol-5-yl)-2,3-dihydro-1,4-benzoxazepin-4(5H)-yl]-5-(1-methylethyl)pyrimidin-2-yl}methyl)ethanamine;
{4-methyl-6-[7-(2-methyl-1H-benzimidazol-5-yl)-2,3-dihydro-1,4-benzoxazepin-4(5H)-yl]-5-(1-methylethyl)pyrimidin-2-yl}methyl acetate;
{4-methyl-6-[7-(2-methyl-1H-benzimidazol-5-yl)-2,3-dihydro-1,4-benzoxazepin-4(5H)-yl]-5-(1-methylethyl)pyrimidin-2-yl}methanol;
methyl {2-[(dimethylamino)methyl]-4-methyl-6-[7-(2-methyl-1H-benzimidazol-5-yl)-2,3-dihydro-1,4-benzoxazepin-4(5′-1)-yl]pyrimidin-5-yl}acetate;
1-(6,6-dimethyl-4-{7-[4-(methyloxy)-3-{[2-(methyloxy)ethyl]oxy}phenyl]-2,3-dihydro-1,4-benzoxazepin-4(5H)-yl}-5,6,7,8-tetrahydroquinazolin-2-yl)-N,N-dimethylmethanamine;
1-{4-[7-{3-[(difluoromethyl)oxy]-4-(methyloxy)phenyl}-2,3-dihydro-1,4-benzoxazepin-4(5H)-yl]-6,6-dimethyl-5,6,7,8-tetrahydroquinazolin-2-yl}-N,N-dimethylmethanamine; 1-(6,6-dimethyl-4-{7-[4-(methyloxy)-3-(methylsulfonyl)phenyl]-2,3-dihydro-1,4-benzoxazepin-4(5H)-yl}-5,6,7,8-tetrahydroquinazolin-2-yl)-N,N-dimethylmethanamine;
N-[5-(4-{2-[(dimethylamino)methyl]-6-methyl-5-(1-methylethyl)pyrimidin-4-yl}-2,3,4,5-tetrahydro-1,4-benzoxazepin-7-yl)-2-(methyloxy)phenyl]methanesulfonamide;
N′-{5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-yl}-N,N-dimethylethane-1,2-diamine;
N-ethyl-5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-amine;
N′-{5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-yl}-N,N-dimethylpropane-1,3-diamine;
N-azetidin-3-yl-5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-amine;
N-azetidin-3-yl-4-methyl-6-[7-(2-methyl-1H-benzimidazol-5-yl)-2,3-dihydro-1,4-benzoxazepin-4(5H)-yl]-5-(1-methylethyl)pyrimidin-2-amine;
N-[5-{4-[2-amino-6-methyl-5-(1-methylethyl)pyrimidin-4-yl]-2,3,4,5-tetrahydro-1,4-benzoxazepin-7-yl}-2-(methyloxy)phenyl]methanesulfonamide;
methyl (5-{4-[2-amino-6-methyl-5-(1-methylethyl)pyrimidin-4-yl]-2,3,4,5-tetrahydro-1,4-benzoxazepin-7-yl}-1′-1-benzimidazol-2-yl)carbamate;
1-[5-(4-{2-[(dimethylamino)methyl]-6,6-dimethyl-5,6,7,8-tetrahydroquinazolin-4-yl}-2,3,4,5-tetrahydro-1,4-benzoxazepin-7-yl)-2-(methyloxy)phenyl]ethanone;
5-[(4-fluorophenyl)methyl]-4-methyl-6-[7-(2-methyl-1H-benzimidazol-5-yl)-2,3-dihydro-1,4-benzoxazepin-4(5H)-yl]-N-(1-methylethyl)pyrimidin-2-amine;
7-(2-methyl-1H-benzimidazol-5-yl)-4-[6-methyl-5-(1-methylethyl)-2-(pyrrolidin-1-ylmethyl)pyrimidin-4-yl]-2,3,4,5-tetrahydro-1,4-benzoxazepine;
N,N-dimethyl-1-{4-methyl-6-[7-(2-methyl-1H-benzimidazol-5-yl)-2,3-dihydro-1,4-benzoxazepin-4(5H)-yl]-5-phenylpyrimidin-2-yl}methanamine;
1-{5-(3-fluoropropyl)-4-methyl-6-[7-(2-methyl-1H-benzimidazol-5-yl)-2,3-dihydro-1,4-benzoxazepin-4(5H)-yl]pyrimidin-2-yl}-N,N-dimethylmethanamine
N,N-dimethyl-1-{4-methyl-6-[7-(2-methyl-1H-benzimidazol-5-yl)-2,3-dihydro-1,4-benzoxazepin-4(5H)-yl]-5-(2-methylpropyl)pyrimidin-2-yl}methanamine;
1-{6,6-dimethyl-4-[7-(2-methyl-1H-benzimidazol-5-yl)-2,3-dihydro-1,4-benzoxazepin-4(5H)-yl]-5,6,7,8-tetrahydroquinazolin-2-yl}ethanamine;
4-[2-(fluoromethyl)-6,6-dimethyl-5,6,7,8-tetrahydroquinazolin-4-yl]-7-(2-methyl-1H-benzimidazol-5-yl)-2,3,4,5-tetrahydro-1,4-benzoxazepine;
N-({6,6-dimethyl-4-[7-(2-methyl-1H-benzimidazol-5-yl)-2,3-dihydro-1,4-benzoxazepin-4(5H)-yl]-5,6,7,8-tetrahydroquinazolin-2-yl}methyl)acetamide;
4-[7-(1H-benzimidazol-5-yl)-2,3-dihydro-1,4-benzoxazepin-4(5H)-yl]-6,6-dimethyl-5,6,7,8-tetrahydroquinazolin-2-amine;
6-amino-4-[7-(2-methyl-1H-benzimidazol-5-yl)-2,3-dihydro-1,4-benzoxazepin-4(5H)-yl]pyridine-3-carbonitrile;
6,6-dimethyl-4-[7-(2-methyl-1H-benzimidazol-5-yl)-2,3-dihydro-1,4-benzoxazepin-4(5H)-yl]-5,6,7,8-tetrahydroquinazolin-2-amine;
1-{5-bromo-4-methyl-6-[7-(2-methyl-1H-benzimidazol-5-yl)-2,3-dihydro-1,4-benzoxazepin-4(5H)-yl]pyrimidin-2-yl}-N,N-dimethylmethanamine;
1-{5-chloro-4-methyl-6-[7-(2-methyl-1H-benzimidazol-5-yl)-2,3-dihydro-1,4-benzoxazepin-4(5H)-yl]pyrimidin-2-yl}-N,N-dimethylmethanamine;
N,N-dimethyl-1-{4-methyl-6-[7-(2-methyl-1H-benzimidazol-6-yl)-2,3-dihydro-1,4-benzoxazepin-4(5H)-yl]-5-propylpyrimidin-2-yl}methanamine;
N,N-dimethyl-1-{4-methyl-6-[7-(2-methyl-1H-benzimidazol-6-yl)-2,3-dihydro-1,4-benzoxazepin-4(5H)-yl]-5-prop-2-en-1-ylpyrimidin-2-yl}methanamine;
2-fluoro-N-({4-methyl-6-[7-(2-methyl-1H-benzimidazol-5-yl)-2,3-dihydro-1,4-benzoxazepin-4(5H)-yl]-5-(1-methylethyl)pyrimidin-2-yl}methyl)ethanamine;
4-methyl-6-[7-(2-methyl-1H-benzimidazol-5-yl)-2,3-dihydro-1,4-benzoxazepin-4(5H)-yl]-5-prop-2-en-1-ylpyrimidin-2-amine;
4-[7-(1H-benzimidazol-5-yl)-2,3-dihydro-1,4-benzoxazepin-4(5H)-yl]-5-[(4-fluorophenyl)methyl]-6-methylpyrimidin-2-amine;
1-{4-[7-(1H-benzimidazol-5-yl)-2,3-dihydro-1,4-benzoxazepin-4(5H)-yl]-7-(methyloxy)quinazolin-2-yl}-N,N-dimethylmethanamine;
N,N-dimethyl-1-{4-[7-(2-methyl-1H-benzimidazol-5-yl)-2,3-dihydro-1,4-benzoxazepin-4(5H)-yl]-7-(methyloxy)quinazolin-2-yl}methanamine;
5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-amine;
4-[7-(1H-benzimidazol-5-yl)-2,3-dihydro-1,4-benzoxazepin-4(5H)-yl]-6-methyl-5-(1-methylethyl)pyrimidin-2-amine;
4-methyl-6-[7-(2-methyl-1H-benzimidazol-5-yl)-2,3-dihydro-1,4-benzoxazepin-4(5H)-yl]-5-(1-methylethyl)pyrimidin-2-amine;
5-{4-[2-amino-6-methyl-5-(1-methylethyl)pyrimidin-4-yl]-2,3,4,5-tetrahydro-1,4-benzoxazepin-7-yl}-N-ethyl-1H-benzimidazol-2-amine;
4-[7-(1H-benzimidazol-5-yl)-2,3-dihydro-1,4-benzoxazepin-4(5H)-yl]-6-methylpyrimidin-2-amine;
4-[9-fluoro-7-(2-methyl-1H-benzimidazol-6-yl)-2,3-dihydro-1,4-benzoxazepin-4(5H)-yl]-6-methyl-5-(1-methylethyl)pyrimidin-2-amine;
6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-amine;
4-[7-(3H-imidazo[4,5-b]pyridin-6-yl)-2,3-dihydro-1,4-benzoxazepin-4(5H)-yl]-6,6-dimethyl-5,6,7,8-tetrahydroquinazolin-2-amine;
6-{4-[2-amino-6-methyl-5-(1-methylethyl)pyrimidin-4-yl]-2,3,4,5-tetrahydro-1,4-benzoxazepin-7-yl}-3H-imidazo[4,5-b]pyridin-2-amine
5-[(4-fluorophenyl)methyl]-4-[7-(3H-imidazo[4,5-b]pyridin-6-yl)-2,3-dihydro-1,4-benzoxazepin-4(5H)-yl]-6-methylpyrimidin-2-amine
5-[(4-fluorophenyl)methyl]-4-methyl-6-[7-(2-methyl-3H-imidazo[4,5-b]pyridin-6-yl)-2,3-dihydro-1,4-benzoxazepin-4(5H)-yl]pyrimidin-2-amine
1-{4-[7-(3H-imidazo[4,5-b]pyridin-6-yl)-2,3-dihydro-1,4-benzoxazepin-4(5H)-yl]-7-(methyloxy)quinazolin-2-yl}-N,N-dimethylmethanamine
4-methyl-5-(1-methylethyl)-6-[7-(2-methyl-3H-imidazo[4,5-b]pyridin-6-yl)-2,3-dihydro-1,4-benzoxazepin-4(5H)-yl]pyrimidin-2-amine;
4-[7-(3H-imidazo[4,5-b]pyridin-6-yl)-2,3-dihydro-1,4-benzoxazepin-4(5H)-yl]-6-methyl-5-(1-methylethyl)pyrimidine-2-amine;

optionally as a pharmaceutically acceptable salt thereof.

In another aspect, the invention provides a pharmaceutical composition which comprises 1) a compound of formula I, (including I-1, I-2, Ia, Ib, Ic, Id, and Ie here and afterwards unless otherwise specified), as a single stereoisomer or mixture of isomers thereof, selected from Table 1, optionally as a pharmaceutically acceptable salt thereof, and 2) a pharmaceutically acceptable carrier, excipient, and/or diluent thereof.

In another aspect, the invention provides a method of treating disease, disorder, or syndrome where the disease is associated with uncontrolled, abnormal, and/or unwanted cellular activities effected directly or indirectly by PI3K/mTOR which method comprises administering to a human in need thereof a therapeutically effective amount of a compound of formula I, Ia, Ib, Ic, Id, and Ie, optionally as a pharmaceutically acceptable salt or pharmaceutical composition thereof. In another embodiment the disease is cancer.

In an embodiment of this aspect, the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

In another aspect, the invention is directed to a method of treating a disease, disorder, or syndrome which method comprises administering to a patient a therapeutically effective amount of a compound of formula I, optionally as a pharmaceutically acceptable salt thereof, or a pharmaceutical composition comprising a therapeutically effective amount of a compound of formula I, and a pharmaceutically acceptable carrier, excipient, or diluent. In another embodiment the disease is cancer.

In an embodiment of this aspect, the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

In another embodiment, Compounds of the invention which are Compounds of Formula I are also useful as inhibitors of PI3Kα, PI3Kβ and/or mTOR in vivo for studying the in vivo role of PI3Kα and/or mTOR in biological processes, including the diseases described herein. Accordingly, the invention also comprises a method of inhibiting PI3Kα and/or mTOR in vivo comprising administering a compound or composition of the invention to a mammal.

In another embodiment of any of the embodiments as provided above, the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

Another embodiment is directed to a method for identifying a selective inhibitor of a PI3K isozyme, the method comprising: (a) contacting a first cell bearing a first mutation in a PI3K-α with a candidate inhibitor; (b) contacting a second cell bearing a wild type PI3K-α, a PTEN null mutation, or a second mutation in said PI3K-α with the candidate inhibitor; and (c) measuring AKT phosphorylation in said first and said second cells, wherein decreased AKT phosphorylation in said first cell when compared to said second cell identifies said candidate inhibitor as a selective PI3K-α inhibitor. In this and other embodiments provided below, the candidate inhibitor is a Compound of Formula I and of Table I.

Libraries of candidate inhibitor compounds that can be screened using the methods of the present invention may be either prepared or purchased from a number of companies. Synthetic compound libraries are commercially available from, for example, Comgenex (Princeton, N.J.), Brandon Associates (Merrimack, N.H.), Microsource (New Milford, Conn.), and Aldrich (Milwaukee, Wis.). Libraries of candidate inhibitor compounds have also been developed by and are commercially available from large chemical companies. Additionally, natural collections, synthetically produced libraries and compounds are readily modified through conventional chemical, physical, and biochemical means.

Cells to be used in the practice of the screening methods described herein may be primary cells, secondary cells, or immortalized cells (e.g., established cell lines). They may be prepared by techniques well known in the art (for example, cells may be obtained by fine needle biopsy from a patient or a healthy donor) or purchased from immunological and microbiological commercial resources (for example, from the American Type Culture Collection (ATCC), Manassas, Va.). Alternatively or additionally, cells may be genetically engineered to contain, for example, a gene of interest. In a first set of cells, the cells possess a genetic mutation in PI3K-α kinase domain, for example, H1047R. In a second set of cells to be used in the screening assays, the second set of cells possess a genetic mutation in a different kinase catalytic subunit, (for example, a mutation in a helical domain, for example, E545K, or in a different regulatory protein, for example Phosphatase and Tensin Homolog (PTEN). When a candidate inhibitor inhibits phosphorylation, (for example AKT phosphorylation) to a higher degree in the cell possessing the PI3K-α kinase domain genetic mutation when compared to a cell possessing a genetic mutation in a different kinase catalytic subunit, (for example a mutation in a helical domain, for example, E545K, or in a different regulatory protein), then the candidate inhibitor is a selective inhibitor for cancers or tumors that harbor activation mutations in PI3K-α. Conversely, PI3K-α-selective compounds inhibit AKT phosphorylation, PI3K pathway activation, and cell proliferation with greater potency in tumor cells harboring the PI3K-α-H1047R mutation compared to PTEN negative, PI3K-α wild-type, and PI3K-α-E545K backgrounds. Both PTEN inactivation and KRAS activation desensitize cells to the growth inhibitory effects of PI3K-α-selective compounds. A wild-type PI3K-α is illustratively provided in SEQ ID NO: 1 and is encoded by a mRNA of SEQ ID NO: 2.

In some embodiments, the first and second cells used in the screening assay have different genetic backgrounds. In one embodiment, the first cell group has a genetic mutation in a PI3K-α kinase domain. In an illustrative embodiment, the genetic mutation in the first cell group includes a mutation in a mRNA (GenBank Accession No. NM 006218, version NM 006218.2 GI: 54792081 herein disclosed as SEQ ID NO: 2 which encodes a full length PI3K-α having a mutation in the kinase domain. In one embodiment, an exemplary mutation is at a codon (3296, 3297 and 3298), in the kinase domain of SEQ ID NO: 2, wherein the codon is mutated to provide an amino acid other than a histidine at position 1047 of PI3K-α provided in SEQ ID NO: 1. In one exemplary mutation, the histidine at 1047 is mutated to arginine (H1047R). This mutation has been previously reported to be a particularly oncogenic mutation in the PI3K/AKT signaling pathway. The second cell group lacks the mutation of the first test cell group. In one embodiment, an exemplary mutation is at a codon (1790, 1791 and 1792), in the helical domain of SEQ ID NO: 2, wherein the codon is mutated to provide an amino acid other than a glutamic acid at position 542 or 545 of PI3K-α provided in SEQ ID NO: 1. In one exemplary mutation, the glutamic acid at 545 is mutated to lysine (for example, E542K or E545K). This mutation has also been previously reported to be a particularly oncogenic mutation in the PI3K/AKT signaling pathway.

In some embodiments, the second cell group can harbor a mutation in PTEN.

In some embodiments, the first cell group can include various cell lines, including cancer cell lines, for example breast cancer cell lines that may be commercially available from the American Type Culture Collection ((ATCC) American Type Culture Collection, Manassas, Va.) bearing the H1047R het genetic mutation of PI3K-α. In some embodiments, the first cell can include HCT-116, T-47D, MDA-MB-453, SIGOV-3, BT-20 or LS H74T cell lines. In some embodiments, the second cell can include MCF-7, PC3 MCI-H460, SK-BR-3, PC-3, MDA-MB-468, SK-BR-3, MDA-MB-231T, or A549. Each specific cell line can be maintained according to instructions provided upon purchase and are commonly available through the ATCC.

In some embodiments, the first cell group and second cell group can also include non-tumor cell lines that have been transformed with a mutant PI3K-α catalytic subunit, for example. H1047R het or E545K PI3K-α catalytic subunit. Methods of introducing nucleic acids and vectors into isolated cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation (see, e.g., Sambrook et al., supra; Davis et al., Basic Methods in Molecular Biology, 2^{nd} ed., McGraw-Hill Professional, 1995; and Neumann et al., EMBO J., 1: 841 (1982)). There are several methods for eukaryotic cell transformation, either transiently or stably using a variety of expression vectors. Methods for mutating a cell-line, for example NIH 3T3 cells by amplifying a sequence of DNA encoding the mutated PI3K-α catalytic subunit of interest. The amplified PCR mutant PI3K-α construct can be cloned into a viral expression vector, for example, pSX2neo, a Moloney murine leukemia virus (MLV) long terminal repeat-driven expression vector made by inserting a simian virus 40 early promoter-neomycin phosphotransferase gene into pSX2, designed to express high levels of 10A1 MLV Env. Transformation of NIH 3T3 cells can be performed by transfection with a different CaPO₄ coprecipitation technique. After reaching confluence the cells can be transferred into a medium containing 5% FBS without dexamethasone. Morphologically transformed cells can be separated and isolated from mixtures of transformed and nontransformed Env-plasmid-transfected cells by excising the transformed foci from the cell layer with a small-bore pipette (a Pasteur pipette drawn out over a flame to give a fine tip) and aspiration of the foci by the use of a rubber bulb attached to a pipette.

In some embodiments, the methods described herein require that the cells be tested in the presence of a candidate inhibitor, wherein the candidate inhibitor is added to separate exemplary assay wells, each well containing either the first or second cells. The amount of candidate inhibitor can vary, such that a range of inhibitory activities can be determined for the determination of an IC₅₀ for that candidate inhibitor. This can easily be achieved by serially diluting the compound in an appropriate solvent, for example, DMSO and then in the culture medium in which the first and second cells are being incubated in. In some embodiments, the concentration of the candidate inhibitor can range from about 1 pM to about 1 mM concentration. In some embodiments, the candidate inhibitors are added in amounts ranging from about 0.5 nM to about 10 μM. The incubation of candidate inhibitor with first and second cell groups can vary, typically ranging from about 30 minutes to about 60 hours.

In some embodiments, particularly with PI3K-α mediated activity, the cells are stimulated with a growth factor. The selection of growth factor is mediated by the requirements of the cell line, for example, illustrative growth factors can include VEGF, IGF, insulin and heregulin.

In some embodiments, the inhibitory activity of the candidate compounds can be measured using a variety of cellular activities. When cancer cell lines are being used, the inhibition of PI3K mediated activity, e.g. AKT phosphorylation (both at residues S473 and T308), AKT activation, cellular proliferation, and apoptosis resistance in the cells can all be measured. In some embodiments, the amount of AKT phosphorylation in the first and second cell groups can be measured using a phopho-specific antibody (for example AKT 1 (phospho S473, Cat. No. ab8932, AKT1 (phospho T308) Cat. No. ab66134) which are commercially available from AbCam, Cambridge, Mass. Other methods for measuring the inhibition of PI3K-α activity in the first and second cell groups are described in Donahue, A. C. et al., Measuring phosphorylated Akt and other phosphoinositide 3-kinase-regulated phosphoproteins in primary lymphocytes. Methods Enzymol. 2007(434):131-154 which is incorporated herein by reference in its entirety.

In another embodiment, the invention provides a method for determining a treatment regimen for a cancer patient having a tumor comprising a PI3K-α, the method comprising:

determining the presence or absence of a mutation in amino acids 1047 and/or 545 of the PI3K-α;

wherein if the PI3K-α has a mutation at position 1047, the method comprises administering to the cancer patient a therapeutically effective amount of a PI3K-α selective inhibitor compound; or

wherein if the PI3K-α has a mutation at position 545, the method comprises administering to the cancer patient a therapeutically effective amount of a combination of a PI3K-α selective inhibitor and a PI3K-β selective inhibitor, a dual PI3K-α/mTOR selective inhibitor, or a combination of a PI3K-α selective inhibitor and a mTOR selective inhibitor.

In another embodiment, the invention provides a method for determining a treatment regimen for a cancer patient having a tumor comprising a PI3K-α, the method comprising:

determining the presence or absence of a mutation in amino acids 1047 and/or 545 of the PI3K-α;

wherein if the PI3K-α has a mutation at position 1047, the method comprises administering to the cancer patient a therapeutically effective amount of a PI3K-α selective inhibitor compound, a dual PI3K-α/mTOR selective inhibitor, a combination of a PI3K-α selective inhibitor and a mTOR selective inhibitor to the subject; or

wherein if the PI3K-α has a mutation at position 545, the method comprises administering to the cancer patient a therapeutically effective amount of a combination of a PI3K-α selective inhibitor and a PI3K-β selective inhibitor, a dual PI3K-α/mTOR selective inhibitor, or a combination of a PI3K-α selective inhibitor and a mTOR selective inhibitor.

The method of the invention can be used to identify cancer patient populations more likely to benefit from treatment with PI3Kα-selective inhibitors as well as patient populations less likely to benefit.

The invention can be used to further define genetic markers or gene expression signatures which identify PI3Kα inhibitor sensitive tumor subtypes by extended in vitro cell line profiling and in vivo pharmacodynamic and efficacy studies.

In some embodiments, a method for determining a treatment regimen for a cancer patient having the exemplified cancers herein can be readily performed on the basis of the differential activity of PI3K-α selective inhibitors in cancers having a PI3K-α mutated background described herein. In patients in which a tumor cell has been analyzed and assayed to determine whether the tumor harbors a PI3Kα mutation in the kinase domain, for example, a mutation resulting in H1047R, greater efficacy and treatment improvement can be achieved by tailoring a treatment comprising a PI3K-α selective inhibitor. For patients, who have a tumor which does not harbor a mutation in PI3Kαkinase domain, the treatment may require adopting a different treatment regimen, for example, by focusing on delivery of a combination of PI3K-α selective inhibitors and a PI3K-β selective inhibitor, a dual PI3K-α/mTOR selective inhibitor, or a combination of a PI3K-α selective inhibitor and a mTOR selective inhibitor. As indicated above, the PI3K-α selective inhibitors, mTOR selective inhibitors and dual PI3K-α/mTOR selective inhibitors are exemplified in Table 1, and in the detailed description herein.

In some embodiments, methods for determining a treatment regimen comprises determining the presence of a mutation in amino acids 1047 and/or 545 of the PI3K-α in the subject's tumor. This step can be achieved in a variety of ways, using nucleic acid approaches, protein separation approaches or direct immunological approaches using mutation specific antibodies. In some embodiments, presence of a mutation in amino acids 1047 and/or 545 of the PI3K-α in the subject's tumor can be determined using any suitable method for the sequence analysis of amino acids. Examples of suitable techniques include, but are not limited to, western blot analysis, immunoprecipitation, radioimmunoassay (RIA) or enzyme-linked immunoabsorbent assay (ELISA).

In the present invention, reference to position within the amino acid sequence of PI3Kα is made referring to SEQ ID NO: 1. Reference to positions within the nucleotide sequence of the PI3Kα is made referring to SEQ ID NO:2. Specific amino acids in the wild type protein sequence are described using single letter amino acid designation followed by the position in the protein sequence, for example E545 indicates that position 545 is glutamic acid. To represent a substitution at a particular position, the substituted amino acid follows the position, for example E545K indicates that the glutamic acid at position 545 is replaced with a lysine.

Determining the presence or absence of mutations in the sequence of the PI3K-α peptide sequence is generally determined using in vitro methods wherein a tumor sample is used which has been removed from the body of a patient.

Determining the presence or absence of mutations in the amino acid sequence of PI3Kα or a portion thereof, can be done using any suitable method. For example the nucleotide sequence of PI3Kα or a portion thereof maybe determined and the amino acid sequence deduced from the nucleotide sequence or a PI3K-α protein can be interrogated directly.

The nucleotide sequence of the PI3K-α, or a portion thereof, may be determined using any method for the sequence analysis of nucleic acids. Methods for identification of sequence mutation in genes are well known in the art and the mutations in the PI3Kα can be identified by any suitable method. These methods include, but are not limited to, dynamic allele-specific hybridization; the use of molecular beacons; enzyme-based methods, using for example DNA ligase, DNA polymerase or nucleases; PCR based methods, whole genome sequencing; partial genome sequencing; exome sequencing; nucleic acid probe hybridization; and restriction enzyme digestion analysis.

Methods of Direct DNA sequencing are well known in the art, (see for example: Current Protocols in Molecular Biology, edited by Fred M. Ausubel, Roger Brent, Robert E. Kingston, David D. Moore, J. G. Seidman, John A. Smith, Kevin Struhl, and Molecular Cloning: A Laboratory Manual, Joe Sambrook, David W Russel, 3^{rd} edition, Cold Spring Harbor Laboratory Press).) These sequencing protocols include for example, the use of radioactively labeled nucleotides, and nucleotides labeled with a fluorescent dye.

For example, Barbi, S. et al., used the following protocol to sequence the helical domain (exon 9) and the kinase domain (exon 20) of PI3K-α. Normal and tumor DNA was extracted from paraffin-embedded tissue. and amplified using fluorescent dye-labeled primers. the following primer pairs. Primer sequences need to be chosen to uniquely select for a region of DNA, avoiding the possibility of mishybridization to a similar sequence nearby. A commonly used method is BLAST search whereby all the possible regions to which a primer may bind can be seen. Both the nucleotide sequence as well as the primer itself can be BLAST searched. The free NCBI tool Primer-BLAST integrates primer design tool and BLAST search into one application, so does commercial software product such as Beacon Designer, (Premier Biosoft International, Palo Alto Calif.). Mononucleotide repeats should be avoided, as loop formation can occur and contribute to mishybridization. In addition, computer programs are readily available to aid in design of suitable primers. In certain embodiments the nucleic acid probe is labeled for use in a Southern hybridization assay. The nucleic acid probe may be radioactively labeled, fluorescently labeled or is immunologically detectable, in particular is a digoxygenin-labeled (Roche Diagnostics GmbH, Mannheim).

In some embodiments, determining the presence of a helical domain mutation in exon 9 can include the use of forward primer and reverse primers: GGGAAAAATATGACAAAGAAAGC (SEQ ID NO: 3) and CTGAGATCAGCCAAATTCAGTT (SEQ ID NO: 4) respectively and a sequencing primer can include TAGCTAGAGACAATGAATTAAGGGAAA (SEQ ID NO: 5).

For determining a mutation in the kinase domain in exon 20, an exemplary set of primers can include: forward and reverse primers CTCAATGATGCTTGGCTCTG (SEQ ID NO: 6) and TGGAATCCAGAGTGAGCTTTC (SEQ ID NO: 7) respectively and the sequencing primer can include TTGATGACATTGCATACATTCG (SEQ ID NO: 8). The amplification products can then be sequenced. (Barbi, S. et al. J. Experimental and Clinical Cancer Research 2010, 29:32) The sequences are then compared and differences between the wild type PI3K-α sequence and the sequence of the tumor PI3K-α. are determined. The assay could also be performed by only amplifying the tumor DNA and comparing the PI3K-α sequence in the tumor with the sequence of SEQ ID NO: 1.

In some embodiments, the present invention provides polynucleotide sequences comprising polynucleotide sequences in whole or in part from SEQ ID NO: 2 that are capable of hybridizing to the helical region, or the kinase domain of PI3K-α under conditions of high stringency. In some embodiments, the polynucleotides can include sequences complementary to nucleic acid sequences that encode in whole or in part PI3K-α or PI3K-α having specific mutations as described herein. The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

In some embodiments, the present invention provides polynucleotide sequences comprising polynucleotide sequences in whole or in part from SEQ ID NO: 2 that are capable of hybridizing to the helical region, or the kinase domain oPI3K-α under conditions of high stringency. In some embodiments, the present method includes using isolated RNA from a subject's tumor in an assay to determine whether there is a mutation at amino acid at position 1047, 542, or 545 of SEQ ID NO:1, the assay further comprises: (a) reverse transcribing said RNA sample into an equivalent cDNA; (b) amplifying a predetermined region of the cDNA using a pair of nucleic acid probes directed to a predetermined region of the PI3K-α gene; (c) sequencing said amplified cDNA region to obtain a polynucleotide sequence of said amplified cDNA region; and (d) determining whether said amplified cDNA region contains a gene mutation in a codon encoding the amino acid at position 1047, 542, or 545 of SEQ ID NO:1.

In some embodiments, the present methods can employ amplifying a predetermined region of the cDNA by amplifying the cDNA using a pair of nucleic acid primers, a first primer capable of hybridizing stringently to the cDNA upstream of a DNA codon encoding the amino acid at either amino acid 1047 or 542 or 545 of SEQ ID NO:1, and second a nucleic acid primer operable to hybridize stringently to the cDNA downstream of a DNA codon encoding the amino acid at either amino acid 1047 or 542 or 545 of SEQ ID NO:1

In some embodiments, the polynucleotides can include sequences complementary to nucleic acid sequences that encode in whole or in part PI3K-α or PI3K-α having specific mutations as described herein. The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

“High stringency conditions” when used in reference to nucleic acid hybridization comprise conditions equivalent to binding or hybridization at 42 C.° in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/mL denatured salmon sperm DNA followed by washing in a solution comprising 0.1×SSPE, 1.0% SDS at 42 C.° when a probe of about 500 nucleotides in length is employed.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence which is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

In preferred embodiments, hybridization conditions are based on the melting temperature (Tm) of the nucleic acid binding complex and confer a defined “stringency” The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

The term “Tm” refers to the “melting temperature” of a nucleic acid. The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tₘ=81.5+0.41(% G+C), when a nucleic acid is in aqueous solution at 1 M NaCl. The term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences.

In addition, sequence mutations in the PI3Kα can be determined using any sequence-specific nucleic acid detection method allowing detection of single-nucleotide variation, in particular any such method involving complementary base pairing. For example, to determine if the PI3K-α comprises a E545 mutation, the sequence of PI3K-α peptide or a portion thereof comprising nucleotides 1790, 1791 and 1792 of SEQ ID NO:2 (codon corresponding with position 545 in the amino acid sequence), is used in a polymerase chain reaction (PCR) where the oligonucleotide primers allow the amplification of PI3Kα only if the nucleotide at position 1790 is G. If no reaction product is formed then the amino acid at position 545 is mutated. In another example the oligonucleotide primers are designed to allow the amplification of the to allow amplification if the nucleotide at position 3297 is A (codon comprising nucleotides 3296, 3297 and 3298 corresponds with position 1047 of the amino acid sequence). If no reaction product is formed using those primers then the amino acid at position 545 is mutated. Methods for performing PCR are known in the art (see Current Protocols in Molecular Biology, edited by Fred M. Ausubel, Roger Brent, Robert E. Kingston, David D. Moore, J. G. Seidman, John A. Smith, Kevin Struhl. and; Molecular Cloning: A Laboratory Manual, Joe Sambrook, David W Russel, 3^{rd} edition, Cold Spring Harbor Laboratory Press).

Dynamic allele-specific hybridization (DASH) genotyping takes advantage of the differences in the melting temperature in DNA that results from the instability of mismatched base pairs. This technique is well suited to automation. In the first step, a DNA segment is amplified and attached to a bead through a PCR reaction with a biotinylated primer. In the second step, the amplified product is attached to a streptavidin column and washed with NaOH to remove the un-biotinylated strand. An sequence-specific oligonucleotide is then added in the presence of a molecule that fluoresces when bound to double-stranded DNA. The intensity is then measured as temperature is increased until the Tm can be determined. A single nucleotide change will result in a lower than expected Tm (Howell W., Jobs M., Gyllensten U., Brookes A. (1999) Dynamic allele-specific hybridization. A new method for scoring single nucleotide polymorphisms. Nat. Biotechnol. 17(1):87-8). Because DASH genotyping is measuring a quantifiable change in Tm, it is capable of measuring all types of mutations, not just SNPs. Other benefits of DASH include its ability to work with label free probes and its simple design and performance conditions.

Molecular beacons can also be used to detect mutations in a DNA sequences Molecular beacons makes use of a specifically engineered single-stranded oligonucleotide probe. The oligonucleotide is designed such that there are complementary regions at each end and a probe sequence located in between. This design allows the probe to take on a hairpin, or stem-loop, structure in its natural, isolated state. Attached to one end of the probe is a fluorophore and to the other end a fluorescence quencher. Because of the stem-loop structure of the probe, the fluorophore is in close proximity to the quencher, thus preventing the molecule from emitting any fluorescence. The molecule is also engineered such that only the probe sequence is complementary to the to the genomic DNA that will be used in the assay (Abravaya K., Huff J., Marshall R., Merchant B., Mullen C., Schneider G., and Robinson J. (2003) Molecular beacons as diagnostic tools: technology and applications. Clin Chem Lab Med. 41:468-474). If the probe sequence of the molecular beacon encounters its target genomic DNA during the assay, it will anneal and hybridize. Because of the length of the probe sequence, the hairpin segment of the probe will denatured in favor of forming a longer, more stable probe-target hybrid. This conformational change permits the fluorophore and quencher to be free of their tight proximity due to the hairpin association, allowing the molecule to fluoresce. If on the other hand, the probe sequence encounters a target sequence with as little as one non-complementary nucleotide, the molecular beacon will preferentially stay in its natural hairpin state and no fluorescence will be observed, as the fluorophore remains quenched. The unique design of these molecular beacons allows for a simple diagnostic assay to identify SNPs at a given location. If a molecular beacon is designed to match a wild-type allele and another to match a mutant of the allele, the two can be used to identify the genotype of an individual. If only the first probe's fluorophore wavelength is detected during the assay then the individual is homozygous to the wild type. If only the second probe's wavelength is detected then the individual is homozygous to the mutant allele. Finally, if both wavelengths are detected, then both molecular beacons must be hybridizing to their complements and thus the individual must contain both alleles and be heterozygous.

Enzyme-based nucleic acid methods are also suitable and contemplated for determining mutations in the PI3K-α nucleotide sequence. For example, Restriction fragment length polymorphism (RFLP) (discussed in greater detail below) can be used to detect single nucleotide differences. SNP-RFLP makes use of the many different restriction endonucleases and their high affinity to unique and specific restriction sites. By performing a digestion on a genomic sample and determining fragment lengths through a gel assay it is possible to ascertain whether or not the enzymes cut the expected restriction sites. A failure to cut the genomic sample results in an identifiably larger than expected fragment implying that there is a mutation at the point of the restriction site which is rendering it protected from nuclease activity.

The term “functionally equivalent codon” is used herein to refer to codons that encode the same amino acid, such as the six codons for arginine.

In one embodiment of the invention the method comprises at least one nucleic acid probe or oligonucleotide for determining the sequence of the codon that encodes amino acid 1047. In another embodiment the method comprises at least one nucleic acid probe or oligonucleotide for determining the sequence of the codon that encodes amino acid 545. The oligonucleotide is a PCR primer, preferably a set of PCR primers which allows amplification of a PI3Kα nucleic acid sequence fragment only if the codon which encodes amino acid 1047 encodes a histidine. In another method, the PCR primer or set of PCR primers allows the amplification of nucleic acid sequence fragment only if the codon which encodes amino acid 545 encodes a glutamic acid. Determination of suitable PCR primers is routine in the art, (Current Protocols in Molecular Biology, edited by Fred M. Ausubel, Roger Brent, Robert E. Kingston, David D. Moore, J. G. Seidman, John A. Smith, Kevin Struhl; Looseleaf: 0-471-650338-X; CD-ROM: 0-471-30661-4). In addition, computer programs are readily available to aid in design of suitable primers. In certain embodiments the nucleic acid probe is labeled for use in a Southern hybridization assay. The nucleic acid probe may be radioactively labeled, fluorescently labeled or is immunologically detectable, in particular is a digoxygenin-labeled (Roche Diagnostics GmbH, Mannheim).

U.S. Patent Publication 20010016323 discloses methods for detecting point mutations using a fluorescently labeled oligonucleotidemeric probe and fluorescence resonance energy transfer. A point mutation leading to a base mismatch between the probe and the target DNA strand causes the melting temperature of the complex to be lower than the melting temperature for the probe and the target if the probe and target were perfectly matched.

Other suitable methods for detecting single point mutations include those disclosed in, for example, U.S. Patent Publication 2002010665, which involves the use of oligonucleotide probes in array format. Such arrays can include one or more of SEQ ID NOs:3-8. U.S. Patent Publication 20020177157 discloses additional methods for detecting point mutations.

A polynucleotide carrying a point mutation leading to a mutation of PI3K-α kinase domain, for example, H1047R that is the subject of this invention can be identified using one or more of a number of available techniques. However, detection is not limited to the techniques described herein and the methods and compositions of the invention are not limited to these methods, which are provided for exemplary purposes only. Polynucleotide and oligonucleotide probes are also disclosed herein and are within the scope of the invention, and these probes are suitable for one or more of the techniques described below. These include allele-specific oligonucleotide hybridization (ASO), which, in one embodiment, is a diagnostic mutation detection method wherein hybridization with a pair of oligonucleotides corresponding to alleles of a known mutation is used to detect the mutation. Another suitable method is denaturing high performance liquid chromatography (DHPLC), which is a liquid chromatography method designed to identify mutations and polymorphisms based on detection of heteroduplex formation between mismatched nucleotides. Under specified conditions, heteroduplexes elute from the column earlier than homoduplexes because of reduced melting temperature. Analysis can then be performed on individual samples.

An amplified region of the DNA containing the mutation or the wild-type sequence can be analyzed by DHPLC. Use of DHPLC is described in U.S. Pat. Nos. 5,795,976 and 6,453,244, both of which are incorporated herein by reference. A suitable method is that provided by Transgenomic, Inc. (Omaha, Nebr.) using the Transgenomic WAVE® System.

For ASO, a region of genomic DNA or cDNA containing the PI3K-α mutation (H1047R and/or E545K) is amplified by PCR and transferred onto duplicating membranes. This can be performed by dot/slot blotting, spotting by hand, or digestion and Southern blotting. The membranes are prehybridized, then hybridized with a radiolabeled or deoxygenin (DIG) labeled oligonucleotide to either the mutant or wild-type sequences. For the DIG label, detection is performed using chemiluminescent or colorimetric methods. The membranes are then washed with increasing stringency until the ASO is washed from the non-specific sequence. Following autoradiographic exposure, the products are scored for the level of hybridization to each oligonucleotide. Optimally, controls are included for the normal and mutant sequence on each filter to confirm correct stringency, and a negative PCR control is used to check for contamination in the PCR.

The size of the ASO probe is not limited except by technical parameters of the art. Generally, too short a probe will not be unique to the location, and too long a probe may cause loss of sensitivity. The oligonucleotides are preferably 15-21 nucleotides in length, with the mismatch towards the center of the oligonucleotide.

The region of sample DNA on which ASO hybridization is performed to detect the mutation of this invention is preferably amplified by PCR using a forward primer, For exon 9 the forward primer and reverse primers were GGGAAAAATATGACAAAGAAAGC (SEQ ID NO: 3) and CTGAGATCAGCCAAATTCAGTT (SEQ ID NO: 4) respectively and the sequencing primer was TAGCTAGAGACAATGAATTAAGGGAAA (SEQ ID NO: 5), for exon 20 the forward and reverse primers were CTCAATGATGCTTGGCTCTG (SEQ ID NO: 6) and TGGAATCCAGAGTGAGCTTTC (SEQ ID NO: 7) respectively. In this case, amplification by PCR or a comparable method is not necessary but can optionally be performed.

Optionally, one or more than one of the amplified regions described above, (including the 306 nucleotide region generated using primers of SEQ ID NO:3-8, or shorter portions of either of these regions, can be analyzed by sequencing in order to detect the mutation. Sequencing can be performed as is routine in the art. The only limitation on choice of the region to be sequenced, in order to identify the presence of the mutation, is that the region selected for sequencing must include the nucleotide that is the subject of the mutation, The size of the region selected for sequencing is not limited except by technical parameters as is known in the art, and longer regions comprising part or all of the DNA or RNA between selected amplified regions using the primers SEQ ID NOs: 3 & 4 and 6 & 7 disclosed herein can be sequenced.

Variations of the methods disclosed above are also suitable for detecting the mutation. For example, in a variation of ASO, the ASO's are given homopolymer tails with terminal deoxyribonucleotidyl transferase, spotted onto nylon membrane, and covalently bound by UV irradiation. The target DNA is amplified with biotinylated primers and hybridized to the membrane containing the immobilized oligonucleotides, followed by detection. An example of this reverse dot blot technique is the INNO-LIPA kit from Innogenetics (Belgium).

With the identification and sequencing of the mutated gene and the gene product, i.e. SEQ ID NO:1 having a mutation at E545K and H1047R, probes and antibodies raised to the gene product can be used in a variety of hybridization and immunological assays to screen for and detect the presence of either a normal or mutated gene or gene product.

Expression of the mutated gene in heterologous cell systems can be used to demonstrate structure function relationships. Ligating the DNA sequence into a plasmid expression vector to transfect cells is a useful method to test the influence of the mutation on various cellular biochemical parameters. Plasmid expression vectors containing either the entire normal or mutant human or mouse sequence or portions thereof, can be used in vitro mutagenesis experiments which will identify portions of the protein crucial for regulatory function.

The DNA sequence can be manipulated in studies to understand the expression of the gene and its product, and to achieve production of large quantities of the protein for functional analysis, for antibody production, and for patient therapy. Changes in the sequence may or may not alter the expression pattern in terms of relative quantities, tissue-specificity and functional properties.

A number of methods are available for analysis of variant (e.g., mutant or polymorphic) nucleic acid sequences. Assays for detections polymorphisms or mutations fall into several categories, including, but not limited to direct sequencing assays, fragment polymorphism assays, hybridization assays, and computer based data analysis. Protocols and commercially available kits or services for performing multiple variations of these assays are commercially available and known to those of skill in the art. In some embodiments, assays are performed in combination or in combined parts (e.g., different reagents or technologies from several assays are combined to yield one assay). The following illustrative assays may be used to screen and identify nucleic acid molecules containing the mutations of PI3K-α mutation of interest.

### Fragment Length Polymorphism Assays

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a SNP or a mutation will have a different banding pattern than wild type.

### PCR Assays

In some embodiments of the present invention, variant sequences are detected using a PCR-based assay. In some embodiments, the PCR assay comprises the use of oligonucleotide nucleic acid primers that hybridize only to the variant or wild type allele of PI3Kα (e.g., to the region of mutation or multiple mutations). Both sets of primers are used to amplify a sample of DNA. if only the mutant primers result in a PCR product, then the subject's tumor or cancer expresses a somatic mutation in an PI3K-α mutation allele. PCR amplification conditions are tailored to the specific oligonucleotide primers or oligonucleotide probes used, the quality and type of DNA or RNA being screened, and other well known variables that can be controlled using appropriate reagents and/or PCR cycling conditions known to those of ordinary skill in the art.

### RFLP Assays

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from wild-type and mutant controls.

### Direct Sequencing Assays

In some embodiments of the present invention, variant sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, or automated sequencing. The results of the sequencing are displayed using any suitable method. The sequence is examined and the presence or absence of a given SNP or mutation is determined.

### CFLP Assays

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions. The region of interest is first isolated, for example, using PCR. Then, DNA strands are separated by heating. Next, the reactions are cooled to allow intra-strand secondary structure to form. The PCR products are then treated with the CLEAVASE I enzyme to generate a series of fragments that are unique to a given SNP or mutation. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

### Hybridization Assays

In some embodiments of the present invention, variant sequences are detected by hybridization analysis in a hybridization assay. In a hybridization assay, the presence or absence of a given mutation is determined based on the ability of the DNA from the sample to hybridize to a complementary DNA molecule (e.g., a oligonucleotide probe or probes as illustrated herein). A variety of hybridization assays using a variety of technologies for hybridization and detection are available. Relevant and useful hybridization assays for practicing the methods of the present invention are provided below.

### Direct Detection of Hybridization

In some embodiments, hybridization of a probe to the sequence of interest (e.g., a SNP or mutation) is detected directly by visualizing a bound probe (e.g., a Northern or Southern assay; See e.g., Ausabel et al. (eds.) (1991) Current Protocols in Molecular Biology, John Wiley & Sons, NY). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. The unbound probe is removed and the presence of binding is detected by visualizing the labeled probe.

### Detection of Hybridization Using “DNA Chip” Assays

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, an illustrative and commercially available DNA chip assay can include a GENECHIP® (commercially available from Affymetrix, Santa Clara, Calif., USA); See e.g., U.S. Pat. Nos. 6,045,996; 5,925,525; and 5,858,659; each of which is herein incorporated by reference) assay. The GENECHIP® technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

### Enzymatic Detection of Hybridization

In some embodiments of the present invention, hybridization can be detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader. The INVADER assay detects specific mutations in unamplified genomic DNA. The isolated DNA sample is contacted with the first probe specific either for a mutation of the present invention or wild type PI3K-α sequence and allowed to hybridize. Then a secondary probe, specific to the first probe, and containing the fluorescein label, is hybridized and the enzyme is added. Binding is detected by using a fluorescent plate reader and comparing the signal of the test sample to known positive and negative controls.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorometer.

In accordance with the present invention, diagnostic kits are also provided which will include the reagents necessary for the above-described diagnostic screens. For example, kits may be provided which include oligonucleotide probes or PCR primers are present for the detection and/or amplification of mutant PI3K-α, and comparable wild-type PI3K-α-related nucleotide sequences. Again, such probes may be labeled for easier detection of specific hybridization. As appropriate to the various diagnostic embodiments described above, the oligonucleotide probes in such kits may be immobilized to substrates and appropriate controls may be provided. Examples of such oligonucleotide probes include oligonucleotides comprising or consisting of at least one of SEQ ID NOs:3&4 and 6&7.

Determining the presence or absence of mutations in the amino acid sequence of PI3Kα can be determined using any method for the sequence analysis of amino acids. Non-limiting examples include: western blot analysis or ELISA assays, or direct protein sequencing of the PI3Kα in the subject's tumor. In some embodiments, particularly useful antibodies have selectivity for wild type PI3K-α versus the mutant PI3Kα, for example, an antibody useful in the assay would bind to wild type PI3K-α, or a portion wild type PI3Kα, but not to a PI3Kα having a mutation at the amino acid of interest. Particularly useful antibodies could include antibodies which bind the wild type PI3Kα which has histidine at position 1047 but does not bind a mutant PI3Kα which has an amino acid other than histidine, such as arginine, in other words the antibody specifically bind to an epitope comprising histidine at position 1047. Likewise, particularly useful are antibodies which bind the wild type PI3Kα which has glutamic acid at position 545 but does not bind a mutant PI3Kα which has an amino acid other than glutamic acid at position 545, such as lysine at that position.

Another embodiment of the invention provides a method comprising the use of at least one antibody which binds selectively to the wild type PI3Kα protein as compared with binding to a mutated form of PI3Kα. Alternately the antibody binds selectively to a mutated form of PI3Kα as compared with binding to the wild type PI3Kα protein and can differentiate between wild-type PI3Kα and PI3Kα-H1047R or between wild-type PI3Kα and PI3Kα-E545K. Methods for isolating suitable amounts of target protein from a complex mixture in relatively small amounts (less than 1 mg) are commonly known by those skilled in the art. In one illustrative embodiment, a tumor cell or plurality of tumor cells from a subject's tumor or cancer are lysed using commonly available lysing reagents in the presence of protease inhibitors. The lysate is cleared and the supernatant is either electrophoresed and subjected to a Western Blot using mutation specific antibodies, or alternatively, the mutated PI3Kα-H1047R or PI3Kα-E545K are selectively immunoprecipitated and further dissociated from the capture antibody and subjected to Western Blotting or protein sequenced directly.

“Antibody” includes, any immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes and the like.

“Antibody fragment” can refer to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments.

“Chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc) with the desired specificity, affinity, and capability while the constant regions are homologous to the sequences in antibodies derived from another (usually human) to avoid eliciting an immune response in that species.

“Humanized” forms of non-human (e.g., rabbit) antibodies include chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues that are not found in the recipient antibody or in the donor antibody. Most often, the humanized antibody can comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can also comprise at least a portion of an immunoglobulin constant region (Fe), typically that of a human immunoglobulin. Methods used to generate humanized antibodies are well known in the field of immunology and molecular biology.

“Hybrid antibodies” can include immunoglobulin molecules in which pairs of heavy and light chains from antibodies with different antigenic determinant regions are assembled together so that two different epitopes or two different antigens can be recognized and bound by the resulting tetramer.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3-5, and more usually, at least 5 or 8-10 amino acids in a unique spatial conformation.

“Specifically binds” to or shows “specific binding” towards an epitope means that the antibody reacts or associates more frequently, and/or more rapidly, and/or greater duration, and/or with greater affinity with the epitope than with alternative substances.

### Preparation of Antibodies

### Polyclonal Antibodies

Polyclonal antibodies are preferably raised in animals by multiple subcutaneous (sc) or intraperitoneal (ip) injections of the relevant antigen and an adjuvant. Alternatively, antigen may be injected directly into the animal's lymph node (see Kilpatrick et al., Hybridoma, 16:381-389, 1997). An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates or derivatives by combining, e.g., 100 μg of the protein or conjugate (for mice) with 3 volumes of Freund's complete adjuvant and injecting the solution intradermally at multiple sites. One month later, the animals are boosted with 1/5 to 1/10 the original amount of peptide or conjugate in Freund's complete adjuvant by subcutaneous injection at multiple sites. At 7-14 days post-booster injection, the animals are bled and the serum is assayed for antibody titer. Animals are boosted until the titer plateaus. Preferably, the animal is boosted with the conjugate of the same antigen, but conjugated through a different cross-linking reagent. Conjugates also can be made in recombinant cell culture as protein fusions. Also, aggregating agents such as alum are suitably used to enhance the immune response.

### Monoclonal Antibodies

Monoclonal antibodies can be made using the hybridoma method first described by Kohler et al., Nature, 256:495 (1975), or by recombinant DNA methods. In the hybridoma method, a mouse or other appropriate host animal, such as rats, hamster or macaque monkey, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells and are sensitive to a medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Exemplary murine myeloma lines include those derived from MOP-21 and M. C.-11 mouse tumors available from the Salk Institute Cell Distribution Center, San Diego, Calif. USA, and SP-2 or X63-Ag8-653 cells available from the American Type Culture Collection, Rockville, Md. USA. Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can be determined, for example, by BIAcore or Scatchard analysis (Munson et al., Anal. Biochem., 107:220 (1980)).

After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). Suitable culture media for this purpose include, for example, D-MEMO or RPMI 1640 medium. In addition, the hybridoma cells can be grown in vivo as ascites tumors in an animal. The monoclonal antibodies secreted by the subclones are suitably separated from the culture medium, ascites fluid, or serum by conventional immunoglobulin purification procedures such as protein A-Sepharose, hydroxylapatite chromatography, gel electrophoresis, dialysis, or affinity chromatography.

### Recombinant Production of Antibodies

The amino acid sequence of an immunoglobulin of interest can be determined by direct protein sequencing, and suitable encoding nucleotide sequences can be designed according to a universal codon table.

Alternatively, DNA encoding the monoclonal antibodies can be isolated and sequenced from the hybridoma cells using conventional procedures (e.g., by using oligonucleotide probes that are capable of binding specifically to genes encoding the heavy and light chains of the monoclonal antibodies). Sequence determination will generally require isolation of at least a portion of the gene or cDNA of interest. Usually this requires cloning the DNA or mRNA encoding the monoclonal antibodies. Cloning is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library can be constructed by reverse transcription of polyA+mRNA, preferably membrane-associated mRNA, and the library screened using probes specific for human immunoglobulin polypeptide gene sequences. In a preferred embodiment, the polymerase chain reaction (PCR) is used to amplify cDNAs (or portions of full-length cDNAs) encoding an immunoglobulin gene segment of interest (e.g., a light chain variable segment). The amplified sequences can be cloned readily into any suitable vector, e.g., expression vectors, minigene vectors, or phage display vectors. It will be appreciated that the particular method of cloning used is not critical, so long as it is possible to determine the sequence of some portion of the immunoglobulin polypeptide of interest.

One source for RNA used for cloning and sequencing is a hybridoma produced by obtaining a B cell from the transgenic mouse and fusing the B cell to an immortal cell. An advantage of using hybridomas is that they can be easily screened, and a hybridoma that produces a human monoclonal antibody of interest selected. Alternatively, RNA can be isolated from B cells (or whole spleen) of the immunized animal. When sources other than hybridomas are used, it may be desirable to screen for sequences encoding immunoglobulins or immunoglobulin polypeptides with specific binding characteristics. One method for such screening is the use of phage display technology. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference. In one embodiment using phage display technology, cDNA from an immunized transgenic mouse (e.g., total spleen cDNA) is isolated, PCR is used to amplify cDNA sequences that encode a portion of an immunoglobulin polypeptide, e.g., CDR regions, and the amplified sequences are inserted into a phage vector. cDNAs encoding peptides of interest, e.g., variable region peptides with desired binding characteristics, are identified by standard techniques such as panning. The sequence of the amplified or cloned nucleic acid is then determined. Typically the sequence encoding an entire variable region of the immunoglobulin polypeptide is determined, however, sometimes only a portion of a variable region need be sequenced, for example, the CDR-encoding portion. Typically the sequenced portion will be at least 30 bases in length, and more often bases coding for at least about one-third or at least about one-half of the length of the variable region will be sequenced. Sequencing can be carried out on clones isolated from a cDNA library or, when PCR is used, after subcloning the amplified sequence or by direct PCR sequencing of the amplified segment. Sequencing is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of human immunoglobulin genes and cDNAs, an artisan can determine readily, depending on the region sequenced, (i) the germline segment usage of the hybridoma immunoglobulin polypeptide (including the isotype of the heavy chain) and (ii) the sequence of the heavy and light chain variable regions, including sequences resulting from N-region addition and the process of somatic mutation. One source of immunoglobulin gene sequence information is the National Center for Biotechnology Information, National Library of Medicine, National Institutes of Health, Bethesda, Md.

Once isolated, the DNA may be operably linked to expression control sequences or placed into expression vectors, which are then transfected into host cells such as E. coli cells, simian COS cells, Chinese hamster ovary (CHO) cells, or myeloma cells that do not otherwise produce immunoglobulin protein, to direct the synthesis of monoclonal antibodies in the recombinant host cells.

Expression control sequences denote DNA sequences necessary for the expression of an operably linked coding sequence in a particular host organism. The control sequences that are suitable for prokaryotes, for example, include a promoter, optionally an operator sequence, and a ribosome-binding site. Eukaryotic cells are known to utilize promoters, polyadenylation signals, and enhancers.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome-binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking can be accomplished by ligation at convenient restriction sites. If such sites do not exist, synthetic oligonucleotide adaptors or linkers can be used in accordance with conventional practice.

Cell, cell line, and cell culture are often used interchangeably and all such designations include progeny. Transformants and transformed cells include the primary subject cell and cultures derived therefrom without regard for the number of transfers. It also is understood that all progeny may not be precisely identical in DNA content, due to deliberate or inadvertent mutations. Mutant progeny that have the same function or biological activity as screened for in the originally transformed cell are included.

Isolated nucleic acids also are provided that encode specific antibodies, optionally operably linked to control sequences recognized by a host cell, vectors and host cells comprising the nucleic acids, and recombinant techniques for the production of the antibodies, which may comprise culturing the host cell so that the nucleic acid is expressed and, optionally, recovering the antibody from the host cell culture or culture medium.

A variety of vectors are known in the art. Vector components can include one or more of the following: a signal sequence (that, for example, can direct secretion of the antibody), an origin of replication, one or more selective marker genes (that, for example, can confer antibiotic or other drug resistance, complement auxotrophic deficiencies, or supply critical nutrients not available in the media), an enhancer element, a promoter, and a transcription termination sequence, all of which are well known in the art.

Suitable host cells include prokaryote, yeast, or higher eukaryote cells. Suitable prokaryotes include eubacteria, such as Gram-negative or Gram-positive organisms, for example, Enterohacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas, and Streptomyces. In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available, such as Pichia, e.g. P. pastoris, Schizosaccharomyces pombe; Kluyveromyces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

However, interest has been greatest in vertebrate cells, and propagation of vertebrate cells in culture (tissue culture) has become routine. Examples of useful mammalian host cell-lines are Chinese hamster ovary cells, including CHOKI cells (ATCC CCL61) and Chinese hamster ovary cells/−DHFR (DXB-11, DG-44; Urlaub et al, Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse Sertoli cells (TM4, Mather, Biol. Reprod. 23: 243-251 (1980)); monkey kidney cells (CV 1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI38, ATCC CCL 75); human hepatoma cells (Hep G2, HB 8065); mouse mammary tumor (MMT 060562, ATCC CCL51); TRI cells (Mather et al., Annals N.Y. Acad. Sci. 383: 44-68 (1982)); MRC 5 cells and FS4 cells.

The host cells can be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Pat. Re. No. 30,985 can be used as culture media for the host cells. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements also can be included at appropriate concentrations that would be known to those skilled in the art. The culture conditions, such as temperature, pH, and the like, are those previously used with the host cell selected for expression, and will be apparent to the artisan.

The antibody composition can be purified using, for example, hydroxylapatite chromatography, cation or anion exchange chromatography, or preferably affinity chromatography, using the antigen of interest or protein A or protein G as an affinity ligand. Protein A can be used to purify antibodies that are based on human .gamma.1, .gamma.2, or .gamma.4 heavy chains (Lindmark et al., J. Immunol. Meth. 62: 1-13 (1983)). Protein G is recommended for all mouse isotypes and for human .gamma.3 (Guss et al., 20 EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, 25 NJ.) is useful for purification. Other techniques for protein purification such as ethanol precipitation, Reverse Phase HPLC, chromatofocusing, SDS-PAGE, and ammonium sulfate precipitation are also possible depending on the specific binding agent or antibody to be recovered.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3-5, and more usually, at least 5 or 8-10 amino acids in a unique spatial conformation.

“Specifically binds” to or shows “specific binding” towards an epitope means that the antibody reacts or associates more frequently, and/or more rapidly, and/or greater duration, and/or with greater affinity with the epitope than with alternative substances.

In some embodiments, once the subject's tumor has been analyzed to determine whether the tumor harbors a wild type PI3K-α versus a mutant PI3K-α, for example, PI3K-α E545K or PI3K-α H1047R, using any one or more of the assays and methods described above, a treatment regimen can be prepared for the subject. If the subject's tumor harbors a PI3K-α having a mutation at position 1047, (for example, H1047R), the treatment regimen comprises administering to the subject a therapeutically effective amount of a PI3K-α selective inhibitor compound, or a dual PI3K-α/mTOR selective inhibitor, or a combination of a PI3K-α selective inhibitor or a mTOR selective inhibitor. If the subject's tumor harbors a PI3K-α having a mutation at position 545, (for example, E545K), the treatment regimen comprises administering to the subject a therapeutically effective amount of a combination of a PI3K-α selective inhibitor and a PI3K-β selective inhibitor, a dual PI3K-α/mTOR selective inhibitor, or a combination of a PI3K-α selective inhibitor and a mTOR selective inhibitor.

In another embodiment, the present invention provides kits comprising materials useful for carrying out the methods of the invention. The diagnostic/screening procedures described herein may be performed by diagnostic laboratories, experimental laboratories, or practitioners. The invention provides kits which can be used in these different settings.

Basic materials and reagents required for identifying a PI3K-α mutation in a subject's tumor or cancer according to methods of the present invention may be assembled together in a kit. In certain embodiments, the kit comprises at least one PI3K-α amino acid sequence determining reagent that specifically detects a mutation in a nucleic acid or protein obtained from a subject's tumor disclosed herein, and instructions for using the kit according to one or more methods of the invention. Each kit necessarily comprises reagents which render the procedure specific. Thus, for detecting mRNA harboring the PI3K-α H1047R or E545K mutation, the reagent will comprise a nucleic acid probe complementary to mRNA, such as, for example, a cDNA or an oligonucleotide. The nucleic acid probe may or may not be immobilized on a substrate surface (e.g., a microarray). For detecting a polypeptide product encoded by at least one PI3K-α mutation gene, the reagent will comprise an antibody that specifically binds to the mutated PI3K-α or a wild-type PI3K-α.

Depending on the procedure, the kit may further comprise one or more of extraction buffer and/or reagents, amplification buffer and/or reagents, hybridization buffer and/or reagents, immunodetection buffer and/or reagents, labeling buffer and/or reagents, and detection means. Protocols for using these buffers and reagents for performing different steps of the procedure may also be included in the kit.

Reagents may be supplied in a solid (e.g., lyophilized) or liquid form. Kits of the present invention may optionally comprise one or more receptacles for mixing samples and/or reagents (e.g., vial, ampoule, test tube, ELISA plate, culture plate, flask or bottle) for each individual buffer and/or reagent. Each component will generally be suitable as aliquoted in its respective container or provided in a concentrated form. Other containers suitable for conducting certain steps for the disclosed methods may also be provided. The individual containers of the kit are preferably maintained in close confinement for commercial sale.

In certain embodiments, the kits of the present invention further comprise control samples. For example, a kit may include samples of total mRNA derived from tissue of various physiological states, such as, for example, wild-type PI3K-α, PI3K-α H1047R mRNA or PI3K-α E545K mRNA to be used as controls. In other embodiments, the inventive kits comprise at least one prostate disease expression profile map as described herein for use as comparison template. Preferably, the expression profile map is digital information stored in a computer-readable medium.

Instructions for using the kit according to one or more methods of the invention may comprise instructions for processing the prostate tissue sample and/or performing the test, instructions for interpreting the results as well as a notice in the form prescribed by a governmental agency (e.g., FDA) regulating the manufacture, use or sale of pharmaceuticals or biological products.

### Representative Compounds

The structures and names of the compounds of the invention are depicted Table 1. Each entry in the Table is meant to include the compound as shown, as well as single stereoisomer or mixture of stereoisomers thereof, and pharmaceutically acceptable salts thereof. Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS). Specifically, names in Table 1 were generated using ACD/Labs naming software 8.00 release, product version 8.08 or later.

TABLE 1

| CMPD | Structure | Name |
|---|---|---|
| 1 | 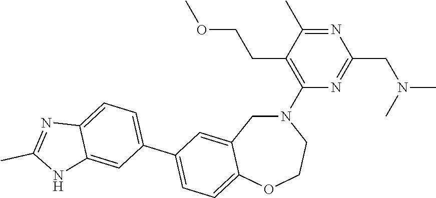 | N,N-dimethyl-1-{4-methyl-6- [7-(2-methyl-1H- benzimidazol-6-yl)-2,3- dihydro-1,4-benzoxazepin- 4(5H)-yl]-5-[2- (methyloxy)ethyl]pyrimidin-2- yl}methanamine |
| 2 | 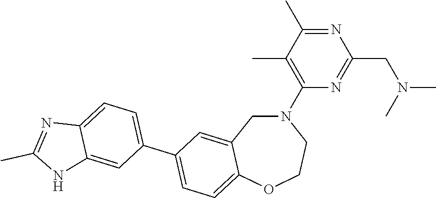 | I-{4,5-dimethyl-6-[7-(2- methyl-1H-benzimidazol-6-yl)- 2,3-dihydro-1,4-benzoxazepin- 4(5H)-yl]pyrimidin-2-yl}-N,N- dimethylmethanamine |
| 3 | 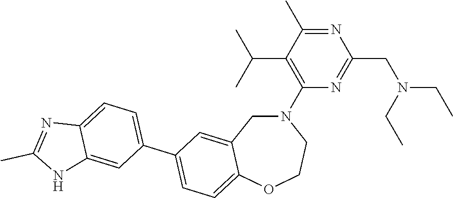 [SMILES: CCN(CC)Cc1nc(C)c(C(C)C)c(N2CCOc3ccc(-c4ccc5nc(C)[nH]c5c4)cc3C2)n1] | N-ethyl-N-({4-methyl-6-[7-(2- methyl-1H-benzimidazol-5-yl)- 2,3-dihydro-1,4-benzoxazepin- 4(5H)-yl]-5-(1- methylethyl)pyrimidin-2- yl}methyl)ethanamine |
| 4 | 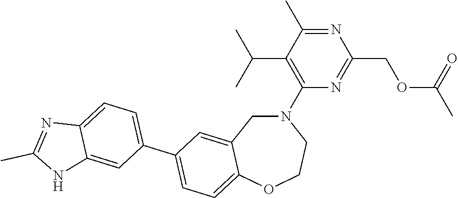 [SMILES: CC(=O)OCc1nc(C)c(C(C)C)c(N2CCOc3ccc(-c4ccc5nc(C)[nH]c5c4)cc3C2)n1] | {4-methyl-6-[7-(2-methyl-1H- benzimidazol-5-yl)-2,3- dihydro-1,4-benzoxazepin- 4(5H)-yl]-5-(1- methylethyl)pyrimidin-2- yl}methyl acetate |
| 5 | 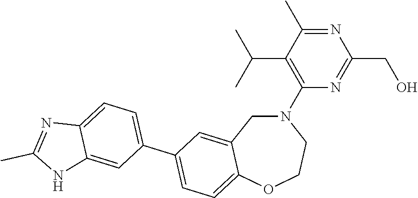 [SMILES: Cc1nc2ccc(-c3ccc4c(c3)CN(c3nc(CO)nc(C)c3C(C)C)CCO4)cc2[nH]1] | {4-methyl-6-[7-(2-methyl-1H- benzimidazol-5-yl)-2,3- dihydro-1,4-benzoxazepin- 4(5H)-yl]-5-(1- methylethyl)pyrimidin-2- yl}methanol |
| 6 | 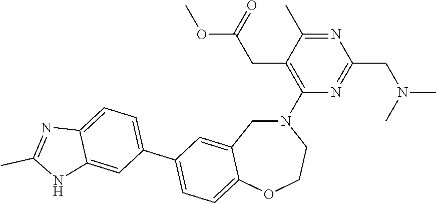 | methyl {2- [(dimethylamino)methyl]-4- methyl-6-[7-(2-methyl-1H- benzimidazol-5-yl)-2,3- dihydro-1,4-benzoxazepin- 4(5H)-yl]pyrimidin-5- yl}acetate |
| 7 | 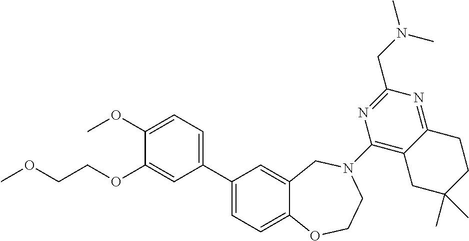 | 1-(6,6-dimethyl-4-{7-[4- (methyloxy)-3-{[2- (methyloxy)ethyl]oxy}phenyl]- 2,3-dihydro-1,4-benzoxazepin- 4(5H)-yl}-5,6,7,8- tetrahydroquinazolin-2-yl)- N,N-dimethylmethanamine |
| 8 | 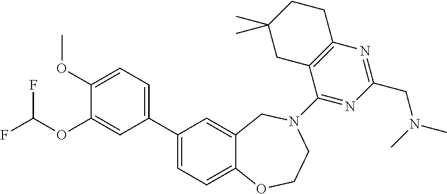 [SMILES: COc1ccc(-c2ccc3c(c2)CN(c2nc(CN(C)C)nc4c2CC(C)(C)CC4)CCO3)cc1OC(F)F] | 1-{4-[7-{3- [(difluoromethyl)oxy]-4- (methyloxy)phenyl}-2,3- dihydro-1,4-benzoxazepin- 4(5H)-yl]-6,6-dimethyl- 5,6,7,8-tetrahydroquinazolin-2- yl}-N,N-dimethylmethanamine |
| 9 | 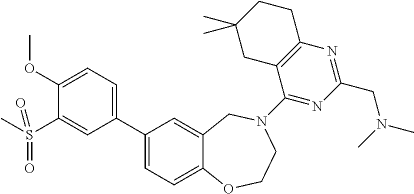 | 1-(6,6-dimethyl-4-{7-[4- (methyloxy)-3- (methylsulfonyl)phenyl]-2,3- dihydro-1,4-benzoxazepin- 4(5H)-yl}-5,6,7,8- tetrahydroquinazolin-2-yl)- N,N-dimethylmethanamine |
| 10 | 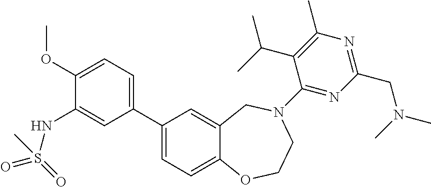 [SMILES: COc1ccc(-c2ccc3c(c2)CN(c2nc(CN(C)C)nc(C)c2C(C)C)CCO3)cc1NS(C)(=O)=O] | N-[5-(4-{2- (dimethylamino)methyl]-6- methyl-5-(1- methylethyl)pyrimidin-4-yl}- 2,3,4,5-tetrahydro-1,4- benzoxazepin-7-yl)-2- (methyloxy)phenyl] methanesulfonamide |
| 11 | 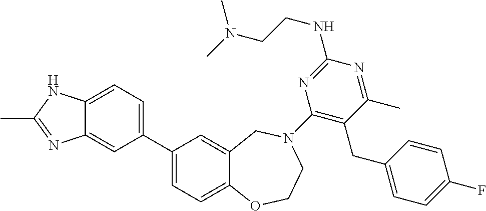 [SMILES: Cc1nc2cc(-c3ccc4c(c3)CN(c3nc(NCCN(C)C)nc(C)c3Cc3ccc(F)cc3)CCO4)ccc2[nH]1] | N′-{5-[(4- fluorophenyl)methyl]-4- methyl-6-[7-(2-methyl-1H- benzimidazol-5-yl)-2,3- dihydro-1,4-benzoxazepin- 4(5H)-yl]pyrimidin-2-yl}-N,N- dimethylethane-1,2-diamine |
| 12 | 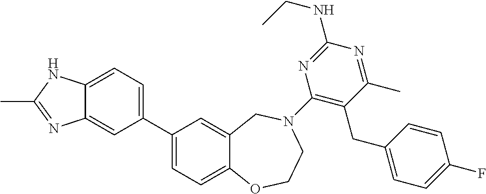 [SMILES: CCNc1nc(C)c(Cc2ccc(F)cc2)c(N2CCOc3ccc(-c4ccc5[nH]c(C)nc5c4)cc3C2)n1] | N-ethyl-5-[(4- fluorophenyl)methyl]-4- methyl-6-[7-(2-methyl-1H- benzimidazol-5-yl)-2,3- dihydro-1,4-benzoxazepin- 4(5H)-yl]pyrimidin-2-amine |
| 13 | 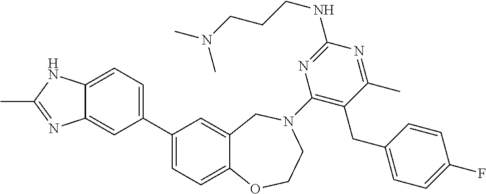 [SMILES: Cc1nc2cc(-c3ccc4c(c3)CN(c3nc(NCCCN(C)C)nc(C)c3Cc3ccc(F)cc3)CCO4)ccc2[nH]1] | N′-{5-[(4- fluorophenyl)methyl]-4- methyl-6-[7-(2-methyl-1H- benzimidazol-5-yl)-2,3- dihydro-1,4-benzoxazepin- 4(5H)-yl]pyrimidin-2-yl}-N,N- dimethylpropane-1,3-diamine |
| 14 | 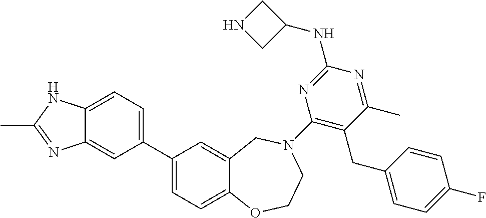 [SMILES: Cc1nc2cc(-c3ccc4c(c3)CN(c3nc(NC5CNC5)nc(C)c3Cc3ccc(F)cc3)CCO4)ccc2[nH]1] | N-azetidin-3-yl-5-[(4- fluorophenyl)methyl]-4- methyl-6-[7-(2-methyl-1H- benzimidazol-5-yl)-2,3- dihydro-1,4-benzoxazepin- 4(5H)-yl]pyrimidin-2-amine |
| 15 | 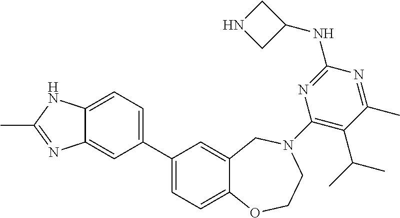 | N-azetidin-3-yl-4-methyl-6-[7- (2-methyl-1H-benzimidazol-5- yl)-2,3-dihydro-1,4- benzoxazepin-4(5H)-yl]-5-(1- methylethyl)pyrimidin-2-amine |
| 16 | 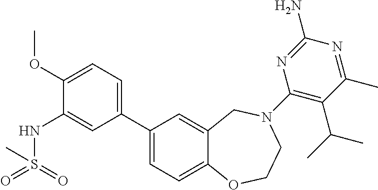 [SMILES: COc1ccc(-c2ccc3c(c2)CN(c2nc(N)nc(C)c2C(C)C)CCO3)cc1NS(C)(=O)=O] | N-[5-{4-[2-amino-6-methyl-5- (1-methylethyl)pyrimidin-4- yl]-2,3,4,5-tetrahydro-1,4- benzoxazepin-7-yl}-2- (methyloxy)phenyl] methanesulfonamide |
| 17 | 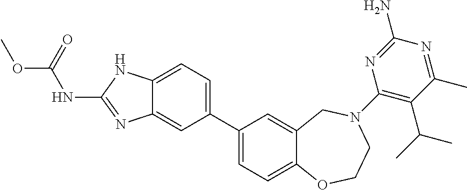 | methyl (5-{4-[2-amino-6- methyl-5-(1- methylethyl)pyrimidin-4-yl]- 2,3,4,5-tetrahydro-1,4- benzoxazepin-7-yl}-1H- benzimidazol-2-yl)carbamate |
| 18 | 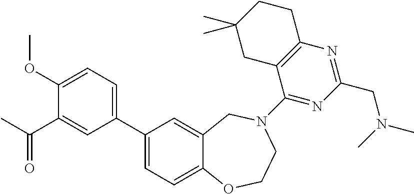 [SMILES: COc1ccc(-c2ccc3c(c2)CN(c2nc(CN(C)C)nc4c2CC(C)(C)CC4)CCO3)cc1C(C)=O] | 1-[5-(4-{2- [(dimethylamino)methyl]-6,6- dimethyl-5,6,7,8- tetrahydroquinazolin-4-yl}- 2,3,4,5-tetrahydro-1,4- benzoxazepin-7-yl)-2- (methyloxy)phenyl]ethanone |
| 19 | 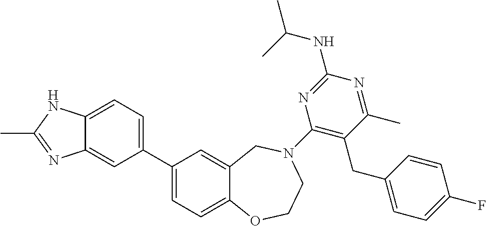 [SMILES: Cc1nc2cc(-c3ccc4c(c3)CN(c3nc(NC(C)C)nc(C)c3Cc3ccc(F)cc3)CCO4)ccc2[nH]1] | 5-[(4-fluorophenyl)methyl]-4- methyl-6-[7-(2-methyl-1H- benzimidazol-5-yl)-2,3- dihydro-1,4-benzoxazepin- 4(5H)-yl]-N-(1- methylethyl)pyrimidin-2-amine |
| 20 | 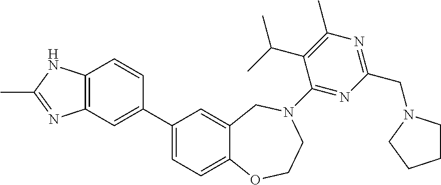 [SMILES: Cc1nc2cc(-c3ccc4c(c3)CN(c3nc(CN5CCCC5)nc(C)c3C(C)C)CCO4)ccc2[nH]1] | 7-(2-methyl-1H-benzimidazol- 5-yl)-4-[6-methyl-5-(1- methylethyl)-2-(pyrrolidin-1- ylmethyl)pyrimidin-4-yl]- 2,3,4,5-tetrahydro-1,4- benzoxazepine |
| 21 | 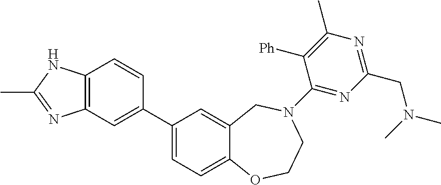 | N,N-dimethyl-1-{4-methyl-6- [7-(2-methyl-1H- benzimidazol-5-yl)-2,3- dihydro-1,4-benzoxazepin- 4(5H)-yl]-5-phenylpyrimidin- 2-yl}methanamine |
| 22 | 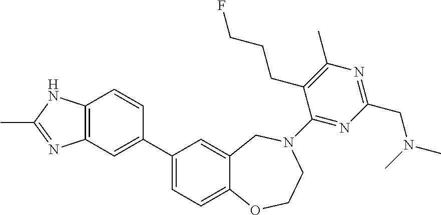 | 1-{5-(3-fluoropropyl)-4- methyl-6-[7-(2-methyl-1H- benzimidazol-5-yl)-2,3- dihydro-1,4-benzoxazepin- 4(5H)-yl]pyrimidin-2-yl}-N,N- dimethylmethanamine |
| 23 | 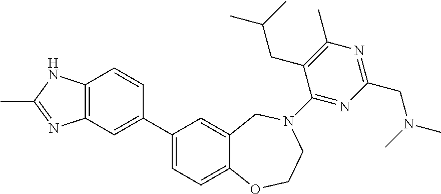 | N,N-dimethyl-1-{4-methyl-6- [7-(2-methyl-1H- benzimidazol-5-yl)-2,3- dihydro-1,4-benzoxazepin- 4(5H)-yl]-5-(2- methylpropyl)pyrimidin-2- yl}methanamine |
| 24 | 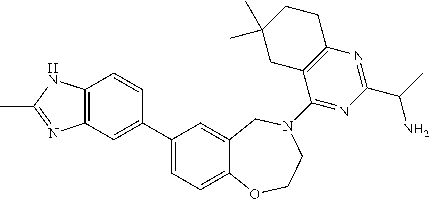 [SMILES: Cc1nc2cc(-c3ccc4c(c3)CN(c3nc(C(C)N)nc5c3CC(C)(C)CC5)CCO4)ccc2[nH]1] | 1-{6,6-dimethyl-4-[7-(2- methyl-1H-benzimidazol-5-yl)- 2,3-dihydro-1,4-benzoxazepin- 4(5H)-yl]-5,6,7,8- tetrahydroquinazolin-2- yl}ethanamine |
| 25 | 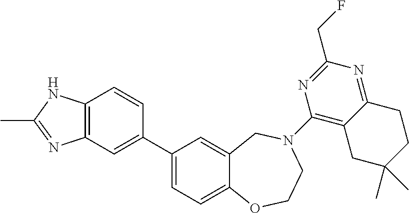 | 4-[2-(fluoromethyl)-6,6- dimethyl-5,6,7,8- tetrahydroquinazolin-4-yl]-7- (2-methyl-1H-benzimidazol-5- yl)-2,3,4,5-tetrahydro-1,4- benzoxazepine |
| 26 | 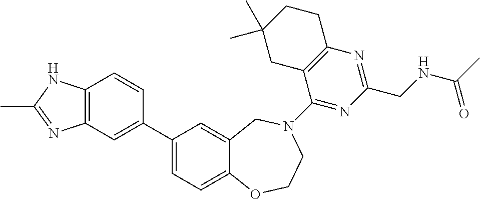 [SMILES: CC(=O)NCc1nc2c(c(N3CCOc4ccc(-c5ccc6[nH]c(C)nc6c5)cc4C3)n1)CC(C)(C)CC2] | N-({6,6-dimethyl-4-[7-(2- methyl-1H-benzimidazol-5-yl)- 2,3-dihydro-1,4-benzoxazepin- 4(5H)-yl]-5,6,7,8- tetrahydroquinazolin-2- yl}methyl)acetamide |
| 27 | 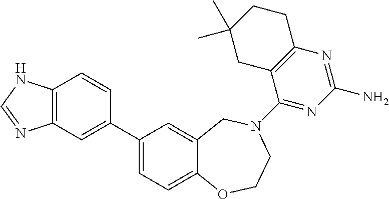 [SMILES: CC1(C)CCc2nc(N)nc(N3CCOc4ccc(-c5ccc6[nH]cnc6c5)cc4C3)c2C1] | 4-[7-(1H-benzimidazol-5-yl)- 2,3-dihydro-1,4-benzoxazepin- 4(5H)-yl]-6,6-dimethyl- 5,6,7,8-tetrahydroquinazolin-2- amine |
| 28 | 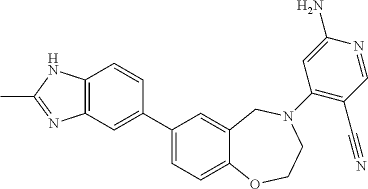 [SMILES: Cc1nc2cc(-c3ccc4c(c3)CN(c3cc(N)ncc3C#N)CCO4)ccc2[nH]1] | 6-amino-4-[7-(2-methyl-1H- benzimidazol-5-yl)-2,3- dihydro-1,4-benzoxazepin- 4(5H)-yl]pyridine-3- carbonitrile |
| 29 | 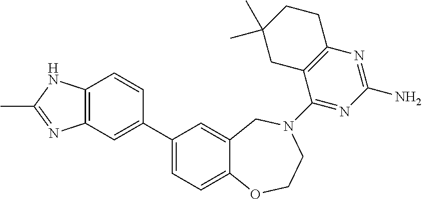 [SMILES: Cc1nc2cc(-c3ccc4c(c3)CN(c3nc(N)nc5c3CC(C)(C)CC5)CCO4)ccc2[nH]1] | 6,6-dimethyl-4-[7-(2-methyl- 1H-benzimidazol-5-yl)-2,3- dihydro-1,4-benzoxazepin- 4(5H)-yl]-5,6,7,8- tetrahydroquinazolin-2-amine |
| 30 | 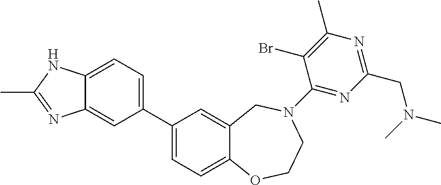 | 1-{5-bromo-4-methyl-6-[7-(2- methyl-1H-benzimidazol-5-yl)- 2,3-dihydro-1,4-benzoxazepin- 4(5H)-yl]pyrimidin-2-yl}-N,N- dimethylmethanamine |
| 31 | 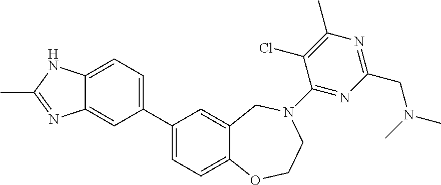 | 1-{5-chloro-4-methyl-6-[7-(2- methyl-1H-benzimidazol-5-yl)- 2,3-dihydro-1,4-benzoxazepin- 4(5H)-yl]pyrimidin-2-yl}-N,N- dimethylmethanamine |
| 32 | 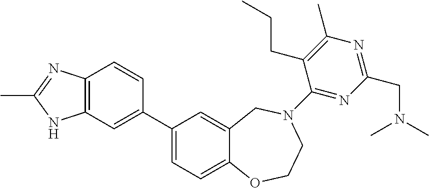 | N,N-dimethyl-1-{4-methyl-6- [7-(2-methyl-1H- benzimidazol-6-yl)-2,3- dihydro-1,4-benzoxazepin- 4(5H)-yl]-5-propylpyrimidin- 2-yl}methanamine |
| 33 | 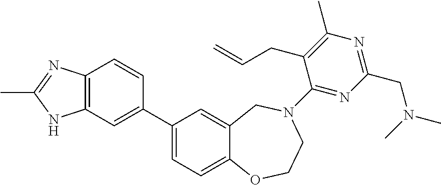 | N,N-dimethyl-1-{4-methyl-6- [7-(2-methyl-1H- benzimidazol-6-yl)-2,3- dihydro-1,4-benzoxazepin- 4(5H)-yl]-5-prop-2-en-1- ylpyrimidin-2-yl}methanamine |
| 34 | 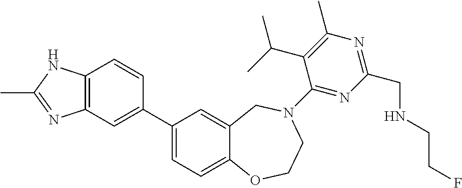 [SMILES: Cc1nc2cc(-c3ccc4c(c3)CN(c3nc(CNCCF)nc(C)c3C(C)C)CCO4)ccc2[nH]1] | 2-fluoro-N-({4-methyl-6-[7-(2- methyl-1H-benzimidazol-5-yl)- 2,3-dihydro-1,4-benzoxazepin- 4(5H)-yl]-5-(1- methylethyl)pyrimidin-2- yl}methyl)ethanamine |
| 35 | 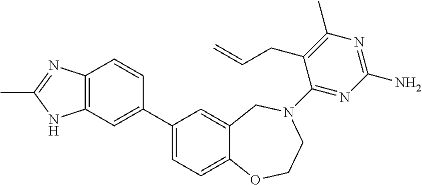 [SMILES: C=CCc1c(C)nc(N)nc1N1CCOc2ccc(-c3ccc4nc(C)[nH]c4c3)cc2C1] | 4-methyl-6-[7-(2-methyl-1H- benzimidazol-5-yl)-2,3- dihydro-1,4-benzoxazepin- 4(5H)-yl]-5-prop-2-en-1- ylpyrimidin-2-amine |
| 36 | 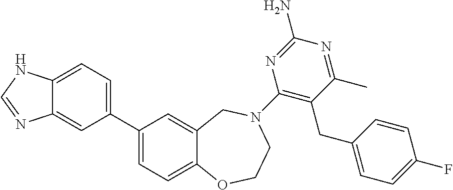 [SMILES: Cc1nc(N)nc(N2CCOc3ccc(-c4ccc5[nH]cnc5c4)cc3C2)c1Cc1ccc(F)cc1] | 4-[7-(1H-benzimidazol-5-yl)- 2,3-dihydro-1,4-benzoxazepin- 4(5H)-yl]-5-[(4- fluorophenyl)methyl]-6- methylpyrimidin-2-amine |
| 37 | 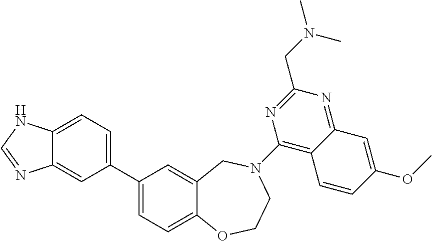 [SMILES: COc1ccc2c(N3CCOc4ccc(-c5ccc6[nH]cnc6c5)cc4C3)nc(CN(C)C)nc2c1] | 1-{4-[7-(1H-benzimidazol-5- yl)-2,3-dihydro-1,4- benzoxazepin-4(5H)-yl]-7- (methyloxy)quinazolin-2-yl}- N,N-dimethylmethanamine |
| 38 | 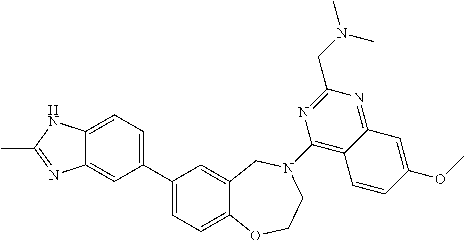 | N,N-dimethyl-1-{4-[7-(2- methyl-1H-benzimidazol-5-yl)- 2,3-dihydro-1,4-benzoxazepin- 4(5H)-yl]-7- (methyloxy)quinazolin-2- yl}methanamine |
| 39 | 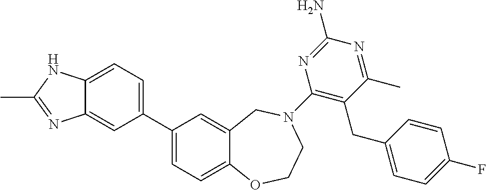 [SMILES: Cc1nc2cc(-c3ccc4c(c3)CN(c3nc(N)nc(C)c3Cc3ccc(F)cc3)CCO4)ccc2[nH]1] | 5-[(4-fluorophenyl)methyl]-4- methyl-6-[7-(2-methyl-1H- benzimidazol-5-yl)-2,3- dihydro-1,4-benzoxazepin- 4(5H)-yl]pyrimidin-2-amine |
| 40 | 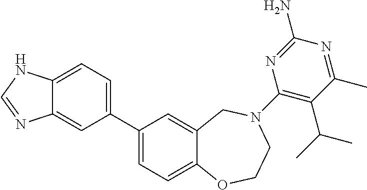 [SMILES: Cc1nc(N)nc(N2CCOc3ccc(-c4ccc5[nH]cnc5c4)cc3C2)c1C(C)C] | 4-[7-(1H-benzimidazol-5-yl)- 2,3-dihydro-1,4-benzoxazepin- 4(5H)-yl]-6-methyl-5-(1- methylethyl)pyrimidin-2-amine |
| 41 | 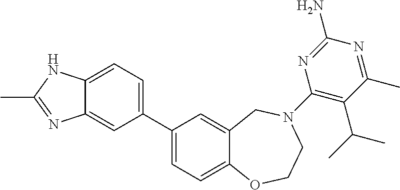 | 4-methyl-6-[7-(2-methyl-1H- benzimidazol-5-yl)-2,3- dihydro-1,4-benzoxazepin- 4(5H)-yl]-5-(1- methylethyl)pyrimidin-2-amine |
| 42 | 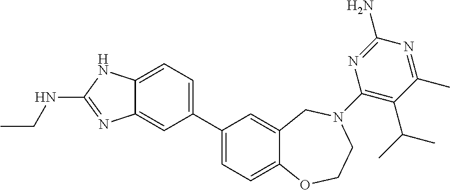 [SMILES: CCNc1nc2cc(-c3ccc4c(c3)CN(c3nc(N)nc(C)c3C(C)C)CCO4)ccc2[nH]1] | 5-{4-[2-amino-6-methyl-5-(1- methylethyl)pyrimidin-4-yl]- 2,3,4,5-tetrahydro-1,4- benzoxazepin-7-yl}-N-ethyl- 1H-benzimidazol-2-amine |
| 43 | 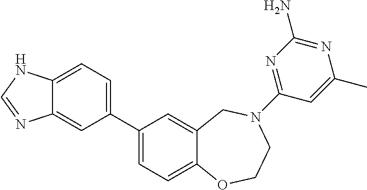 [SMILES: Cc1cc(N2CCOc3ccc(-c4ccc5[nH]cnc5c4)cc3C2)nc(N)n1] | 4-[7-(1H-benzimidazol-5-yl)- 2,3-dihydro-1,4-benzoxazepin- 4(5H)-yl]-6-methylpyrimidin- 2-amine |
| 44 | 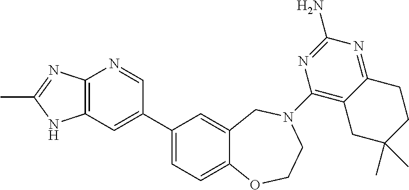 | 6,6-dimethyl-4-[7-(2-methyl- 1H-imidazo[4,5-b]pyridin-6- yl)-2,3-dihydro-1,4- benzoxazepin-4(5H)-yl]- 5,6,7,8-tetrahydroquinazolin-2- amine |
| 45 | 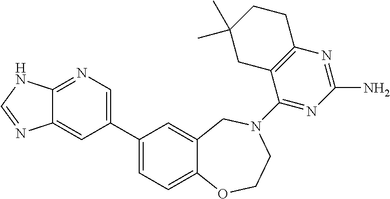 [SMILES: CC1(C)CCc2nc(N)nc(N3CCOc4ccc(-c5cnc6[nH]cnc6c5)cc4C3)c2C1] | 4-[7-(3H-imidazo[4,5- b]pyridin-6-yl)-2,3-dihydro- 1,4-benzoxazepin-4(5H)-yl]- 6,6-dimethyl-5,6,7,8- tetrahydroquinazolin-2-amine |
| 46 | 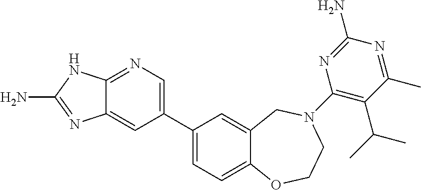 [SMILES: Cc1nc(N)nc(N2CCOc3ccc(-c4cnc5[nH]c(N)nc5c4)cc3C2)c1C(C)C] | 6-{4-[2-amino-6-methyl-5-(1- methylethyl)pyrimidin-4-yl]- 2,3,4,5-tetrahydro-1,4- benzoxazepin-7-yl}-3H- imidazo[4,5-b]pyridin-2-amine |
| 47 | 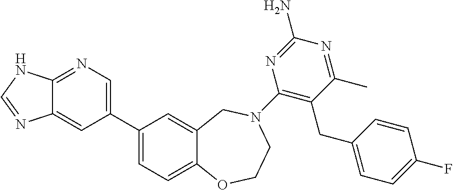 [SMILES: Cc1nc(N)nc(N2CCOc3ccc(-c4cnc5[nH]cnc5c4)cc3C2)c1Cc1ccc(F)cc1] | 5-[(4-fluorophenyl)methyl]-4- [7-(3H-imidazo[4,5-b]pyridin- 6-yl)-2,3-dihydro-1,4- benzoxazepin-4(5H)-yl]-6- methylpyrimidin-2-amine |
| 48 | 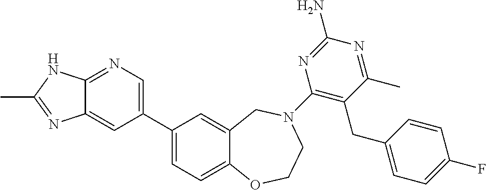 [SMILES: Cc1nc2cc(-c3ccc4c(c3)CN(c3nc(N)nc(C)c3Cc3ccc(F)cc3)CCO4)cnc2[nH]1] | 5-[(4-fluorophenyl)methyl]-4- methyl-6-[7-(2-methyl-3H- imidazo[4,5-b]pyridin-6-yl)- 2,3-dihydro-1,4-benzoxazepin- 4(5H)-yl]pyrimidin-2-amine |
| 49 | 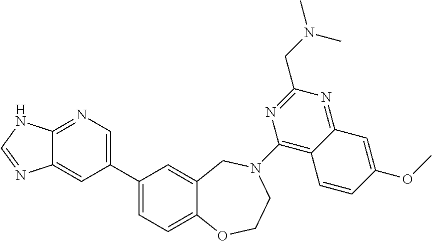 [SMILES: COc1ccc2c(N3CCOc4ccc(-c5cnc6[nH]cnc6c5)cc4C3)nc(CN(C)C)nc2c1] | 1-{4-[7-(3H-imidazo[4,5- b]pyridin-6-yl)-2,3-dihydro- 1,4-benzoxazepin-4(5H)-yl]-7- (methyloxy)quinazolin-2-yl}- N,N-dimethylmethanamine |
| 50 | 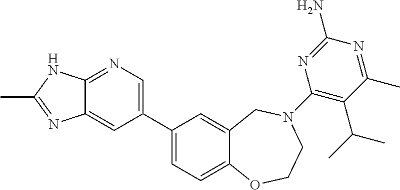 | 4-methyl-5-(1-methylethyl)-6- [7-(2-methyl-3H-imidazo[4,5- b]pyridin-6-yl)-2,3-dihydro- 1,4-benzoxazepin-4(5H)- yl]pyrimidin-2-amine |
| 51 | 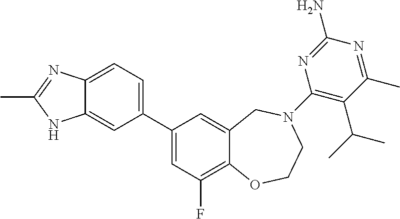 | 4-[9-fluoro-7-(2-methyl-1H- benzimidazol-6-yl)-2,3- dihydro-1,4-benzoxazepin- 4(5H)-yl]-6-methyl-5-(1- methylethyl)pyrimidin-2-amine |
| 52 | 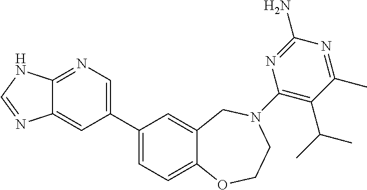 [SMILES: Cc1nc(N)nc(N2CCOc3ccc(-c4cnc5[nH]cnc5c4)cc3C2)c1C(C)C] | 4-[7-(3H-imidazo[4,5- b]pyridin-6-yl)-2,3-dihydro- 1,4-benzoxazepin-4(5H)-yl]-6- methyl-5-(1-methylethyl) pyrimidine-2-amine |

### General Administration

In one aspect, the invention provides pharmaceutical compositions comprising an inhibitor of PI3K/mTOR according to the invention and a pharmaceutically acceptable carrier, excipient, or diluent. In certain other specific embodiments, administration is by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, specifically in unit dosage forms suitable for simple administration of precise dosages.

The compositions will include a conventional pharmaceutical carrier or excipient and a compound of the invention as the/an active agent, and, in addition, may include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Compressed gases may be used to disperse a compound of this invention in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts or solvates, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

If formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate.

### General Synthesis

Compounds of this invention can be made by the synthetic procedures described below. The starting materials and reagents used in preparing these compounds are either available from commercial suppliers such as Aldrich Chemical Co. (Milwaukee, Wis.), or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4^{th} Edition) and Larock's Comprehensive Organic Transformations (VCH Publishers Inc., 1989). These examples are merely illustrative of some methods by which the compounds of this invention can be synthesized, and various modifications to these examples can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure and over a temperature range from about −78° C. to about 150° C., more specifically from about 0° C. to about 125° C. and more specifically at about room (or ambient) temperature, e.g., about 20° C. Unless otherwise stated (as in the case of an hydrogenation), all reactions are performed under an atmosphere of nitrogen.

Prodrugs can be prepared by techniques known to one skilled in the art. These techniques generally modify appropriate functional groups in a given compound. These modified functional groups regenerate original functional groups by routine manipulation or in vivo. Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

The compounds of the invention, or their pharmaceutically acceptable salts, may have asymmetric carbon atoms or quaternized nitrogen atoms in their structure. Compounds of the Invention that may be prepared through the syntheses described herein may exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention.

Some of the compounds of the invention may contain an active ketone —C(O)CF₃ and may exist in part or in whole as the —C(OH₂)CF₃ form. Regardless of whether the compound is drawn as the —C(O)CF₃ or —C(OH₂)CF₃ form, both are included within the scope of the Invention. Although an individual compound may be drawn as the —C(O)CF₃ form, one of ordinary skill in the art would understand that the compound may exist in part or in whole as the —C(OH₂)CF₃ form and that the ratio of the two forms may vary depending on the compound and the conditions in which it exists.

Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention.

The present invention also includes N-oxide derivatives and protected derivatives of compounds of the Invention. For example, when compounds of the Invention contain an oxidizable nitrogen atom, the nitrogen atom can be converted to an N-oxide by methods well known in the art. When compounds of the Invention contain groups such as hydroxy, carboxy, thiol or any group containing a nitrogen atom(s), these groups can be protected with a suitable “protecting group” or “protective group”. A comprehensive list of suitable protective groups can be found in T. W. Greene, Protective Groups in Organic Synthesis, John Wiley & Sons, Inc. 1991, the disclosure of which is incorporated herein by reference in its entirety. The protected derivatives of compounds of the Invention can be prepared by methods well known in the art.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The chemistry for the preparation of the compounds of this invention is known to those skilled in the art. In fact, there may be more than one process to prepare the compounds of the invention. The following examples illustrate but do not limit the invention. All references cited herein are incorporated by reference in their entirety.

### SYNTHETIC EXAMPLES

### 4-Chloro-5-isopropyl-6-methylpyrimidin-2-amine

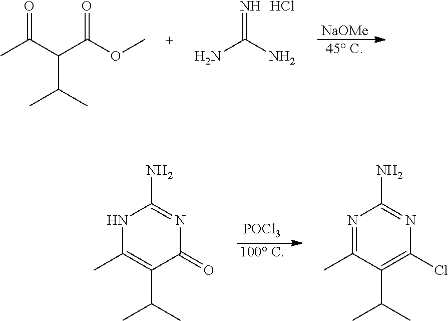

### Step 1:

To a solution of ethyl 2-isopropylacetoacetate (22.0 g, 0.18 mol) and guanidine hydrochloride (18.0 g, 0.19 mol) in methanol (100 mL) was added sodium methoxide (0.38 mol, 86.4 mL, 25% methanol solution) at 0° C. via dropping funnel over 30 min. The reaction mixture was allowed to room temperature then heated to 50° C. for 18 hrs. The mixture was concentrated, diluted with ethyl acetate (20 mL) and adjusted to pH 6-7 with 6N aqueous hydrochloric acid. The resulting solid was filtered and washed with water. The filtrates were concentrated and repeated filtration afforded a second crop of solid. The combined solids were dried under vacuum to give 2-amino-5-isopropyl-6-methylpyrimidin-4(1M-one as a pale yellow solid (16.8 g, 56%); ¹H NMR (400 MHz, DMSO-d₆): δ 10.5 (s, 1H), 6.17 (s, 2H), 2.85 (m, 1H), 2.03 (s, 3H), 1.15 (d, 6H); MS (EI) for C₈H₁₃N₃O: 168.2 (MH⁺).

### Step 2:

To a solution of 2-amino-5-isopropyl-6-methylpyrimidin-4(1H)-one (4.93 g, 29.5 mmol) in phosphorus oxychloride (50 mL) was refluxed for 18 hrs. The reaction mixture was concentrated and the residue partitioned with a mixture if ethyl acetate and water (10 mL each). The biphasic mixture was quenched with solid sodium bicarbonate addition until the aqueous phase pH was 6-7. The aqueous layer was extracted with ethyl acetate (3×100 mL) and the combined organic solutions dried over magnesium sulfate, filtered and concentrated to afford 4-chloro-5-isopropyl-6-methylpyrimidin-2-amine as a pale brown solid (4.92 g, 90%); ¹H NMR (400 MHz, DMSO-d₆): δ 6.71 (s, 2H), 3.26 (m, 1H), 3.25 (s, 3H), 1.21 (d, 6H); MS (EI) for C₈H₁₂ClN₃: 186.1 (MH⁺).

### 1-(4-Chloro-6,6-dimethyl-5,6,7,8-tetrahydroquinazolin-2-yl)-N,N-dimethylmethanamine

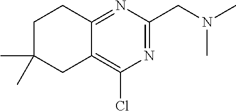

### Step 1:

To a solution of methyl 5,5-dimethyl-2-oxocyclohexanecarboxylate (6.0 g, 33 mmol) and 2-chloroacetimidamide hydrochloride (4.6 g, 36 mmol) in methanol (30 mL) was added sodium methoxide (4.4 M in MeOH, 9.0 mL, 40 mmol). The reaction mixture was stirred at ambient temperature for three hours and then concentrated. The resulting residue was partitioned between ethyl acetate and aqueous sodium bicarbonate. The organic layer was washed with brine, dried over magnesium sulfate and concentrated. Purification by silica gel chromatography provided 2-(chloromethyl)-6,6-dimethyl-5,6,7,8-tetrahydroquinazolin-4-ol (4.2 g, 57% yield) as a white solid. MS (ES) for C₁₁H₁₅ClN₂O: 227 (MH⁺).

### Step 2:

To a solution of 2-(chloromethyl)-6,6-dimethyl-5,6,7,8-tetrahydroquinazolin-4-ol (2.5 g, 11 mmol) in THF (10 mL) was added dimethylamine (2M in THF, 16.5 mL, 33 mmol). The reaction mixture was heated (60° C.) for two hours and then partitioned between ethyl acetate and sodium bicarbonate. The organic layer was washed with brine, dried over magnesium sulfate, filtered and concentrated to provide 2-((dimethylamino)methyl)-6,6-dimethyl-5,6,7,8-tetrahydroquinazolin-4-ol, which was used in step 3 without further purification. MS (ES) for C₁₃H₂₁N₃O: 236 (MH⁺).

### Step 3:

To a solution of the final residue from step 2 in CHCl₃ (10 mL) was added POCl₃ (10 mL). The reaction mixture was heated (90° C.) for two hours and concentrated. This residue was partitioned between dichloromethane and aqueous sodium bicarbonate and the resulting organic layer was washed with brine, dried over magnesium sulfate, filtered and concentrated in vacuo. Purification by silica gel chromatography (5-10% concentrated aqueous ammonia in methanol) in chloroform provided 1-(4-chloro-6,6-dimethyl-5,6,7,8-tetrahydroquinazolin-2-yl)-N,N-dimethylmethanamine (1.3 g, 48% yield). ¹H NMR (400 MHz, CD₃OD) δ 4.52 (s, 2H), 3.02 (s, 6H), 2.98 (t, 2H), 2.61 (s, 2H), 1.71 (t, 2H), 1.06 (s, 6H); MS (ES) for C₁₃H₂₀ClN₃: 254 (MH⁺).

Using analogous synthetic techniques and substituting dimethylamine in step 2 with cesium acetate, (4-chloro-6,6-dimethyl-5,6,7,8-tetrahydroquinazolin-2-yl)methyl acetate was prepared:

### 1,1-Dimethylethyl 7-bromo-2,3-dihydro-1,4-benzoxazepine-4(5H)-carboxylate

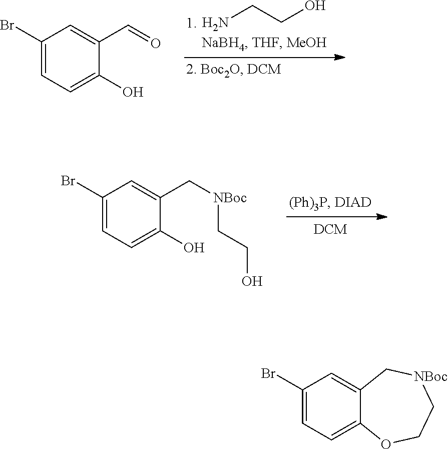

### Step 1:

Commercially-available 5-bromo-2-hydroxybenzaldehyde (4.0 g, 10 mmol) and 2-aminoethanol were combined in THF/MeOH (100 mL, 10:1) and sodium borohydride (0.76 g, 2.0 mmol) was added with stirring. The resulting reaction mixture was stirred at 40° C. for 4 h, concentrated on a rotary evaporator then diluted with EtOAc (50 mL) and saturated NaHCO₃ (30 mL). To this suspension was added di-tert-butyl dicarbonate (2.83 g, 13 mmol). The mixture was stirred at rt overnight. The organic layer was washed with water, dried over anhydrous magnesium sulfate, filtered, and concentrated on a rotary evaporator. Hexane was subsequently added to the crude reaction product which resulted in the formation of a white solid. This slurry was filtered to obtain tert-butyl-5-bromo-2-hydroxybenzyl(2-hydroxyethyl)carbamate (6.8 g, 98%) as a white solid. MS (EI) for C₁₄H₂₀BrNO₄. found 346 (MH⁺).

### Step 2:

tert-Butyl-5-bromo-2-hydroxybenzyl(2-hydroxyethyl)carbamate (3.46 g, 10 mmol) and triphenylphosphine (3.96 g, 15 mmol) were combined in DCM (100 mL) and diisopropyl azodicarboxylate (3.03 g, 15 mmol) was added. The resulting reaction mixture was stirred at rt for 12 h. The reaction mixture was washed with water, dried, filtered, and concentrated on a rotary evaporator. The resulting crude product was purified via silica gel chromatography eluting with 8:2 hexane/ethyl acetate to give the desired product (1.74 g, 53%) as a white solid. MS (EI) for C₁₄H₁₈BrNO₃. found 328 (MH⁺).

### (4-{[(1,1-Dimethylethyl)oxy]carbonyl}-2,3,4,5-tetrahydro-1,4-benzoxazepin-7-yl)boronic acid

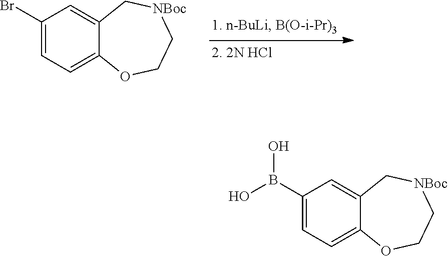 [SMILES: CC(C)(C)OC(=O)N1CCOc2ccc(Br)cc2C1 ; CC(C)(C)OC(=O)N1CCOc2ccc(B(O)O)cc2C1]

### Step 1:

A solution of 1,1-dimethylethyl 7-bromo-2,3-dihydro-1,4-benzoxazepine-4(5H)-carboxylate (30.0 g, 91.4 mmol) and triisopropyl borate (22.4 g, 119 mmol) in THF (300 mL) was cooled to −78° C., and a 2.5 M solution of n-butyllithium in hexanes (47.6 mL, 119 mmol) was added dropwise over 40 min at this temperature. The reaction mixture was stirred at −78° C. for an additional 30 min, then quenched by dropwise addition of 2 N hydrochloric acid (80 ml), and allowed to warm up to room temperature. Ethyl acetate (100 mL) and water (100 mL) were added, the organic layer was separated, and the aqueous layer was extracted with ethyl acetate (100 mL). The combined organic layers were washed with water, dried over sodium sulfate, and concentrated. Hexane (200 mL) was added to the residue and the mixture was stirred overnight. The precipitate was filtered, washed several times with hexane, and dried to give (4-{[(1,1-dimethylethyl)oxy]carbonyl}-2,3,4,5-tetrahydro-1,4-benzoxazepin-7-yl)boronic acid (23.4 g, 87%) as a colorless solid. MS (EI) for C₁₄H₂₀BNO₅: 294 (MH⁺).

### 7-Bromo-2,3,4,5-tetrahydro-1,4-benzoxazepine hydrochloride salt

### Step 1:

1,1-Dimethylethyl 7-bromo-2,3-dihydro-1,4-benzoxazepine-4(5H)-carboxylate (10 g, 30.5 mmol) was taken into hot ethanol (10 mL) followed by addition of 4 M hydrogen chloride in dioxane solution (2.1 eq, 16 mL) and the resulting solution was allowed to slowly cool to ambient temperature over one hour. An excess of ethyl ether was then added and the resulting slurry was filtered. The filter cake was washed with ethyl ether and dried to give 7-bromo-2,3,4,5-tetrahydro-1,4-benzoxazepine hydrochloride (7.9 g, 98% yield) as a colorless crystalline solid. MS (EI) for C₉H₁₀NOBr: 229 (MH⁺).

### Example 1

### 4-Methyl-6-[7-(2-methyl-1H-benzimidazol-5-yl)-2,3-dihydro-1,4-benzoxazepin-4(5H)-yl]-5-(1-methylethyl)pyrimidin-2-amine

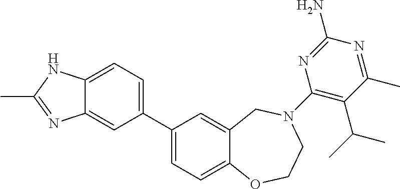

### Step 1:

To 5-bromo-2-methylbenzimidazole (38 g, 180 mmol) in THF (400 mL) was added di-tert-butyl dicarbonate (39 g, 189 mmol). The reaction mixture was stirred at room temperature for 24 h and then concentrated. Ethyl acetate (400 mL) was added to the residue, and the solution was washed with 10% aqueous citric acid (2×100 mL), water (100 mL), and brine (100 mL), dried over sodium sulfate, and concentrated. Column chromatography on silica (gradient 20-30% ethyl acetate in hexane) provided 1,1-dimethylethyl 6-bromo-2-methyl-1H-benzimidazole-1-carboxylate (27 g, 48% yield) as a beige solid. MS (EI) for C₁₃H₁₅BrN₂O₂: 312 (MH⁺).

### Step 2:

A suspension of 1,1-dimethylethyl 6-bromo-2-methyl-1H-benzimidazole-1-carboxylate (11.3 g, 36 mmol), (4-{[(1,1-dimethylethyl)oxy]carbonyl}-2,3,4,5-tetrahydro-1,4-benzoxazepin-7-yl)boronic acid (11.7 g, 40 mmol), dichloro[1,1-bis(diphenyl-phosphino]ferrocenepalladium (II) dichloromethane adduct (3.0 g, 10 mol %) in dioxane (115 mL) and water (28.5 mL) was degassed with nitrogen, and then diisopropylethylamine (18.6 g, 144 mmol) was added. The reaction mixture was stirred at 90° C. for 220 min, cooled to room temperature, and concentrated. Column chromatography on silica of the residue (gradient 25-30% ethyl acetate in hexane) afforded 1,1-dimethylethyl 7-(1-{[(1,1-dimethylethyl)oxy]carbonyl}-2-methyl-1H-benzimidazol-6-yl)-2,3-dihydro-1,4-benzoxazepine-4(5H)-carboxylate (13.2 g, 76% yield) as an amorphous solid. MS (EI) for C₂₇H₃₃N₃O₅: 480 (MH⁺).

### Step 3:

A solution of 1,1-dimethylethyl 7-(1-{[(1,1-dimethylethyl)oxy]carbonyl}-2-methyl-1H-benzimidazol-6-yl)-2,3-dihydro-1,4-benzoxazepine-4(5H)-carboxylate (13.1 g, 27 mmol) in a mixture of methanol (20 mL) and 4 N hydrogen chloride in dioxane (30 mL) was refluxed for 15 min. After cooling to room temperature ethyl ether (100 mL) was added, and the reaction mixture was concentrated. Another portion of ethyl ether (100 mL) was added, the precipitate was filtered off, washed several times with ethyl ether, and dried to give 7-(2-methyl-1H-benzimidazol-6-yl)-2,3,4,5-tetrahydro-1,4-benzoxazepine dihydrochloride (8.9 g, 93% yield) as a light beige solid. ¹HNMR (400 MHz, CD₃OD): □7.93 (s, 1H), 7.86-7.67 (m, 4H), 7.28 (s, 1H), 4.54 (s, 2H), 4.33-4.23 (m, 2H), 3.65-3.54 (m, 2H), 2.91 (s, 3H); MS (EI) for C₁₇H₁₇N₃O: 280 (MH⁺).

### Step 4:

To a solution of 742-methyl-1H-benzimidazol-6-yl)-2,3,4,5-tetrahydro-1,4-benzoxazepine dihydrochloride (137 mg, 0.39 mmol) and 4-chloro-5-isopropyl-6-methylpyrimidin-2-amine (72.5 mg, 0.39 mmol) in NMP (2 mL) was added diisopropylethylamine (0.56 mL, 0.28 mmol) at room temperature. The reaction mixture was heated to 100° C. for 5 h. The reaction was cooled to room temperature and diluted with water, then extracted with ethyl acetate (20 mL×3). The combined extracts were dried over magnesium sulfate, filtered and concentrated under reduced pressure. The resulting residue was purified by preparative reverse phase HPLC using an aqueous ammonium acetate buffered water/acetonitrile mobile phase and the combined pure fractions lyophilized to give 4-methyl-6-[7-(2-methyl-1H-benzimidazol-5-yl)-2,3-dihydro-1,4-benzoxazepin-4(5H)-yl]-5-(1-methylethyl)pyrimidin-2-amine (62.3 mg, 37%). ¹H NMR (400 MHz, DMSO-d₆): δ 7.63 (s, 1H), 7.51 (d, 3H), 7.36 (d, 1H), 7.05 (dd, 1H), 6.02 (s, 2H), 4.25 (s, 4H), 3.54 (s, 2H), 3.24 (m, 1H), 2.28 (d, 3H), 1.27 (d, 6H); MS (EI) for C₂₅H₂₈N₆O: 429.3 (MH⁺).

Proceeding according to the method of Example 1 and replacing 4-chloro-5-isopropyl-6-methylpyrimidin-2-amine with alternative reagents in step 4, the following compounds of the invention were prepared:

### 1-{6,6-Dimethyl-4-[7-(2-methyl-1H-benzimidazol-5-yl)-2,3-dihydro-1,4-benzoxazepin-4(5H)-yl]-5,6,7,8-tetrahydroquinazolin-2-yl}ethanamine

¹H NMR (400 MHz, MeOH-d₄): 7.98 (s, 1H), 7.86 (d, 1H), 7.84 (s, 1H), 7.81 (s, 1H), 7.79 (s, 1H), 7.62 (dd, 1H), 7.10 (d, 1H), 5.23 (br s, 2H), 4.60 (m, 1H), 4.57 (m, 2H), 4.35 (m, 2H), 3.34 (s, 2H), 2.91 (m, 2H), 2.87 (s, 3H), 2.60 (br s, 2H), 1.69 (m, 2H), 1.66 (d, 3H), 0.89 (s, 6H); MS (EI) for C₂₉H₃₄N₆O: 483 (MH⁺).

### N,N-Dimethyl-1-{4-methyl-6-[7-(2-methyl-1H-benzimidazol-6-yl)-2,3-dihydro-1,4-benzoxazepin-4(5H)-yl]-5-[2-(methyloxy)ethyl]pyrimidin-2-yl}methanamine

¹H NMR (400 MHz, DMSO-d₆): δ 12.25 (s, 1H), 7.67 (m, 2H), 7.47 (dd, 2H), 7.39 (d, 1H), 7.01 (d, 1H), 4.60 (s, 2H), 4.29 (m, 2H), 3.79 (m, 2H), 3.53 (t, 2H), 3.18 (m, 4H), 2.92 (t, 2H), 2.36 (s, 3H), 2.13 (s, 6H); MS (EI) for C₂₈H₃₄N₆O₂: 487.3 (MH⁺).

### 1-{4,5-Dimethyl-6-[7-(2-methyl-1H-benzimidazol-6-yl)-2,3-dihydro-1,4-benzoxazepin-4(5H)-yl]pyrimidin-2-yl}-N,N-dimethylmethanamine

¹H NMR (400 MHz, DMSO-d₆): δ 7.61 (m, 2H), 7.45 (dd, 2H), 7.37 (d, 1H), 7.00 (d, 1H), 4.61 (s, 2H), 4.29 (m, 2H), 3.82 (m, 2H), 3.39 (s, 2H), 2.31 (s, 3H), 2.19 (s, 3H), 2.17 (s, 6H), 1.91 (s, 3H-OAc peak); MS (EI) for C₂₆H₃₀N₆O: 443.3 (MH⁺).

### N-Ethyl-N-({4-methyl-6-[7-(2-methyl-1H-benzimidazol-5-yl)-2,3-dihydro-1,4-benzoxazepin-4(5H)-yl]-5-(1-methylethyl)pyrimidin-2-yl}methyl)ethanamine

¹H NMR (400 MHz, DMSO-d₆): δ 7.53 (m, 4H), 7.35 (d, 1H), 7.03 (d, 1H), 4.43 (s, 2H), 4.30 (m, 2H), 3.68 (m, 2H), 3.59 (s, 2H), 2.51 (m partially buried, 5H), 2.48 (s, 3H), 1.91 (s, 3H), 1.32 (d, 6H), 0.94 (t, 6H); MS (EI) for C₃₀H₃₈N₆O: 499.3 (MH⁺).

### {4-Methyl-6-[7-(2-methyl-1H-benzimidazol-5-yl)-2,3-dihydro-1,4-benzoxazepin-4(5H)-yl]-5-(1-methylethyl)pyrimidin-2-yl}methyl acetate

¹H NMR (400 MHz, DMSO-d₆ plus D₂O): δ 7.70 (s, 1 μl), 7.56 (m, 3H), 7.37 (m, 1H), 7.05 (d, 1H), 4.95 (s, 2H), 4.42 (s, 2H), 4.28 (m, 2H), 3.68 (m, 2H), 3.32-3.24 (m, 1H), 2.49 (s, 3H), 2.05 (s, 3H), 1.31 (d, 6H); MS (EI) for C₂₈H₃₁N₅O₃: 486.3 (MH⁺).

### {4-Methyl-6-[7-(2-methyl-1H-benzimidazol-5-yl)-2,3-dihydro-1,4-benzoxazepin-4(5H)-yl]-5-(1-methylethyl)pyrimidin-2-yl}methanol

¹H NMR (400 MHz, DMSO-d₆ plus D₂O): δ 7.55-7.42 (m, 4H), 7.36 (d, 1H), 7.01 (d, 1H), 4.80 (br t, 0.5H—OH peak), 4.43 (s, 2H), 4.32 (s, 2H), 4.26 (m, 2H), 3.72 (m, 2H), 3.25 (q, 1H), 2.47 (s partially buried, 3H), 2.44 (s, 3H), 1.29 (d, 6H); MS (EI) for C₂₆H₂₉N₅O₂: 444.3 (MH⁺).

### Methyl {2-[(dimethylamino)methyl]-4-methyl-6-[7-(2-methyl-1H-benzimidazol-5-yl)-2,3-dihydro-1,4-benzoxazepin-4(5H)-yl]pyrimidin-5-yl}acetate

¹H NMR (400 MHz, DMSO-d₆): δ 7.67-7.41 (m, 3H), 7.38-7.27 (m, 3H), 6.97 (d, 1H), 4.48 (d, 2H), 4.27 (m, 2H), 3.74 (m, 2H), 3.68 (s, 2H), 3.63 (s, 2H), 3.40 (s buried, 6H), 2.21 (s, 3H), 2.11 (s, 6H), 1.89 (s, 3H—OAc peak); MS (EI) for C₂₈H₃₂N₆O₃: 501.3 (MH⁺).

### N,N-Dimethyl-1-{4-methyl-6-[7-(2-methyl-1H-benzimidazol-5-yl)-2,3-dihydro-1,4-benzoxazepin-4(5H)-yl]-5-phenylpyrimidin-2-yl}methanamine

¹H NMR (400 MHz, DMSO-d₆): 7.50 (ddd, 4H), 7.43-7.29 (m, 5H), 7.23 (d, 1H), 6.92 (dd, 1H), 4.51 (d, 2H), 4.10-3.96 (m, 2H), 3.53 (d, 2H), 3.38 (d, 2H), 2.50 (s, 3H), 2.16 (d, 6H), 2.02 (d, 3H); MS (EI) for C₃₁H₃₂N₆O: 505.0 (MH⁺).

### 7-(2-Methyl-1H-benzimidazol-5-yl)-4-[6-methyl-5-(1-methylethyl)-2-(pyrrolidin-1-ylmethyl)pyrimidin-4-yl]-2,3,4,5-tetrahydro-1,4-benzoxazepine

¹H NMR (400 MHz, DMSO-d₆): 7.60 (s, 1H), 7.53-7.39 (m, 3H), 7.33 (dd, 1H), 7.00 (d, 1H), 4.40 (s, 2H), 4.27 (d, 2H), 3.65 (s, 2H), 3.47 (s, 2H), 3.33-3.22 (m, 1H), 2.47 (s, 3H), 2.44 (d, 3H), 2.41 (s, 4H), 1.85 (s, 6H, OAc), 1.56 (dt, 4H), 1.30 (d, 6H); MS (EI) for C₃₀H₃₆N₆O: 497.2 (MH⁺).

### 1-{5-(3-Fluoropropyl)-4-methyl-6-[7-(2-methyl-1H-benzimidazol-5-yl)-2,3-dihydro-1,4-benzoxazepin-4(5H)-yl]pyrimidin-2-yl}-N,N-dimethylmethanamine

¹H NMR (400 MHz, DMSO-d₆): 7.75 (d, 2H), 7.63 (d, 1H), 7.53 (dd, 2H), 7.01 (d, 1H), 4.80 (s, 2H), 4.40 (s, 2H), 4.34 (t, 1H), 4.29 (s, 2H), 4.22 (t, 1H), 3.87 (s, 2H), 2.79 (d, 2H), 2.77 (s, 6H), 2.62 (s, 3H), 2.42 (s, 3H), 1.81 (m, 1H), 1.79 (m, 1H); MS (E1) for C₂₈H₃₃FN₆O: 489.3 (MH⁺).

### N,N-Dimethyl-1-[4-methyl-6-[7-(2-methyl-1H-benzimidazol-5-yl)-2,3-dihydro-1,4-benzoxazepin-4(5H)-yl]-5-(2-methylpropyl)pyrimidin-2-yl]methanamine

¹H NMR (400 MHz, DMSO-d₆): 12.22 (s, 1H), 7.62 (m, 2H), 7.54 (s, 1H), 7.42 (dd, 1H), 7.34 (d, 1H), 6.96 (d, 1H), 4.56 (s, 2H), 4.29 (s, 2H), 3.73 (s, 2H), 3.30 (s, 2H, overlapped), 2.57 (d, 2H), 2.46 (s, 3H, overlapped), 2.32 (s, 3H), 2.10 (s, 6H), 1.88 (s, 2H, OAc), 1.75-1.56 (m, 1H), 0.52 (d, 7H); MS (EI) for C₂₉H₃₆N₆O: 485 (MH⁺).

### 6-Amino-4-[7-(2-methyl-1H-benzimidazol-5-yl)-2,3-dihydro-1,4-benzoxazepin-4(5H)-yl]pyridine-3-carbonitrile

¹H NMR (400 MHz, d₆-DMSO) δ 8.37 (d, 1H), 7.87 (d, 2H), 7.84 (s, 1H), 7.65 (s, 1H), 7.55 (dd, 1H), 7.06 (d, 1H), 5.90 (bs, 2H), 4.50 (m, 2H), 4.30 (s, 2H), 3.76 (m, 2H), 2.55 (s, 2H), 1.91 (d, 3H), 1.90 (m, 1H), 1.23 (d, 2H), 0.93 (s, 6H); MS (ES) for C₂₃H₂₀N₆O: 397 (MH⁺).

### 6,6-Dimethyl-4-[7-(2-methyl-1H-benzimidazol-5-yl)-2,3-dihydro-1,4-benzoxazepin-4(5H)-yl]-5,6,7,8-tetrahydroquinazolin-2-amine

¹H NMR (400 MHz, CD₃OD) δ 7.91 (s, 1H), 7.81 (d, 1H), 7.76 (d, 1H), 7.72 (d, 1H), 7.56 (dd, 1H), 7.07 (d, 1H), 5.02 (s, 2H), 4.42 (m, 2H), 4.19 (m, 2H), 2.86 (s, 3H), 2.67 (t, 2H), 2.41 (s, 2H), 1.61 (t, 2H), 0.89 (s, 61-1); MS (ES) for C₂₇H₃₀N₆O: 455 (MH⁺).

### 1-{5-Bromo-4-methyl-6-[7-(2-methyl-1H-benzimidazol-5-yl)-2,3-dihydro-1,4-benzoxazepin-4(5H)-yl]pyrimidin-2-yl}-N,N-dimethylmethanamine

¹H NMR (400 MHz, methanol-d₄): 7.63 (m, 2H), 7.52 (d, 1H), 7.43 (m, 2H), 7.00 (d, 1H), 5.06 (s, 2H), 4.35 (m, 2H), 4.18 (m, 2H), 3.79 (s, 2H), 2.58 (s, 3H), 2.56 (s, 3H), 2.47 (s, 6H), 1.93 (s, 6H); MS (EI) for C₂₅H₂₇BrN₆O: 507/509 (MH⁺).

### 1-{5-Chloro-4-methyl-6-[7-(2-methyl-1H-benzimidazol-5-yl)-2,3-dihydro-1,4-benzoxazepin-4(5H)-yl]pyrimidin-2-yl}-N,N-dimethylmethanamine

¹H NMR (400 MHz, methanol-d₄): 7.62 (m, 2H), 7.52 (d, 1H), 7.43 (m, 2H), 7.02 (d, 1H), 5.08 (s, 2H), 4.34 (m, 2H), 4.23 (m, 2H), 3.84 (s, 2H), 2.58 (s, 3H), 2.52 (s, 6H), 2.50 (s, 3H), 1.94 (s, 3H); MS (EI) for C₂₅H₂₇ClN₆O: 463 (MH⁺).

### N,N-Dimethyl-1-{4-methyl-6-[7-(2-methyl-1H-benzimidazol-6-yl)-2,3-dihydro-1,4-benzoxazepin-4(5H)-yl]-5-propylpyrimidin-2-yl}methanamine

¹H NMR (400 MHz, DMSO-d₆): 7.61 (m, 2H), 7.46 (m, 2H), 7.36 (d, 1H), 6.99 (d, 1H), 4.61 (s, 2H), 4.32 (s, 2H), 3.78 (s, 2H), 3.34 (br s, 2H), 2.61-2.52 (m, 2H), 2.51 (s, 3H), 2.35 (s, 3H), 2.10 (s, 6H), 1.45 (m, 2H), 0.75 (t, 3H); MS (EI) for C₂₈H₃₄N₆O: 471 (MH⁺).

### N,N-Dimethyl-1-{4-methyl-6-[7-(2-methyl-1H-benzimidazol-6-yl)-2,3-dihydro-1,4-benzoxazepin-4(5H)-yl]-5-prop-2-en-1-ylpyrimidin-2-yl}methanamine

¹H NMR (400 MHz, DMSO-d₆): 7.63 (s, 1H), 7.56-7.43 (m, 3H), 7.36 (d, 1H), 7.00 (d, 1H), 6.13 (m, 1H), 5.27 (d, 1H), 4.95 (d, 1H), 4.61 (s, 2H), 4.27 (br s, 2H), 3.82 (s, 2H), 3.37 (s, 2H), 3.32 (s, 2H), 2.50 (s, 3H), 2.27 (s, 3H), 2.18 (s, 6H); MS (EI) for C₂₈H₃₂N₆O: 469 (MH⁺).

### 2-Fluoro-N-({4-methyl-6-[7-(2-methyl-1H-benzimidazol-5-yl)-2,3-dihydro-1,4-benzoxazepin-4(5H)-yl]-5-(1-methylethyl)pyrimidin-2-yl}methyl)ethanamine

¹H NMR (400 MHz, DMSO-d₆): 7.63 (s, 1H), 7.55 (s, 1H), 7.50 (d, 2H), 7.37 (d, 1H), 7.02 (d, 1H), 4.45 (m, 3H), 4.35-4.20 (m, 3H), 3.71 (s, 2H), 3.63 (s, 2H), 3.34-3.21 (m, 1H), 2.76 (t, 1H), 2.69 (t, 1H), 2.47 (s, 3H), 1.87 (s, 3H), 1.33 (d, 6H); MS (EI) for C₂₈H₃₃FN₆O: 489 (MH⁺).

### 4-Methyl-6-[7-(2-methyl-1H-benzimidazol-5-yl)-2,3-dihydro-1,4-benzoxazepin-4(5H)-yl]-5-prop-2-en-1-ylpyrimidin-2-amine

¹H NMR (400 MHz, DMSO-d₆): 12.23 (s, 1H), 7.67-7.49 (m, 4H), 7.35 (s, 1H), 7.03 (d, 1H), 6.11 (m, 1H), 6.00 (s, 2H), 5.26 (m, 1H), 5.00 (d, 1H), 4.44 (s, 2H), 4.26 (s, 2H), 3.68 (s, 2H), 3.18 (s, 2H), 2.49 (s, 3H), 2.11 (s, 3H); MS (E1) for C₂₅H₂₆N₆O: 427 (MH⁺).

### N,N-Dimethyl-1-{4-[7-(2-methyl-1H-benzimidazol-5-yl)-2,3-dihydro-1,4-benzoxazepin-4(5H)-yl]-7-(methyloxy)quinazolin-2-yl}methanamine

¹H NMR (400 MHz, DMSO-d₆): δ 7.94 (t, 1H), 7.68 (s, 2H), 7.42 (dd, 3H), 7.16 (s, 1H), 7.08 (d, 1H), 6.95 (t, 1H), 5.03 (d, 2H), 4.43 (d, 2H), 4.17 (d, 2H), 3.89 (s, 3H), 3.44 (s, 2H), 2.50 (s, 3H), 2.14 (s, 6H); MS (EI) for C₂₉H₃₀N₆O₂: 495.2 (MH⁺).

### 5-[(4-Fluorophenyl)methyl]-4-methyl-6-[7-(2-methyl-1H-benzimidazol-5-yl)-2,3-dihydro-1,4-benzoxazepin-4(5H)-yl]pyrimidin-2-amine

¹H NMR (400 MHz, DMSO-d₆): δ 7.62-7.38 (m, 3H), 7.26-7.06 (m, 6H), 7.00 (d, 1H), 6.84 (s, 1H), 6.45 (s, 2H), 4.40 (s, 2H), 4.22 (s, 2H), 3.85 (s, 2H), 3.71 (s, 2H), 2.52 (d, 3H), 2.01 (s, 3H); MS (EI) for C₂₉H₂₇FN₆O: 495.3 (MH⁺).

Proceeding according to the method of Example 1 and replacing 5-bromo-2-methylbenzimidazole with 5-bromobenzimidazole in step 1 and 4-chloro-5-isopropyl-6-methylpyrimidin-2-amine with alternative reagents in step 4 the following compounds of the invention were prepared:

### 4-[7-(1H-Benzimidazol-5-yl)-2,3-dihydro-1,4-benzoxazepin-4(5H)-yl]-6,6-dimethyl-5,6,7,8-tetrahydroquinazolin-2-amine

¹H NMR (400 MHz, CD₃OD) δ 8.19 (s, 1H), 7.80 (d, 1H), 7.65 (d, 1H), 7.59 (d, 1H), 7.53 (dd, 1H), 7.50 (m, 1H), 7.05 (d, 1H), 4.79 (s, 2H), 4.35 (m, 2H), 4.01 (m, 2H), 2.63 (t, 2H), 2.41 (s, 2H), 1.61 (t, 2H), 0.90 (s, 6H); MS (ES) for C₂₆H₂₈N₆O: 441 (MH⁺).

### 4-[7-(1H-Benzimidazol-5-yl)-2,3-dihydro-1,4-benzoxazepin-4(5H)-yl]-5-[(4-fluorophenyl)methyl]-6-methylpyrimidin-2-amine

¹H NMR (400 MHz, DMSO-d₆): δ 8.24 (d, 1H), 7.92-7.50 (m, 1H), 7.47 (d, 1H), 7.20 (d, 1H), 7.12 (dq, 5H), 7.02 (d, 1H), 6.75 (d, 1H), 6.11 (d, 2H), 4.31 (s, 2H), 4.20 (d, 2H), 3.82 (d, 2H), 3.65 (s, 2H), 1.98 (s, 3H); MS (EI) for C₂₈H₂₅FN₆O: 481.0 (MH⁺).

### 1-{4-[7-(1H-Benzimidazol-5-yl)-2,3-dihydro-1,4-benzoxazepin-4(5H)-yl]-7-(methyloxy)quinazolin-2-yl}-N,N-dimethylmethanamine

¹H NMR (400 MHz, DMSO-d₆): δ 8.25 (s, 1H), 7.95 (d, 1H), 7.81 (s, 1H), 7.74-7.62 (m, 2H), 7.49 (d, 2H), 7.16 (s, 1H), 7.10-7.03 (m, 1H), 6.98 (d, 1H), 5.05 (s, 2H), 4.45 (s, 2H), 4.20 (s, 2H), 3.87 (d, 3H), 3.47 (s, 2H), 2.13 (d, 6H); MS (EI) for C₂₈H₂₈N₆O₂:481.3 (MH⁺).

### 4-[7-(1H-Benzimidazol-5-yl)-2,3-dihydro-1,4-benzoxazepin-4(5H)-yl]-6-methyl-5-(1-methylethyl)pyrimidin-2-amine

¹H NMR (400 MHz, DMSO-d₆): δ 8.20 (d, 1H), 7.82-7.70 (m, 2H), 7.61 (d, 1H), 7.57-7.48 (m, 2H), 7.45 (d, 1H), 7.06 (t, 1H), 6.02 (s, 2H), 4.23 (d, 4H), 3.51 (d, 2H), 3.22 (d, 1H), 2.30 (s, 3H), 1.26 (t, 6H); MS (EI) for C₂₄H₂₆N₆O: 415.3 (MH⁺).

### 4-[7-(1H-benzimidazol-5-yl)-2,3-dihydro-1,4-benzoxazepin-4(5H)-yl]-6-methylpyrimidin-2-amine

¹H NMR (400 MHz, DMSO-d₆): δ 8.22 (s, 1H), 7.81 (s, 2H), 7.62 (s, 1H), 7.45 (dd, 2H), 7.00 (d, 1H), 6.09 (s, 1H), 5.94 (s, 2H), 4.73 (s, 2H), 4.03 (dd, 4H), 2.03 (d, 3H), 1.88 (s, 6H); MS (EI) for C₂₁H₂₀N₆O: 373.2 (MH⁺).

Proceeding according to the method of Example 1 and replacing 7-(2-methyl-1H-benzimidazol-6-yl)-2,3,4,5-tetrahydro-1,4-benzoxazepine dihydrochloride with N-ethyl-5-(2,3,4,5-tetrahydro-1,4-benzoxazepin-7-yl)-1H-benzimidazol-2-amine in step 4 the following compounds of the invention were prepared:

### 5-{4-[2-Amino-6-methyl-5-(1-methylethyl)pyrimidin-4-yl]-2,3,4,5-tetrahydro-1,4-benzoxazepin-7-yl}-N-ethyl-1H-benzimidazol-2-amine

¹H NMR (400 MHz, DMSO-d₆): δ 7.44 (d, 3H), 7.32 (s, 1H), 7.29-7.02 (m, 2H), 7.04 (d, 1H), 6.60 (s, 1H), 6.02 (s, 2H), 6.02 (s, 27H), 4.23 (s, 4H), 3.54 (s, 2H), 3.50 (d, 2H), 3.23 (d, 1H), 2.51 (s, 3H), 2.28 (d, 2H), 1.27 (d, 6H), 1.18 (t, 3H); MS (EI) for C₂₆H₃₁N₇O: 458.3 (MH⁺).

### Example 2

### 1-{4-[7-{3-[(Difluoromethyl)oxy]-4-(methyloxy)phenyl}-2,3-dihydro-1,4-benzoxazepin-4(5H)-yl]-6,6-dimethyl-5,6,7,8-tetrahydroquinazolin-2-yl}-N,N-dimethylmethanamine

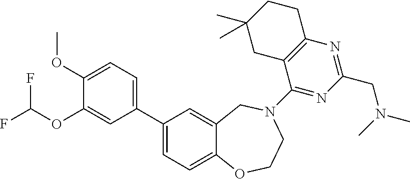 [SMILES: COc1ccc(-c2ccc3c(c2)CN(c2nc(CN(C)C)nc4c2CC(C)(C)CC4)CCO3)cc1OC(F)F]

### Step 1:

A mixture of (4-{[(1,1-dimethylethyl)oxy]carbonyl}-2,3,4,5-tetrahydro-1,4-benzoxazepin-7-yl)boronic acid (123 mg, 0.42 mmol), 4-bromo-2-[(difluoromethyl)oxy]-1-(methyloxy)benzene (106 mg, 0.42 mmol), potassium carbonate (116 mg, 0.84 mmol), 1,1′-bis(diphenylphosphino)ferrocene-palladium(II)dichloride dichloromethane complex (17 mg, 0.021 mmol) in dioxane (800 uL) and water (200 uL) was sparged with nitrogen gas for 2 min. The mixture was then heated in a microwave reactor at 110° C. for 15 min. The mixture was then diluted with water and extracted three times with ethyl acetate. The organic extracts were combined, dried over sodium sulfate, filtered, and concentrated in vacuo. The residue was purified by silica gel gradient chromatography (100% hexanes to 50% ethyl acetate in hexanes) to provide 1,1-dimethylethyl 7-{3-[(difluoromethyl)oxy]-4-(methyloxy)phenyl}-2,3-dihydro-1,4-benzoxazepine-4(5H)-carboxylate (102.4 mg, 0.243 mmol, 58% yield) as a colorless syrup. ¹H NMR (400 MHz, CDCl₃) δ 7.50-7.30 (m, 3H), 7.12-6.97 (m, 2H), 6.59 (t, 1H), 4.59-4.43 (m, 2H), 4.09-4.05 (m, 2H), 3.92 (s, 3H), 3.85-3.75 (m, 2H), 1.46-1.37 (m, 9H); MS (EI) for C₂₂H₂₅F₂NO₅: 322 (MH⁺-Boc).

### Step 2:

To a solution of 1,1-dimethylethyl 7-{3-[(difluoromethyl)oxy]-4-(methyloxy)phenyl}-2,3-dihydro-1,4-benzoxazepine-4(5H)-carboxylate (102 mg, 0.24 mmol) in methanol (2 mL) was added anhydrous hydrogen chloride in dioxane (4 M, 600 uL, 2.4 mmol), and the resulting solution was heated to 60° C. for 40 min. After cooling to rt the mixture was concentrated and the residue dried in vacuo to provide 7-{3-[(difluoromethyl)oxy]-4-(methyloxy)phenyl}-2,3,4,5-tetrahydro-1,4-benzoxazepine hydrochloride salt in quantitative yield. ¹H NMR (400 MHz, DMSO-d₆) δ 9.32 (s, 2H), 7.79 (d, 1H), 7.64 (dd, 1H), 7.55 (dd, 1H), 7.46 (d, 1H), 7.36-6.95 (m, 3H), 4.40 (s, 2H), 4.21 (s, 2H), 3.87 (s, 3H), 3.50 (s, 2H); MS (EI) for C₁₇H₁₇F₂NO₃: 322 (MH⁺).

### Step 3:

A solution of 7-{3-[(difluoromethyl)oxy]-4-(methyloxy)phenyl}-2,3,4,5-tetrahydro-1,4-benzoxazepine hydrochloride salt (86 mg, 0.24 mmol), 1-(4-chloro-6,6-dimethyl-5,6,7,8-tetrahydroquinazolin-2-yl)-N,N-dimethylmethanamine (61 mg, 0.24 mmol), and diisopropylethylamine (209 uL, 1.2 mmol) in NMP (500 μL) was heated to 120° C. for 23 h and was then cooled to rt. Water was then added and the resulting aqueous mixture was extracted twice with ethyl acetate. The combined organic extracts were dried over sodium sulfate, filtered, and concentrated in vacuo. The residue was purified by preparative reverse phase HPLC to provide 1-{4-[7-{3-[(difluoromethyl)oxy]-4-(methyloxy)phenyl}-2,3-dihydro-1,4-benzoxazepin-4(5H)-yl]-6,6-dimethyl-5,6,7,8-tetrahydroquinazolin-2-yl}-N,N-dimethylmethanamine (15.1 mg, 0.028 mmol, 12% yield) as white solid. ¹H NMR (400 MHz, DMSO-d₆) δ 7.67 (d, 1H), 7.54 (dd, 1H), 7.50-7.43 (m, 2H), 7.24 (d, 1H), 7.13 (t, 1H), 7.00 (d, 1H), 4.71 (s, 2H), 4.37-4.29 (m, 2H), 3.94-3.88 (m, 2H), 3.87 (s, 3H), 2.72 (s, 2H), 2.58 (br s, 6H), 2.47 (s, 2H), 1.61 (t, 2H), 0.85 (s, 6H); MS (EI) for C₃₀H₃₆F₂N₄O₃: 539 (MH⁺).

Proceeding according to the method of Example 2 and replacing 4-bromo-2-[(difluoromethyl)oxy]-1-(methyloxy)benzene in step 1 and/or 1-(4-chloro-6,6-dimethyl-5,6,7,8-tetrahydroquinazolin-2-yl)-N,N-dimethylmethanamine in step 3 with alternative reagents, the following compounds of the invention were prepared:

### 1-(6,6-Dimethyl-4-{7-[4-(methyloxy)-3-[{2-(methyloxy)ethyl]oxy}phenyl]-2,3-dihydro-1,4-benzoxazepin-4(5H)-yl}-5,6,7,8-tetrahydroquinazolin-2-yl)-N,N-dimethylmethanamine

¹H NMR (400 MHz, DMSO-d₆) δ 7.61 (d, 1H), 7.44 (dd, 1H), 7.20-7.14 (m, 2H), 7.02 (d, 1H), 6.97 (d, 1H), 4.60 (s, 2H), 4.31-4.24 (m, 2H), 4.18-4.13 (m, 2H), 3.87-3.82 (m, 2H), 3.79 (s, 3H), 3.70-3.66 (m, 2H), 3.33 (s, 3H), 2.69 (t, 2H), 2.45 (s, 2H), 2.14 (s, 6H), 1.59 (t, 2H), 0.86 (s, 6H); MS (EI) for C₃₂H₄₂N₄O₄: 547 (MH⁺).

### 1-(6,6-Dimethyl-4-{7-[4-(methyloxy)-3-(methylsulfonyl)phenyl]-2,3-dihydro-1,4-benzoxazepin-4(5H)-yl}-5,6,7,8-tetrahydroquinazolin-2-yl)-N,N-dimethylmethanamine

¹H NMR (400 MHz, DMSO-d₆) δ 8.00 (d, 1H), 7.96 (dd, 1H), 7.63 (d, 1H), 7.45 (dd, 1H), 7.40 (d, 1H), 7.01 (d, 1H), 4.66 (s, 2H), 4.36-4.25 (m, 2H), 4.00 (s, 3H), 3.91-3.78 (m, 2H), 3.41 (s, 2H), 3.28 (s, 3H), 2.68 (t, 2H), 2.43 (s, 2H), 2.12 (br s, 6H), 1.59 (t, 2H), 0.85 (s, 6H); MS (E1) for C₃₀H₃₈N₄O₄S: 551 (MH⁺).

### N-[5-(4-{2-[(Dimethylamino)methyl]-6-methyl-5-(1-methylethyl)pyrimidin-4-yl}-2,3,4,5-tetrahydro-1,4-benzoxazepin-7-yl)-2-(methyloxy)phenyl]methanesulfonamide

¹H NMR (400 MHz, DMSO-d₆) δ 9.03 (s, 1H), 7.51 (d, 1H), 7.49-7.39 (m, 3H), 7.17 (d, 1H), 7.02 (d, 1H), 4.45 (s, 2H), 4.33-4.26 (m, 2H), 3.87 (s, 3H), 3.74-3.66 (m, 2H), 3.32 (s, 2H), 3.30-3.21 (m, 1H), 2.98 (s, 3H), 2.48 (s, 3H), 2.30 (br s, 6H), 1.32 (d, 6H); MS (EI) for C₂₈H₃₇N₅O₄S: 540 (MH⁺).

### N-[5-{4-[2-Amino-6-methyl-5-(1-methylethyl)pyrimidin-4-yl]-2,3,4,5-tetrahydro-1,4-benzoxazepin-7-yl}-2-(methyloxy)phenyl]methanesulfonamide

¹H NMR (400 MHz, DMSO-d₆) δ 7.41 (d, 1H), 7.36-7.29 (m, 2H), 7.01 (d, 1H), 6.71 (d, 1H), 6.65 (d, 1H), 5.99 (s, 2H), 4.20 (M, 4H), 3.66 (s, 3H), 3.57-3.49 (m, 2H), 3.28-3.18 (m, 1H), 2.55 (s, 3H), 2.30 (s, 3H), 1.27 (d, 6H); MS (EI) for C₂₅H₃₁N₅O₄S: 498 (MH⁺).

### 1-[5-(4-{2-[(Dimethylamino)methyl]-6,6-dimethyl-5,6,7,8-tetrahydroquinazolin-4-yl}-2,3,4,5-tetrahydro-1,4-benzoxazepin-7-yl)-2-(methyloxy)phenyl]ethanone

¹H NMR (400 MHz, DMSO-d₆) δ 7.85-7.78 (m, 2H), 7.62 (d, 1H), 7.44 (dd, 1H), 7.26 (d, 1H), 6.99 (d, 1H), 4.62 (s, 2H), 4.33-4.25 (m, 2H), 3.93 (s, 3H), 3.89-3.80 (m, 2H), 3.39 (s, 2H), 2.69 (t, 2H), 2.56 (s, 2H), 2.44 (s, 3H), 2.15 (s, 6H), 1.59 (t, 2H), 0.86 (s, 6H); MS (EI) for C₃₁H₃₈N₄O₃: 515 (MH⁺).

### Example 3

### N-Azetidin-3-yl-4-methyl-6-[7-(2-methyl-1H-benzimidazol-5-yl)-2,3-dihydro-1,4-benzoxazepin-4(5H)-yl]-5-(1-methylethyl)pyrimidin-2-amine

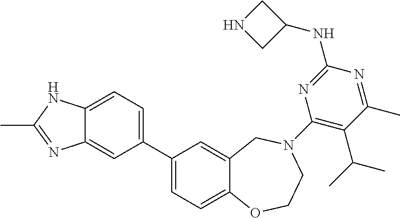

### Step 1:

Sodium metal (167 mg, 7 mmol) was dissolved in ethanol (20 mL) to form ethanolic sodium ethoxide. To that solution was added thiourea (265 mg, 3.5 mmol) and ethyl 2-acetyl-3-methylbutanoate (540 uL, 2.9 mmol). After stirring at reflux for 2 h, the reaction mixture was cooled to rt and then concentrated in vacuo. The residue was diluted with water and then acidified to pH 7 with acetic acid. A white precipitate formed, which was collected by filtration and dried in vacuo to provide 6-methyl-5-(1-methylethyl)-2-thioxo-2,3-dihydropyrimidin-4(1H)-one (354 mg, 1.92 mmol, 66% yield) as a white crystalline solid. ¹H NMR (400 MHz, DMSO-d₆) δ 12.20 (br s, 1H), 11.97 (br s, 1H), 2.88-2.78 (m, 1H), 2.13 (s, 3H), 1.16 (d, 6H); MS (EI) for C₉H₁₂N₂OS: 185 (MH⁺).

### Step 2:

A solution of 6-methyl-5-(1-methylethyl)-2-thioxo-2,3-dihydropyrimidin-4(1H)-one (120 mg, 0.64 mmol) in DMF (1 mL) was treated with iodomethane (80 uL, 1.28 mmol) for 2 h at rt. The mixture was then diluted with 10% aqueous lithium chloride and extracted twice with ethyl acetate. The combined organic extracts were washed with 10% aqueous lithium chloride, dried over sodium sulfate, filtered, and concentrated in vacuo to provide 6-methyl-5-(1-methylethyl)-2-(methylthio)pyrimidin-4(3H)-one (126.8 mg, 0.64 mmol, 100% yield) as an off-white solid. ¹H NMR (400 MHz, CDCl₃) δ 12.19 (br s, 1H), 3.07-2.97 (m, 1H), 2.56 (s, 3H), 2.32 (s, 3H), 1.31 (d, 6H); MS (EI) for C₉H₁₄N₂OS: 199 (MH⁺).

### Step 3:

To a solution of 6-methyl-5-(1-methylethyl)-2-(methylthio)pyrimidin-4(3H)-one (127 mg, 0.64 mmol) in chloroform (1 mL) was added phosphorous oxychloride (1 mL), and the resulting mixture was stirred at 70° C. for 1 h. After cooling to rt, the volatile materials were removed in vacuo. The residue was then diluted with dichloromethane. The organic solution was washed with saturated aqueous sodium bicarbonate, and the aqueous phase was back extracted with dichloromethane. The combined organic extracts were dried over sodium sulfate, filtered, and concentrated in vacuo to provide 4-chloro-6-methyl-5-(1-methylethyl)-2-(methylthio)pyrimidine (121.3 mmol, 0.56 mmol, 87% yield) as a yellow oil. ¹H NMR (400 MHz, CDCl₃) δ 3.51-3.39 (m, 1H), 2.55 (s, 3H), 2.54 (s, 3H), 1.36 (d, 6H); MS (EI) for C₉H₁₃ClN₂S: 217, 219 (Cl isotopes, MH⁺).

### Step 4:

A solution of 7-(2-methyl-1H-benzimidazol-5-yl)-2,3,4,5-tetrahydro-1,4-benzoxazepine hydrochloride salt (177 mg, 0.56 mmol), 4-chloro-6-methyl-5-(1-methylethyl)-2-(methylthio)pyrimidine (121 mg, 0.56 mmol), and diisopropylethylamine (490 uL, 2.8 mmol) in DMA (800 uL) was heated to 110° C. for 21 h and was then cooled to rt. 10% aqueous lithium chloride was then added, and the resulting aqueous mixture was extracted twice with ethyl acetate. The combined organic extracts were dried over sodium sulfate, filtered, and concentrated in vacuo. The residue was then purified by gradient silica gel chromatography (100% dichloromethane to 10% methanol in dichloromethane) to provide 7-(2-methyl-1H-benzimidazol-5-yl)-4-[6-methyl-5-(1-methylethyl)-2-(methylthio)pyrimidin-4-yl]-2,3,4,5-tetrahydro-1,4-benzoxazepine (145 mg, 0.315 mmol, 56% yield) as a yellow syrup. ¹H NMR (400 MHz, CDCl₃) δ 7.58 (br m, 1H), 7.45 (dd, 1H), 7.43 (d, 1H), 7.40 (dd, 1H), 7.08 (d, 1H), 4.42 (s, 2H), 4.34-4.28 (m, 2H), 3.81-3.76 (m, 2H), 3.37-3.27 (m, 1H), 2.65 (s, 3H), 2.48 (s, 3H), 2.41 (s, 3H), 1.34 (d, 6H); MS (EI) for C₂₆H₂₉N₅OS: 460 (MH⁺).

### Step 5:

A solution of 7-(2-methyl-1H-benzimidazol-5-yl)-4-[6-methyl-5-(1-methylethyl)-2-(methylthio)pyrimidin-4-yl]-2,3,4,5-tetrahydro-1,4-benzoxazepine (145 mg, 0.315 mmol) in dichloromethane (2.5 mL) was treated with 3-chloroperbenzoic acid (136 mg, 0.788 mmol). After stirring 50 min, the mixture was diluted with dichloromethane and washed with saturated aqueous sodium bicarbonate. The aqueous phase was back extracted with dichloromethane. The combined organic extracts were washed with saturated aqueous sodium bicarbonate, dried over sodium sulfate, filtered, and concentrated in vacuo to provide 7-(2-methyl-1H-benzimidazol-5-yl)-4-[6-methyl-5-(1-methylethyl)-2-(methylsulfonyl)pyrimidin-4-yl]-2,3,4,5-tetrahydro-1,4-benzoxazepine in quantitative yield as a yellow/orange solid. ¹H NMR (400 MHz, CDCl₃) δ 7.71 (s, 1H), 7.58 (d, 1H), 7.51 (d, 1H), 7.48-7.37 (m, 2H), 7.05 (d, 1H), 4.60 (s, 2H), 4.39-4.24 (m, 2H), 3.95-3.85 (m, 2H), 3.41-3.26 (m, 1H), 3.03 (s, 3H), 2.66 (s, 3H), 2.64 (s, 3H), 1.40 (d, 6H); MS (EI) for C₂₆H₂₉N₅O₃S: 492 (MH⁺).

### Step 6:

To a solution of 7-(2-methyl-1H-benzimidazol-5-yl)-4-[6-methyl-5-(1-methylethyl)-2-(methylsulfonyl)pyrimidin-4-yl]-2,3,4,5-tetrahydro-1,4-benzoxazepine (70.3 mg, 0.143 mmol) in NMP (250 uL) was added 1,1-dimethylethyl 3-aminoazetidine-1-carboxylate (123 mg, 0.715 mmol), and the mixture was stirred at 145° C. for 20 h. After cooling to rt, 10% aqueous lithium chloride was added. The resulting aqueous solution was extracted twice with ethyl acetate. The combined organic extracts were washed with 10% aqueous lithium chloride, dried over sodium sulfate, filtered, and concentrated in vacuo. The residue was purified by preparative reverse phase HPLC to provide 1,1-dimethylethyl 3-({4-methyl-6-[7-(2-methyl-1H-benzimidazol-5-yl)-2,3-dihydro-1,4-benzoxazepin-4(5H)-yl]-5-(1-methylethyl)pyrimidin-2-yl}amino)azetidine-1-carboxylate (17.4 mg, 0.030 mmol, 21% yield) as an orange film. ¹H NMR (400 MHz, CD₃OD) δ 7.67 (s, 1H), 7.57-7.42 (m, 4H), 7.01 (d, 1H), 4.50 (s, 2H), 4.45-4.36 (m, 1H), 4.31-4.26 (m, 2H), 3.98 (t, 2H), 3.76-3.71 (m, 2H), 3.68 (dd, 2H), 3.29-3.23 (m, 1H), 2.57 (s, 3H), 2.36 (s, 3H), 1.38-1.31 (m, 15H); MS (EI) for C₃₃H₄₁N₇O₃: 584 (MH⁺).

### Step 7:

To a solution of 1,1-dimethylethyl 3-({4-methyl-6-[7-(2-methyl-1H-benzimidazol-5-yl)-2,3-dihydro-1,4-benzoxazepin-4(5H)-yl]-5-(1-methylethyl)pyrimidin-2-yl}amino)azetidine-1-carboxylate (17.4 mg, 0.030 mmol) in methanol 200 uL) was added anhydrous hydrogen chloride in dioxane (4 N, 75 uL, 0.3 mmol), and the resulting mixture was heated to 60° C. for 25 min. After cooling to rt, the volatile materials were removed in vacuo to provide N-azetidin-3-yl-4-methyl-6-[7-(2-methyl-1H-benzimidazol-5-yl)-2,3-dihydro-1,4-benzoxazepin-4(5H)-yl]-5-(1-methylethyl)pyrimidin-2-amine dihydrochloride salt (16.2 mg) as a beige solid. ¹H NMR (400 MHz, CD₃OD) δ 7.93 (s, 1H), 7.86-7.78 (m, 2H), 7.68 (d, 1H), 7.59 (dd, 1H), 7.08 (d, 1H), 4.97-4.89 (m, 3H), 4.57-4.52 (m, 2H), 4.31-4.24 (m, 2H), 4.18-4.11 (m, 2H), 4.09-4.03 (m, 2H), 3.09-2.99 (m, 1H), 2.88 (s, 3H), 2.50 (s, 3H), 1.34 (d, 6H); MS (EI) for C₂₈H₃₃N₇O: 484 (MH⁺).

Proceeding according to the method of example 3 and replacement of ethyl 2-acetyl-3-methylbutanoate with ethyl 2-(4-fluorobenzyl)-3-oxobutanoate in step 1 and/or 1,1-dimethylethyl 3-aminoazetidine-1-carboxylate with alternative amines in step 6 the following compounds of the invention were prepared:

### N′-{5-[(4-Fluorophenyl)methyl]-4-methyl-6-[7-(2-methyl-1H-benzimidazol-5-yl)-2,3-dihydro-1,4-benzoxazepin-4(5H)-yl]pyrimidin-2-yl}-N,N-dimethylethane-1,2-diamine

¹H NMR (400 MHz, DMSO-d₆) δ 12.22 (d, 1H), 7.62-7.49 (m, 1H), 7.47-7.39 (m, 2H), 7.21-7.06 (m, 5H), 6.98 (dd, 1H), 6.92-6.80 (m, 1H), 6.38-6.25 (m, 1H), 4.40 (br s, 2H), 4.26-4.18 (m, 2H), 3.86 (s, 2H), 3.72-3.63 (m, 2H), 3.29-3.19 (m, 2H), 2.51 (s, 3H), 2.36-2.24 (m, 2H), 2.12 (br s, 6H), 1.99 (s, 3H); MS (EI) for C₃₃H₃₆FN₇O: 566 (MH⁺).

### N-Ethyl-5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-amine

¹H NMR (400 MHz, CD₃OD) δ 7.59-7.46 (m, 2H), 7.41 (dd, 1H), 7.26 (d, 1H), 7.17-7.08 (m, 2H), 7.01-6.93 (m, 3H), 6.90 (s, 1H), 4.50 (s, 2H), 4.27-4.20 (m, 2H), 3.90 (s, 2H), 3.85-3.75 (m, 2H), 3.31-3.23 (m, 2H), 2.60 (s, 3H), 2.04 (s, 3H), 1.05 (t, 3H); MS (EI) for C₃₁H₃₁FN₆O: 523 (MH⁺).

### N′-{5-[(4-Fluorophenyl)methyl]-4-methyl-6-[7-(2-methyl-1H-benzimidazol-5-yl)-2,3-dihydro-1,4-benzoxazepin-4(5H)-yl]pyrimidin-2-yl}-N,N-dimethylpropane-1,3-diamine

¹H NMR (400 MHz, DMSO-d₆) δ 12.22 (d, 1H), 7.66-7.48 (m, 1H), 7.48-7.36 (m, 2H), 7.23-7.05 (m, 5H), 7.02-6.87 (m, 2H), 6.64-6.51 (m, 1H), 4.42 (br s, 2H), 4.26-4.16 (m, 2H), 3.86 (s, 2H), 3.68 (br s, 2H), 3.14 (br s, 2H), 2.17-2.08 (m, 2H), 2.04 (s, 6H), 1.98 (s, 3H), 1.52 (br s, 2H); MS (EI) for C₃₄H₃₈FN₇O: 580 (MH⁺).

### N-Azetidin-3-yl-5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-amine

¹H NMR (400 MHz, CD₃OD) δ 7.57-7.48 (m, 2H), 7.42 (dd, 1H), 7.24 (d, 1H), 7.13-7.06 (m, 2H), 7.01-6.89 (m, 3H), 6.75 (s, 1H), 4.85-4.75 (m, 1H), 4.45 (s, 2H), 4.32-4.24 (m, 2H), 4.07-3.98 (m, 2H), 3.93-3.75 (m, 6H), 2.60 (s, 3H), 2.07 (s, 3H); MS (EI) for C₃₂H₃₂FN₇O: 550 (MH⁺).

### 5-[(4-Fluorophenyl)methyl]-4-methyl-6-[7-(2-methyl-1H-benzimidazol-5-yl)-2,3-dihydro-1,4-benzoxazepin-4(5H)-yl]-N-(1-methylethyl)pyrimidin-2-amine

¹H NMR (400 MHz, DMSO-d₆) δ 7.60-7.45 (m, 2H), 7.42 (dd, 1H), 7.22-7.08 (m, 5H), 7.02-6.90 (m, 2H), 6.37 (d, 1H), 4.43 (br s, 2H), 4.23-4.16 (m, 2H), 3.92-3.78 (m, 3H), 3.70-3.62 (m, 2H), 3.32-3.22 (m, 4H), 1.97 (s, 3H), 0.99 (br s, 6H); MS (EI) for C₃₂H₃₃FN₆O: 537 (MH⁺).

### Example 4

### Methyl (5-{(4-[2-amino-6-methyl-5-(1-methylethyl)pyrimidin-4-yl]-2,3,4,5-tetrahydro-1,4-benzoxazepin-7-yl}-1H-benzimidazol-2-yl)carbamate

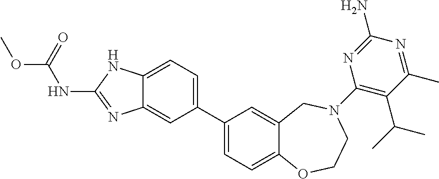

### Step 1:

A mixture of (4-{[(1,1-dimethylethyl)oxy]carbonyl}-2,3,4,5-tetrahydro-1,4-benzoxazepin-7-yl)boronic acid (1.58 g, 5.4 mmol), 4-bromo-2-nitroaniline (1.17 g, 5.4 mmol), cesium carbonate (3.52 mg, 10.8 mmol), 1,1′-bis(diphenylphosphino)ferrocene-palladium(II)dichloride dichloromethane complex (395 mg, 0.54 mmol) in dioxane (20 mL) and water (4 mL) was heated at 90° C. for 3.5 h and was then cooled to rt. The mixture was then diluted with water and ethyl acetate. The mixture was filtered through celite, and then the resulting filtrate was partitioned. The aqueous phase was extracted twice with ethyl acetate. The organic extracts were combined, dried over magnesium sulfate, filtered, and concentrated in vacuo. The residue was purified by gradient silica gel chromatography (80% hexanes:20% ethyl acetate to 50% ethyl acetate:50% hexanes) to provide 1,1-dimethylethyl 7-(4-amino-3-nitrophenyl)-2,3-dihydro-1,4-benzoxazepine-4(5H)-carboxylate (1.59 g, 4.44 mmol, 82% yield) as a bright yellow-orange solid. ¹H NMR (400 MHz, DMSO-d₆) δ 8.16 (d, 1H), 7.73 (d, 1H), 7.52 (s, 2H), 7.49-7.39 (m, 2H), 7.09 (d, 1H), 7.06-6.91 (m, 1H), 4.56-4.37 (m, 2H), 4.11-3.95 (m, 2H), 3.78-3.60 (m, 2H), 1.40-1.22 (m, 9H); MS (EI) for C₂₀H₂₃N₃O₅: 330 (MH⁺-tert-butyl).

### Step 2:

To a solution of 1,1-dimethylethyl 7-(4-amino-3-nitrophenyl)-2,3-dihydro-1,4-benzoxazepine-4(5H)-carboxylate (10 g, 26 mmol) in dioxane (50 mL) was added hydrogen chloride in dioxane (4 M, 50 mL, 200 mmol), and the mixture was stirred at rt for 16 h. The volatile materials were then removed to provide 2-nitro-4-(2,3,4,5-tetrahydro-1,4-benzoxazepin-7-yl)aniline dihydrochloride in quantitative yield. MS (EI) for C₁₅H₁₅N₃O₃: 286 (MH⁺).

### Step 3:

A solution of 2-nitro-4-(2,3,4,5-tetrahydro-1,4-benzoxazepin-7-yl)aniline hydrochloride salt (285 mg, 1 mmol), 4-chloro-6-methyl-5-(1-methylethyl)pyrimidin-2-amine (186 mg, 1 mmol), and diisopropylethylamine (348 uL, 2 mmol) in NMP (2 mL) was heated to 120° C. for 22 h and was then cooled to rt. Water and ethyl acetate was then added and an insoluble orange solid was removed by filtration. The resulting filtrate was partitioned and the aqueous phase extracted with ethyl acetate. The combined organic extracts were dried over sodium sulfate, filtered, and concentrated in vacuo. The residue was purified by silica gel gradient chromatography (100% dichloromethane to 5% methanol:95% dichloromethane) to provide 4-[7-(4-amino-3-nitrophenyl)-2,3-dihydro-1,4-benzoxazepin-4(5H)-yl]-6-methyl-5-(1-methylethyl)pyrimidin-2-amine (161 mg, 0.37 mmol, 37% yield) as yellow/orange oil. ¹H NMR (400 MHz, CDCl₃) δ 8.33 (d, 1H), 7.60 (dd, 1H), 7.43-7.36 (m, 2H), 7.10 (d, 1H), 6.88 (d, 1H), 6.12 (br s, 3H), 4.58 (s, 2H), 4.33 (s, 2H), 4.28-4.23 (m, 2H), 3.69-3.63 (m, 2H), 3.34-3.24 (m, 1H), 2.42 (s, 3H), 1.33 (d, 6H); MS (EI) for C₂₃H₂₆N₆O₃: 435 (MH⁺).

### Step 4:

To a solution of 4-[7-(4-amino-3-nitrophenyl)-2,3-dihydro-1,4-benzoxazepin-4(5H)-yl]-6-methyl-5-(1-methylethyl)pyrimidin-2-amine (161 mg, 0.37 mmol) in methanol (3 mL) was added 10% palladium on carbon (wet, 50 mg). The resulting suspension was subjected to an atmosphere of hydrogen for 1 h. Then an additional 100 mg of 10% palladium on carbon was added, and the suspension was re-subjected to an atmosphere of hydrogen for 2.5 h. The catalyst was then removed by filtration, and the filtrate was concentrated in vacuo to provide 4-{4-[2-amino-6-methyl-5-(1-methylethyl)pyrimidin-4-yl]-2,3,4,5-tetrahydro-1,4-benzoxazepin-7-yl}benzene-1,2-diamine (122.2 mg, 0.302 mmol, 75% yield) as a dark brown film. ¹H NMR (400 MHz, CDCl₃) δ 7.38-7.32 (m, 2H), 7.05 (d, 1H), 6.93-6.88 (m, 2H), 6.79-6.73 (m, 1H), 4.56 (s, 2H), 4.29 (s, 2H), 4.27-4.22 (m, 2H), 3.68-3.62 (m, 2H), 3.49-3.42 (m, 4H), 3.36-3.27 (m, 1H), 2.41 (s, 3H), 1.32 (d, 6H); MS (EI) for C₂₃H₂₈N₆O: 405 (MH⁺).

### Step 5:

A solution of 4-{4-[2-amino-6-methyl-5-(1-methylethyl)pyrimidin-4-yl]-2,3,4,5-tetrahydro-1,4-benzoxazepin-7-yl}benzene-1,2-diamine (122 mg, 0.30 mmol) and 1,3-bis(methoxycarbonyl)-2-methyl-thiopseudourea (62 mg, 0.3 mmol) in acetic acid (1.5 mL) was heated to 80° C. for 20 min and was then cooled to rt. The acetic acid was removed in vacuo. The residue was purified by preparative reverse phase HPLC to provide methyl (5-{4-[2-amino-6-methyl-5-(1-methylethyl)pyrimidin-4-yl]-2,3,4,5-tetrahydro-1,4-benzoxazepin-7-yl}-1H-benzimidazol-2-yl)carbamate (103.4 mg, 0.212 mmol, 71% yield) as a beige solid. ¹H NMR (400 MHz, DMSO-d₆) δ 7.51 (br s, 1H), 7.49-7.42 (m, 2H), 7.39-7.30 (m, 1H), 7.27-7.19 (m, 1H), 7.06 (d, 1H), 6.02 (br s, 2H), 4.28-4.17 (m, 4H), 3.68 (br s, 3H), 3.58-3.50 (m, 2H), 3.29-3.19 (m, 1H), 2.31 (s, 3H), 1.28 (d, 6H); MS (EI) for C₂₆H₂₉N₇O₃: 488 (MH⁺).

### Example 5

### N-({6,6-dimethyl-4-[7-(2-methyl-1H-benzimidazol-5-yl)-2,3-dihydro-1,4-benzoxazepin-4(5H)-yl]-5,6,7,8-tetrahydroquinazolin-2-}methyl)acetamide

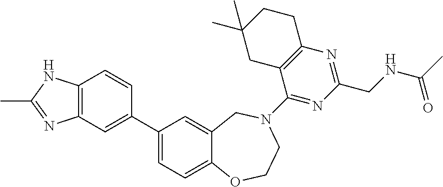 [SMILES: CC(=O)NCc1nc2c(c(N3CCOc4ccc(-c5ccc6[nH]c(C)nc6c5)cc4C3)n1)CC(C)(C)CC2]

### Step 1:

A mixture of 7-bromo-2,3,4,5-tetrahydro-1,4-benzoxazepine hydrochloride (3.06 g, 12.90 mmol), (4-chloro-6,6-dimethyl-5,6,7,8-tetrahydroquinazolin-2-yl)methyl acetate (3.40 g, 11.40 mmol) and N,N-diisopropylethylamine (7.0 mL, 40.0 mmol) in 1-methyl-2-pyrrolidinone (20.0 mL) was stirred at 90° C. for 4 hours. After cooling to room temperature the reaction mixture was partitioned between water and dichloromethane. The organic layer was separated washed with saturated aqueous sodium hydrogencarbonate (25 mL), dried over sodium sulfate, filtered and concentrated to give [4-(7-bromo-2,3-dihydro-1,4-benzoxazepin-4(5H)-yl)-6,6-dimethyl-5,6,7,8-tetrahydroquinazolin-2-yl]methyl acetate that was carried forward without further purification. MS (EI) for C₂₂H₂₆BrN₃O₃: 461 (MH⁺).

### Step 2:

To a solution of [4-(7-bromo-2,3-dihydro-1,4-benzoxazepin-4(5H)-yl)-6,6-dimethyl-5,6,7,8-tetrahydroquinazolin-2-yl]methyl acetate as obtained in step 1 in a mixture of methanol (50 mL) and water (10 mL) was added lithium hydroxide (0.40 g, 13.40 mmol) and the reaction mixture was stirred 30 minutes at room temperature. It was concentrated and partitioned between saturated aqueous sodium hydrogencarbonate and ethyl acetate. The organic layer was separated washed with brine, dried over sodium sulfate, filtered and concentrated. Gradient silica gel chromatography (hexane:ethyl acetate 4:1 to 1:1) provided [4-(7-bromo-2,3-dihydro-1,4-benzoxazepin-4(5H)-yl)-6,6-dimethyl-5,6,7,8-tetrahydroquinazolin-2-yl]methanol (3.7 g, 77%). ¹H NMR (400 MHz, CHCl₃-d₃): 7.37 (d, 1H), 7.29 (dd, 1H), 6.89 (d, 1H), 4.56 (s, 2H), 4.52 (s, 2H), 4.23 (t, 2H), 3.91 (t, 2H), 3.70 (t, 1H), 2.81 (t, 2H), 2.38 (s, 2H), 1.65 (t, 2H), 0.96 (s, 6H); MS (EI) for C₂₀H₂₄BrN₃O₂: 419 (MH⁺).

### Step 3:

A combined solution of [4-(7-bromo-2,3-dihydro-1,4-benzoxazepin-4(5H)-yl)-6,6-dimethyl-5,6,7,8-tetrahydroquinazolin-2-yl]methanol (2.20 g, 5.30 mmol) and phosphorus oxychloride (5.0 mL, 53.65 mmol) in chloroform (5.0 mL) was heated to 75° C. for 150 minutes. After cooling it to room temperature, the reaction mixture was concentrated and the resulting residue was partitioned between saturated aqueous sodium hydrogencarbonate and dichloromethane. The organic layer was separated washed with 1M aqueous sodium hydroxide dried over sodium sulfate, filtered and concentrated. Gradient silica gel chromatography (hexane:ethyl acetate 9:1 to 1:1) provided 7-bromo-4-[2-(chloromethyl)-6,6-dimethyl-5,6,7,8-tetrahydroquinazolin-4-yl]-2,3,4,5-tetrahydro-1,4-benzoxazepine (1.9 g, 83%). MS (EI) for C₂₀H₂₃BrClN₃O: 437 (MH⁺).

### Step 4:

To a combined solution of acetamide (37 mg, 0.63 mmol) and lithium hexamethyldisilazide (1 M in hexane, 1.50 mL, 1.50 mmol) in N,N-dimethylformamide (4.0 mL) was added a solution of 7-bromo-4-[2-(chloromethyl)-6,6-dimethyl-5,6,7,8-tetrahydroquinazolin-4-yl]-2,3,4,5-tetrahydro-1,4-benzoxazepine (91 mg, 0.21 mmol) in N,N-dimethylformamide (2.0 mL) and the reaction mixture was stirred at 100° C. for 16 hours. After cooling to room temperature the reaction mixture was partitioned between saturated aqueous sodium hydrogencarbonate and dichloromethane. The organic layer was separated and the aqueous layer was extracted with dichloromethane (2×). The combined organic layers were washed with water, dried over sodium sulfate, filtered and concentrated to give N-{[4-(7-bromo-2,3-dihydro-1,4-benzoxazepin-4(5H)-yl)-6,6-dimethyl-5,6,7,8-tetrahydroquinazolin-2-yl]methyl}acetamide (50 mg, 52%) and used without further purification. MS (EI) for C₂₂H₂₇BrN₄O₂: 460 (MH⁺).

### Step 5:

A mixture of N-{[4-(7-bromo-2,3-dihydro-1,4-benzoxazepin-4(5H)-yl)-6,6-dimethyl-5,6,7,8-tetrahydroquinazolin-2-yl]methyl}acetamide (50 mg, 0.11 mmol) 1,1-dimethylethyl 2-methyl-6-(4,4,5,5-tetramethyl-1,3,2-dioxaborolan-2-yl)-1H-benzimidazole-1-carboxylate (42 mg, 0.16 mmol), bis(diphenylphosphino)ferrocene]dichloropalladium(II) (7 mg, 0.011 mmol), cesium carbonate (0.18 g, 0.55 mmol) in a mixture of 1,4-dioxane (1.8 mL), and water (0.2 mL) was reacted in a microwave apparatus at 110° C. for 30 minutes The reaction mixture was cooled to room temperature and partitioned between saturated aqueous sodium hydrogencarbonate and dichloromethane. The organic layer was separated washed with water dried over sodium sulfate, filtered and concentrated. To a solution of the resulting residue in methanol a solution of 4M anhydrous hydrogen chloride in 1,4-dioxane (2.0 mL) was added and the reaction mixture was heated to reflux for 30 minutes. After cooling it to room temperature the reaction mixture was concentrated, dissolved in methanol (2 mL) and purified by preparative reverse phase HPLC (0.1% aqueous ammonium acetate buffered aqueous acetonitrile mobile phase) to give N-({6,6-dimethyl-4-[7-(2-methyl-1H-benzimidazol-5-yl)-2,3-dihydro-1,4-benzoxazepin-4(5H)-yl]-5,6,7,8-tetrahydroquinazolin-2-yl}methyl)acetamide (8 mg, 14%). ¹H NMR (400 MHz, d₆-DMSO) δ 8.05 (t, 1H), 7.64 (d, 1H), 7.63 (s, 1H), 7.48 (dd, 1H), 7.37 (dd, 1H), 7.03 (d, 1H), 4.66 (s, 2H), 4.31 (m, 2H), 4.17 (d, 2H), 3.88 (m, 2H), 2.70 (t, 2H), 2.47 (s, 3H), 1.84 (s, 2H), 1.84 (s, 3H), 1.60 (t, 2H), 0.87 (s, 6H); MS (ES) for C₃₀H₃₄N₆O₂: 511 (MH⁺).

Proceeding according to the method of example 5 and reaction of [4-(7-bromo-2,3-dihydro-1,4-benzoxazepin-4(5H)-yl)-6,6-dimethyl-5,6,7,8-tetrahydroquinazolin-2-yl]methanol with DAST in step 3 then proceeding to step 5 the following compounds of the invention were prepared:

### 4-[2-(Fluoromethyl)-6,6-dimethyl-5,6,7,8-tetrahydroquinazolin-4-yl]-7-(2-methyl-1H-benzimidazol-5-yl)-2,3,4,5-tetrahydro-1,4-benzoxazepine

¹H NMR (400 MHz, d₆-DMSO) δ 7.69 (s, 1H), 7.67 (d, 1H), 7.54 (d, 1H), 7.50 (dd, 1H), 7.44 (dd, 1H), 7.03 (d, 1H), 5.23 (d, 2H), 4.66 (s, 2H), 4.30 (m, 2H), 3.89 (s, 1H), 2.73 (t, 2H), 2.54 (s, 3H), 1.91 (s, 2H), 1.61 (t, 2H), 0.86 (s, 6H); MS (ES) for C₂₈H₃₀FN₅O: 472 (MH⁺).

### Example 6

### 6-{4-[2-Amino-6-methyl-5-(1-methylethyl)pyrimidin-4-yl]-2,3,4,5-tetrahydro-1,4-benzoxazepin-7-yl}-3H-imidazo[4,5-b]pyridin-2-amine

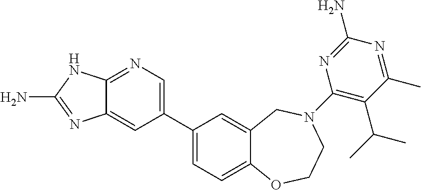 [SMILES: Cc1nc(N)nc(N2CCOc3ccc(-c4cnc5[nH]c(N)nc5c4)cc3C2)c1C(C)C]

### Step 1:

A suspension of 5-bromo-3-nitropyridin-2-amine (4.84 g, 22.2 mmol), (4-{[(1,1-dimethylethyl)oxy]carbonyl}-2,3,4,5-tetrahydro-1,4-benzoxazepin-7-yl)boronic acid (6.51 g, 22.2 mmol), dichloro[1,1-bis(diphenyl)phosphino]ferrocenepalladium (II) dichloromethane adduct (1.60 g, 10 mol %) in dioxane (75 mL) and water (15 mL) was degassed with nitrogen, and then cesium carbonate (14.46 g, 44.4 mmol) was added. The reaction mixture was stirred at 90° C. overnight. The mixture was cooled to room temperature, water (150 ml) was added and stirred for 30 min to give a precipitate. The product 1,1-dimethylethyl-7-(6-amino-5-nitropyridin-3-yl)-2,3-dihydro-1,4-benzoxazepine-4(5H)-carboxylate (8.1 g, 94% yield) was collected by filtration, dried under vacuum. MS (EI) for C₁₉H₂₂N₄O₅: 387.1 (MH⁺).

### Step 2:

A suspension of 1,1-dimethylethyl-7-(6-amino-5-nitropyridin-3-yl)-2,3-dihydro-1,4-benzoxazepine-4(5H)-carboxylate (6.12 g, 15.84 mmol), palladium on carbon (0.6 g) in acetic acid (100 mL) was degassed with nitrogen for 10 minutes. The reaction mixture was hydrogenated (45 psi) on a parr shaker for 60 minutes. Upon completion of hydrogenation, the reaction mixture was filtered through a pad of Celite®. The filtrate was concentrated and the residue was diluted with ethyl acetate (200 ml), washed with water, saturated sodium bicarbonate solution and brine then dried over sodium sulfate and filtered. Evaporation of ethyl acetate afforded 1,1-dimethylethyl-7-(5,6-diaminopyridin-3-yl)-2,3-dihydro-1,4-benzoxazepine-4(5H)-carboxylate (5.6 g, 99% yield). ¹H NMR (400 MHz, CD₃OD): 7.56-7.27 (m, 4H), 7.08-7.02 (m, 1H), 4.51 (s, 2H), 4.07-4.03 (m, 2H), 3.86-3.74 (m, 2H), 1.3 (s, 9H).

### Step 3:

A solution of 1,1-dimethylethyl-7-(5,6-diaminopyridin-3-yl)-2,3-dihydro-1,4-benzoxazepine-4(5H)-carboxylate (5.64 g, 15.84 mmol) and 1,3-bis(benzyloxycarbonyl)-2-methyl-2-thiopseudourea (11.8 g, 32.92 mmol) in acetic acid (30 mL) was heated to 86° C. for 3 h. After cooling to room temperature, ethyl acetate (100 mL) was added and the precipitate was collected by filtration, washed several times with ethyl acetate, and dried to give 1,1-dimethylethyl-7-[2-({[(phenylmethyl)oxy]carbonyl}amino)-1H-imidazo[4,5-b]pyridine-6-yl]-2,3-dihydro-1,4-benzoxazepine-4(5H)-carboxylate (4.50 g, 55% yield). ¹HNMR (400 MHz, DMSO-d₆): 11.9 (s, br, 2H), 8.42 (s, 1H), 7.90 (s, 1H), 7.52-7.36 (m, 7H), 5.28 (s, 2H), 4.60-4.34 (m, 2H), 4.18-4.02 (m, 2H), 3.79-3.67 (m, 2H), 1.38 (s, 9H).

### Step 4:

To the solution of 1,1-dimethylethyl-7-[2-({[(phenylmethyl)oxy]carbonyl}amino)-1H-imidazo[4,5-b]pyridine-6-yl]-2,3-dihydro-1,4-benzoxazepine-4(5H)-carboxylate (4.5 g, 8.73 mmol) in methanol (20 ml) was added 4 N HCl in dioxane at room temperature. The reaction mixture was then heated to 55° C. for 3 hours. After cooling to room temperature, the precipitate was collected by filtration, and washed with a minimum of methanol and dried to give phenylmethyl[6-(2,3,4,5-tetrahydro-1,4-benzoxazepin-7-yl)-1H-imidazo[4,5-b]pyridin-2-yl]carbamate hydrochloride (3.0 g, 76% yield). ¹HNMR (400 MHz, CD₃OD): 8.59 (m, 1H), 8.51 (s, 1H), 7.85 (m, 1H), 7.79-7.75 (m, 1H), 5.37 (s, 2H), 4.53 (s, 2H), 4.37-4.32 (m, 2H), 3.67-3.64 (m, 2H).

### Step 5:

To a stirred solution of phenylmethyl[6-(2,3,4,5-tetrahydro-1,4-benzoxazepin-7-yl)-1H-imidazo[4,5-b]pyridin-2-yl]carbamate hydrochloride (265 mg, 0.58 mmol) and 4-chloro-5-isopropyl-6-methylpyrimidin-2-amine (108 mg, 0.58 mmol) in NMP (2 mL) was added diisopropylethylamine (0.42 mL, 2.34 mmol) at room temperature. The reaction mixture was heated to 120° C. and stirred overnight. The reaction was cooled to room temperature and diluted with water, then extracted with ethyl acetate (20 mL×3). The extracts were dried over magnesium sulfate, filtered and concentrated under reduced pressure. The resulting residue was purified by preparative reverse phase HPLC using an ammonium acetate buffered aqueous acetonitrile gradient mobile phase and the combined pure fractions lyophilized to afford 6-{4-[2-amino-6-methyl-5-(1-methylethyl)pyrimidin-4-yl]-2,3,4,5-tetrahydro-1,4-benzoxazepin-7-yl}-3H-imidazo[4,5-b]pyridin-2-amine. ¹H NMR (400 MHz, DMSO-d₆): δ 8.12 (s, 1H), 7.50 (d, 3H), 7.31 (s, 1H), 7.07 (d, 1H), 6.67 (s, 2H), 6.02 (s, 2H), 4.25 (s, 4H), 3.51 (d, 2H), 3.31 (m, 1H), 2.30 (s, 3H), 1.26 (d, 6H); MS (EI) for C₂₃H₂₆N₈O: 431.3 (MH⁺).

Proceeding as in Example 1 and replacing 1,3-bis(benzyloxycarbonyl)-2-methyl-2-thiopseudourea with triethyl orthoformate in step 3 and 4-chloro-5-isopropyl-6-methylpyrimidin-2-amine with alternative starting reagents in step 5, the following compounds of the invention were prepared.

### 5-[(4-Fluorophenyl)methyl]-4-[7-(3H-imidazo[4,5-b]pyridin-6-yl)-2,3-dihydro-1,4-benzoxazepin-4(5H)-yl]-6-methylpyrimidin-2-amine

¹H NMR (400 MHz, DMSO-d6): δ 8.47 (dd, 2H), 8.12 (s, 1H), 7.95 (s, 1H), 7.53 (s, 1H), 7.12 (t, 2H), 7.05 (t, 3H), 6.88 (d, 1H), 6.13 (s, 2H), 4.34 (s, 2H), 4.22 (s, 2H), 3.83 (s, 2H), 3.65 (s, 2H), 1.97 (s, 3H); MS (EI) for C27H24FN7O: 482.2 (MH+).

### 1-{4-[7-(3H-Imidazo[4,5-b]pyridin-6-yl)-2,3-dihydro-1,4-benzoxazepin-4(5H)-yl]-7-(methyloxy)quinazolin-2-yl}-N,N-dimethylmethanamine

1H NMR (400 MHz, DMSO-d6): δ 8.62 (t, 1H), 8.45 (d, 1H), 8.17 (d, 1H), 7.90 (t, 1H), 7.74 (t, 1H), 7.52 (dt, 1H), 7.12 (t, 1H), 7.05 (dd, 1H), 6.98 (d, 1H), 5.04 (s, 2H), 4.44 (d, 2H), 4.18 (s, 2H), 3.86 (s, 3H), 3.41 (s, 2H), 2.08 (d, 6H); MS (EI) for C27H27N70O2: 482.3 (MH+).

### 4-[7-(3H-Imidazo[4,5-b]pyridin-6-yl)-2,3-dihydro-1,4-benzoxazepin-4(5H)-yl]-6-methyl-5-(1-methylethyl)pyrimidin-2-amine

1H NMR (400 MHz, DMSO-d6): δ 8.60 (s, 1H), 8.45 (s, 1H), 8.17 (s, 1H), 7.62-7.52 (m, 2H), 7.09 (d, 1H), 6.00 (s, 2H), 4.26 (d, 4H), 3.52 (s, 2H), 3.18 (dt, 1H), 2.28 (s, 3H), 1.22 (t, 6H); MS (EI) for C23H25N7O: 416.0 (MH+).

### 4-[7-(3H-Imidazo[4,5-b]pyridin-6-yl)-2,3-dihydro-1,4-benzoxazepin-4(5H)-yl]-6,6-dimethyl-5,6,7,8-tetrahydroquinazolin-2-amine

¹H-NMR (400 MHz, d4-MeOH): 8.65 (br s, 1H), 8.43 (s, 1H), 8.21 (br s, 1H), 7.63 (d, 1H), 7.53 (dd, 1H), 7.11 (dd, 1H), 4.73 (s, 2H), 4.35 (tr, 2H), 3.95 (tr, 2H), 2.62 (tr, 2H), 2.39 (s, 2H), 1.61 (tr, 2H), 0.90 (s, 6H). MS EI for C25H27N7O: 443 (MH+).

### 5-[(4-Fluorophenyl)methyl]-4-methyl-6-[7-(2-methyl-3H-imidazo[4,5-b]pyridin-6-yl)-2,3-dihydro-1,4-benzoxazepin-4(5H)-yl]pyrimidin-2-amine

¹H NMR (400 MHz, DMSO-d₆): δ 8.29 (s, 1H), 7.94 (s, 1H), 7.50 (d, 1H), 7.11 (t, 2H), 7.05 (t, 4H), 6.86 (s, 1H), 6.12 (s, 2H), 4.33 (s, 2H), 4.22 (s, 2H), 3.83 (s, 2H), 3.65 (s, 2H), 2.54 (s, 3H), 1.97 (s, 3H); MS (EI) for C₂₈H₂₆FN₇O: 496.2 (MH⁺).

### 4-Methyl-5-(1-methylethyl)-6-[7-(2-methyl-3H-imidazo[4,5-b]pyridin-6-yl)-2,3-dihydro-1,4-benzoxazepin-4(5H)-yl]pyrimidin-2-amine

¹H NMR (400 MHz, DMSO-d₆): δ 8.47 (d, 1H), 7.98 (d, 1H), 7.65-7.48 (m, 2H), 7.08 (t, 1H), 6.03 (s, 2H), 4.27 (d, 4H), 3.54 (s, 2H), 3.21 (m, 1H), 2.54 (s, 3H), 2.30 (s, 3H), 1.25 (d, 6H); MS (EI) for C₂₄H₂₇N₇O: 430.3 (MH⁺).

### 6,6-Dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-amine

¹H NMR (400 MHz, DMSO-d₆): 8.51 (s, 1H), 8.03 (s, 1H), 7.66 (d, 1H), 7.53 (dd, 1H), 7.17 (d, 1H), 5.87 (s, 2H), 4.50 (s, 2H), 4.29 (m, 2H), 3.73 (m, 2H), 2.55 (s, 3H), 2.33 (s, 2H), 1.86 (s, 2H), 1.52 (t, 2H), 0.92 (s, 6H); MS (EI) for C₂₆H₂₉N₇O: 456 (MH⁺).

### 4-[9-Fluoro-7-(2-methyl-1H-benzimidazol-6-yl)-2,3-dihydro-1,4-benzoxazepin-4(5H)-yl]-6-methyl-5-(1-methylethyl)pyrimidin-2-amine

The title compound was prepared using 4-chloro-5-isopropyl-6-methylpyrimidin-2-amine and 7-bromo-9-fluoro-2,3,4,5-tetrahydro-1,4-benzoxazepine hydrochloride.

¹H NMR (400 MHz, d₆-DMSO) δ 7.67 (s, 1H), 7.51 (d, 1H), 7.48 (d, 1H), 7.39 (dd, 1H), 7.34 (s, 1H), 6.04 (s, 2H), 4.31 (m, 4H), 3.57 (m, 2H), 3.20 (m, 1H), 2.49 (s, 3H), 2.30 (s, 3H), 1.26 (d, 6H); MS (ES) for C₂₅H₂₇FN₆O: 447 (MH⁺).

### BIOLOGICAL EXAMPLES

Compounds of the Invention have activity for PI3K-alpha, mTOR, or for both. Compounds of this invention have been tested using the assays described in Biological Examples 1 and 3 and have been determined to be inhibitors of PI3K-alpha, mTOR, or for both.

Suitable in vitro assays for measuring PI3K, mTORc1, and mTORc2 activity and the inhibition thereof by compounds are known in the art. Biological Examples, Example 1, 2, and 3 describe in vitro assay for measuring PI3K and mTOR activity. Cell-based assays for measurement of in vitro efficacy in treatment of cancer are known in the art. Biological Examples, Example 5 and 6 describe assays to measure in vitro cell activity. Suitable in vivo models for cancer are known to those of ordinary skill in the art. Biological Examples 7, 8, 9, 10, 11, 12, and 13 describe in vivo models for prostate adenocarcinoma, glioblastoma, lung carcinoma, and melanoma. Following the examples disclosed herein, as well as that disclosed in the art, a person of ordinary skill in the art can determine the PI3K-inhibitory and/or mTOR-inhibitory activity of a Compound of this invention.

Thus, compounds of Formula I are useful for treating diseases, particularly cancer in which activity against PI3K-alpha, mTOR, or both contributes to the pathology and/or symptomatology of the disease. For example, cancer in which activity against PI3K-alpha, mTOR, or both contributes to its pathology and/or symptomatology include breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

### Biological Example 1

### mTOR/GbL/Raptor (mTORC1) ELISA Assay

The measurement of mTORC1 enzyme activity was performed in an ELISA assay format following the phosphorylation of 4E-BP1 protein. All experiments were performed in the 384-well format. Generally, 0.5 μL DMSO containing varying concentrations of the test compound was mixed with 15 μL enzyme solution. Kinase reactions were initiated with the addition of 15 μL of substrates-containing solution. The assay conditions were as follows; 0.2 nM mTORC1, 10 μM ATP and 50 nM NHis-tagged 4E-BP1 in 20 mM Hepes, pH 7.2, 1 mM DTT, 50 mM NaCl, 10 mM MnCl₂, 0.02 mg/mL BSA, 0.01% CHAPS, 50 mM β-glycerophosphate. Following an incubation of 120 minutes at ambient temperature, 20 μL of the reaction volume was transferred to a Ni-Chelate-coated 384-well plate. The binding step of the 4E-BP1 protein proceeded for 60 minutes, followed by washing 4 times each with 50 μL of Tris-buffered saline solution (TBS). Anti-phospho-4E-BP1 rabbit-IgG (20 μL, 1:5000) in 5% BSA-TBST (0.2% Tween-20 in TBS) was added and further incubated for 60 minutes. Incubation with a secondary HRP-tagged anti-IgG was similarly performed after washing off the primary antibody (4 washes of 50 μL). Following the final wash step with TBST, 20 μL of SuperSignal ELISA Femto (Pierce Biotechnology) was added and the luminescence measured using an EnVision plate reader.

Using this assay, compounds of the invention, preferably have an mTOR-inhibitory activity of between about 0.0001 nM and 500 nM (IC₅₀). In another embodiment the Compound of the Invention has an mTOR-inhibitory activity of about of between about 0.001 nM and 250 nM. In another embodiment the Compound of the Invention has an mTOR-inhibitory activity of between about 0.001 nM and 250 nM. In another embodiment the Compound of the Invention has an mTOR-inhibitory activity of 0.01 nM and 100 nM. In another embodiment the Compound of the Invention has an mTOR-inhibitory activity of 0.1 nM and 50 nM.

As numbered in Table 1, Compounds 4-5, 11-17, 24-29, 33-36, 39-42, 44-48 and 50-52 have an IC₅₀ in this assay of less than or equal to 100 nM.

As numbered in Table 1, Compounds 3, 7-8, 10, 18-21, and 23 have an IC₅₀ in this assay of greater than 100 nM but less than or equal to 500 nM.

As numbered in Table 1, Compounds 1-2, 6, 9, 22, 30-32, 37-38, 43 and 49 have an IC₅₀ in this assay of greater than 500 nM but less than or equal to 2500 nM.

### Biological Example 2

### Immune-Complex mTORC2 Kinase (mTORC2 IP-Kinase) Assay

HeLa (ATCC) cells are grown in suspension culture and lysed in ice-cold lysis buffer containing 40 mM HEPES pH 7.5, 120 mM NaCl, 1 mM EDTA, 10 mM sodium pyrophosphate, 10 mM β-glycerophosphate, 10 mM NaF, 10 mM NaN₃, one tablet of protease inhibitors (Complete-Mini, EDTA-free, Roche), 0.3% cholamidopropyldimethylammoniopropane sulfonate (CHAPS), 1 mM AEBSF, 0.5 mM benzamidine HCl, 20 μg/mL heparin, and 1.5 mM Na₃VO₄. The mTORC2 complex is immunoprecipitated with anti-RICTOR antibody for 2 h. The immune complexes are immobilized on Protein A sepharose (GE Healthcare, 17-5280-01), washed sequentially 3 times with wash buffer (40 mM HEPES pH 7.5, 120 mM NaCl, 10 mM β-glycerophosphate, 0.3% CHAPS, 1 mM AEBSF, 20 μg/mL heparin, 1.5 mM Na₃VO₄, and Complete-Mini, EDTA-free) and resuspended in kinase buffer (40 mM HEPES, pH 7.5, 120 mM NaCl, 0.3% CHAPS, 20 μg/mL heparin, 4 mM MgCl₂, 4 mM MnCl₂, 10% Glycerol, and 10 mM DTT). The immune complexes (equivalent to 1×10⁷ cells) are pre-incubated at 37° C. with a test compound or 0.6% DMSO for 5 min, and then subjected to a kinase reaction for 8 min in a final volume of 33 μL (including 5 μL bed volume) containing kinase buffer, 50 μM ATP, and 0.75 μg full length dephosphorylated AKT1. Kinase reactions are terminated by addition of 11 μL 4×SDS sample buffer containing 20% β-mercaptoethanol and resolved in a 10% Tris Glycine gels. The gels are transferred onto PVDF membrane at 50 V for 20 h at 4 C. The membranes are blocked in 5% non-fat milk in TBST for 1 h and incubated overnight at 4° C. with 1/1000 dilution of rabbit anti-pAKT (S473) (Cell Signaling Technology, 4060) in 3% BSA/TBST. The membranes are washed 3 times in TBST and incubated for 1 h with a 1/10000 dilution of secondary goat anti-rabbit HRP antibody (Cell Signaling Technology, 2125) in 5% non-fat milk/TBST. The signal is detected using Amersham ECL-plus. The scanned data are analyzed using ImageQuant software. IC₅₀ for the test compound is determined relative to DMSO treated sample using XLfit4 software.

### Biological Example 3

### pS6 (S240/244) ELISA Assay

MCF-7 cells (ATCC) cells were seeded at 24000 cells per well in 96-well plates (Corning, 3904) in DMEM (Cellgro) containing 10% FBS (Cellgro), 1% NEAA (Cellgro) and 1% penicillin-streptomycin (Cellgro). Cells were incubated at 37° C., 5% CO2 for 48 h, and the growth medium was replaced with serum-free DMEM or in medium containing 0.4% BSA. Serial dilutions of the test compound in 0.3% DMSO (vehicle) were added to the cells and incubated for 3 h. To fix the cells, medium was removed and 100A/well of 4% formaldehyde (Sigma Aldrich, F8775) in TBS (20 mM Tris, 500 mM NaCl) was added to each well at RT for 30 min. Cells were washed 4 times with 200 μL TBS containing 0.1% Triton X-100 (Sigma, catalog #T9284). Plates were blocked with 100 μL Odyssey blocking buffer (Li-Cor Biosciences, 927-40000) for 1 h at RT. Anti-pS6 (S240/244) antibody (Cell Signaling Technology, 2215) and anti-total-S6 antibody (R&D systems, MAB5436) were diluted 1:400 in Odyssey blocking buffer, and 50 μL of the antibody solution containing both antibodies was added to one plate to detect pS6 and total S6. After incubation overnight at 4° C., plates were washed 4 times with 200 μL TBS containing 0.1% Tween20 (Bio-Rad, catalog #170-6351) (TBST). Goat anti-rabbit and Goat anti-mouse secondary antibody (Li-Cor Biosciences, catalog #926-32221 and 926-32210) conjugated to IRDye were diluted 1:400 in Odyssey blocking buffer containing 0.1% Tween20. 50 μL of antibody solution containing both antibodies was added to each well and incubated for 1 h at RT. Plates were washed 3 times with 200 μL TBST and 2 times with 200 μL TBS. Fluorescence was read on an Odyssey plate reader. IC50 values were determined based on the ratio of pS6 to total S6 signal for compound treated wells, normalized to the DMSO-treated control wells.

In one embodiment, the Compound of the Invention tested in this assay in MCF-7 cells has an inhibitory activity of 1.5 μM or less. In another embodiment, the Compound of the Invention tested in this assay in MCF-7 cells has an inhibitory activity of 1.0 μM or less.

In another embodiment, the Compound of the Invention tested in this assay in MCF-7 cells has an inhibitory activity of 0.5 μM or less. In one embodiment, the Compound of the Invention tested in this assay in MCF-7 cells has an inhibitory activity of 0.25 μM or less. In one embodiment, the Compound of the Invention tested in this assay in MCF-7 cells has an inhibitory activity of 0.2 μM or less. In one embodiment, the Compound of the Invention tested in this assay in MCF-7 cells has an inhibitory activity of 0.1 μM or less.

### Biological Example 4

### PI3K Biochemical Assays

PI3Kα activity was measured as the percent of ATP consumed following the kinase reaction using luciferase-luciferin-coupled chemiluminescence. Reactions were conducted in 384-well white, medium binding microtiter plates (Greiner). Kinase reactions were initiated by combining test compounds, ATP, substrate (PIP2), and kinase in a 20 μL volume in a buffer solution. The standard PI3Kalpha assay buffer was composed 50 mM Tris, pH 7.5, 1 mM EGTA, 10 mM MgCl₂, 1 mM DTT and 0.03% CHAPS. The standard assay concentrations for enzyme, ATP, and substrate were 3 nM, 1 μM, and 10 μM, respectively. The reaction mixture was incubated at ambient temperature for approximately 2 h. Following the kinase reaction, a 10 μL aliquot of luciferase-luciferin mix (Promega Kinase-Glo) was added and the chemiluminescence signal measured using a Victor2 or EnVision (Perkin Elmer). Total ATP consumption was limited to 40-60% and IC50 values of control compounds correlate well with literature references. Substituting PI3Kα with PI3Kβ, PI3Kγ, or PINKδ, the inhibitory activity of the compounds for the other isoforms of PI3K were measured. For the PI3Kβ and PI3Kδ assays, enzyme concentrations were 10 nM and 4 nM, respectively. For the PI3Kγ assay, enzyme concentration was 40 nM, the incubation time was 1 h, and the concentration of MgCl₂ in the assay buffer was 5 mM.

As numbered in Table 1, Compounds 1-11, 14-15, 17, 20, 22, 24-30, 32-42 and 44-52 have an IC₅₀ in the PI3K-alpha assay of less than or equal to 100 nM.

As numbered in Table 1, Compounds 12-13, 16, 18, 21, 23, and 31 have an IC₅₀ in the PI3K-alpha assay of greater than 100 nM but less than or equal to 500 nM.

As numbered in Table 1, Compounds 19 and 43 have an IC₅₀ in the PI3K-alpha assay of greater than 500 nM but less than or equal to 2500 nM.

### Alternative Embodiment

In one embodiment the invention comprises a compound of the invention having a PI3K-alpha-inhibitory activity of about 0.5 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment, the invention comprises a compound of the invention having a PI3K-alpha-inhibitory activity of about 0.35 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment, the invention comprises a compound of the invention having a PI3K-alpha-inhibitory activity of about 0.25 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the compounds of the invention have an PI3K-alpha-inhibitory activity of about 0.1 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the invention comprises a compound of the invention having an PI3K-alpha-inhibitory activity of about 0.05 μM or less and is selective for PI3K-alpha over mTOR by about 5-fold or greater, about 7-fold or greater, or about 10-fold or greater.

### Alternative Embodiment

In one embodiment the invention comprises a compound of the invention having a PI3K-alpha-inhibitory activity of about 2.0 μM or less and an mTOR-inhibitory activity of about 2.0 μM or less and the selectivity for one of the targets over the other does not exceed 3-fold. In another embodiment the invention comprises a compound of the invention having a PI3K-alpha-inhibitory activity of about 1.0 μM or less and an mTOR-inhibitory activity of about 1.0 μM or less and the selectivity for one of the targets over the other does not exceed 3-fold. In another embodiment the invention comprises a compound of the invention having a PI3K-alpha-inhibitory activity of about 0.5 μM or less and an mTOR-inhibitory activity of about 0.5 μM or less and the selectivity for one of the targets over the other does not exceed 3-fold. In another embodiment the invention comprises a compound of the invention having a PI3K-alpha-inhibitory activity of about 0.3 μM or less and an mTOR-inhibitory activity of about 0.3 μM or less and the selectivity for one of the targets over the other does not exceed 3-fold. In another embodiment the invention comprises a compound of the invention having a PI3K-alpha-inhibitory activity of about 0.2 μM or less and an mTOR-inhibitory activity of about 0.2 μM or less and the selectivity for one of the targets over the other does not exceed 2-fold. In another embodiment the invention comprises a compound of the invention having a PI3K-alpha-inhibitory activity of about 0.15 μM or less and an mTOR-inhibitory activity of about 0.15 μM or less and the selectivity for one of the targets over the other does not exceed 2-fold. In another embodiment the invention comprises a compound of the invention having a PI3K-alpha-inhibitory activity of about 0.1 μM or less and an mTOR-inhibitory activity of about 0.1 μM or less. In another embodiment the invention comprises a compound of the invention having a PI3K-alpha-inhibitory activity of about 0.05 μM or less and an mTOR-inhibitory activity of about 0.05 μM or less. In another embodiment the invention comprises a compound of the invention have a PI3K-alpha-inhibitory activity of about 0.02 μM or less and an mTOR-inhibitory activity of about 0.02 or less. In another embodiment the invention comprises a compound of the invention have a PI3K-alpha-inhibitory activity of about 0.01 μM or less and an mTOR-inhibitory activity of about 0.01 μM or less.

### Biological Example 5-11

### Pharmacodynamic Xenograft Tumor Models

Female and male athymic nude mice (NCr) 5-8 weeks of age and weighing approximately 20-25 g are used in the following models. Prior to initiation of a study, the animals are allowed to acclimate for a minimum of 48 h. During these studies, animals are provided food and water ad libitum and housed in a room conditioned at 70-75° F. and 60% relative humidity. A 12 h light and 12 h dark cycle is maintained with automatic timers. All animals are examined daily for compound-induced or tumor-related deaths.

### MCF-7 Breast Adenocarcinoma Model

MCF7 human mammary adenocarcinoma cells are cultured in vitro in DMEM (Cellgro) supplemented with 10% Fetal Bovine Serum (Cellgro), Penicillin-Streptomycin and non-essential amino acids at 37° C. in a humidified 5% CO₂ atmosphere. On day 0, cells are harvested by trypsinization, and 5×10⁶ cells in 100 μL of a solution made of 50% cold Hanks balanced salt solution with 50% growth factor reduced matrigel (Becton Dickinson) implanted subcutaneously into the hindflank of female nude mice. A transponder is implanted into each mouse for identification and data tracking, and animals are monitored daily for clinical symptoms and survival.

Tumors are established in female athymic nude mice and staged when the average tumor weight reached 100-200 mg. A Compound of the Invention is orally administered as a solution/fine suspension in water (with 1:1 molar ratio of 1 NHCL) once-daily (qd) or twice-daily (bid) at 10, 25, 50 and 100 mg/kg for 14 days. During the dosing period of 14-19 days, tumor weights are determined twice-weekly and body weights are recorded daily.

### Colo-205 Colon Model

Colo-205 human colorectal carcinoma cells are cultured in vitro in DMEM (Mediatech) supplemented with 10% Fetal Bovine Serum (Hyclone), Penicillin-Streptomycin and non-essential amino acids at 37° C. in a humidified, 5% CO₂ atmosphere. On day 0, cells are harvested by trypsinization, and 3×10⁶ cells (passage 10-15, >95% viability) in 0.1 mL ice-cold Hank's balanced salt solution are implanted intradermally in the hind-flank of 5-8 week old female athymic nude mice. A transponder is implanted in each mouse for identification, and animals are monitored daily for clinical symptoms and survival.

Tumors are established in female athymic nude mice and staged when the average tumor weight reached 100-200 mg. A Compound of the Invention is orally administered as a solution/fine suspension in water (with 1:1 molar ratio of 1 NHCL) once-daily (qd) or twice-daily (bid) at 10, 25, 50 and 100 mg/kg for 14 days. During the dosing period of 14 days, tumor weights are determined twice-weekly and body weights are recorded daily.

### PC-3 Prostate Adenocarcinoma Model

PC-3 human prostate adenocarcinoma cells are cultured in vitro in DMEM (Mediatech) supplemented with 20% Fetal Bovine Serum (Hyclone), Penicillin-Streptomycin and non-essential amino acids at 37° C. in a humidified 5% CO₂ atmosphere. On day 0, cells are harvested by trypsinization and 3×10⁶ cells (passage 10-14, >95% viability) in 0.1 mL of ice-cold Hank's balanced salt solution are implanted subcutaneously into the hindflank of 5-8 week old male nude mice. A transponder is implanted in each mouse for identification, and animals are monitored daily for clinical symptoms and survival.

Tumors are established in male athymic nude mice and staged when the average tumor weight reached 100-200 mg. A Compound of the Invention is orally administered as a solution/fine suspension in water (with 1:1 molar ratio of 1 N HCl) once-daily (qd) or twice-daily (bid) at 10, 25, 50, or 100-mg/kg for 19 days. During the dosing period of 14-19 days, tumor weights are determined twice-weekly and body weights are recorded daily.

### U-87 MG Human Glioblastoma Model

U-87 MG human glioblastoma cells are cultured in vitro in DMEM (Mediatech) supplemented with 10% Fetal Bovine Serum (Hyclone), Penicillin-Streptomycin and non-essential amino acids at 37° C. in a humidified 5% CO₂ atmosphere. On day 0, cells are harvested by trypsinization and 2×10⁶ cells (passage 5, 96% viability) in 0.1 mL of ice-cold Hank's balanced salt solution are implanted intradermally into the hindflank of 5-8 week old female nude mice. A transponder is implanted in each mouse for identification, and animals are monitored daily for clinical symptoms and survival. Body weights are recorded daily.

### A549 Human Lung Carcinoma Model

A549 human lung carcinoma cells are cultured in vitro in DMEM (Mediatech) supplemented with 10% Fetal Bovine Serum (Hyclone), Penicillin-Streptomycin and non-essential amino acids at 37° C. in a humidified 5% CO₂ atmosphere. On day 0, cells are harvested by trypsinization and 10×10⁶ cells (passage 12, 99% viability) in 0.1 mL of ice-cold Hank's balanced salt solution are implanted intradermally into the hindflank of 5-8 week old female nude mice. A transponder is implanted in each mouse for identification, and animals are monitored daily for clinical symptoms and survival. Body weights are recorded daily.

### A2058 Human Melanoma Model

A2058 human melanoma cells are cultured in vitro in DMEM (Mediatech) supplemented with 10% Fetal Bovine Serum (Hyclone), Penicillin-Streptomycin and non-essential amino acids at 37° C. in a humidified, 5% CO₂ atmosphere. On day 0, cells are harvested by trypsinization and 3×10⁶ cells (passage 3, 95% viability) in 0.1 mL ice-cold Hank's balanced salt solution are implanted intradermally in the hind-flank of 5-8 week old female athymic nude mice. A transponder is implanted in each mouse for identification, and animals are monitored daily for clinical symptoms and survival. Body weights are recorded daily.

### WM-266-4 Human Melanoma Model

WM-266-4 human melanoma cells are cultured in vitro in DMEM (Mediatech) supplemented with 10% Fetal Bovine Serum (Hyclone), Penicillin-Streptomycin and non-essential amino acids at 37° C. in a humidified, 5% CO₂ atmosphere. On day 0, cells are harvested by trypsinization and 3×10⁶ cells (passage 5, 99% viability) in 0.1 mL ice-cold Hank's balanced salt solution are implanted intradermally in the hind-flank of 5-8 week old female athymic nude mice. A transponder is implanted in each mouse for identification, and animals are monitored daily for clinical symptoms and survival. Body weights are recorded daily.

Tumor weight (TW) in the above models is determined by measuring perpendicular diameters with a caliper, using the following formula:

tumor weight (mg)=[tumor volume=length (mm)×width² (mm²)]/2

These data were recorded and plotted on a tumor weight vs. days post-implantation line graph and presented graphically as an indication of tumor growth rates. Percent inhibition of tumor growth (TGI) is determined with the following formula:

$$[ 1 - ( \frac{( {X}_{f} - {X}_{0} )}{( {Y}_{f} - {X}_{0} )} ) ] * 100$$

where X₀=average TW of all tumors on group day

X_{f}=TW of treated group on Day f

Y_{f}=TW of vehicle control group on Day f

If tumors regress below their starting sizes, then the percent tumor regression is determined with the following formula:

$$( \frac{{X}_{0} - {X}_{f}}{{X}_{0}} ) * 100$$

Tumor size is calculated individually for each tumor to obtain a mean±SEM value for each experimental group. Statistical significance is determined using the 2-tailed Student's t-test (significance defined as P<0.05).

The foregoing invention has been described in some detail by way of illustration and example, for purposes of clarity and understanding. The invention has been described with reference to various specific embodiments and techniques. However, it should be understood that many variations and modifications may be made while remaining within the spirit and scope of the invention. it will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

1. A compound of formula I 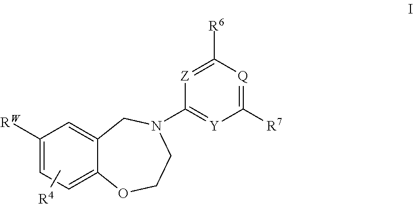
or a single isomer or mixture of stereoisomers thereof, optionally as a pharmaceutically acceptable salt thereof, wherein
R^{W} is 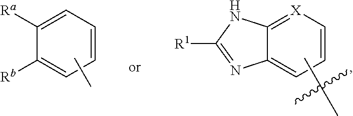
wherein:
R^{a} and le are each independently —CO(C₁-C₆)alkyl, —CO₂(C₁-C₆)alkyl, —SO₂—(C₁-C₆)alkyl, —NHSO₂—(C₁-C₆)alkyl, O—(C₁-C₆)alkyl, O—(C₁-C₆)haloalkyl, O—(C₁-C₆)alkylene-OR′″, wherein R′″ is H or (C₁-C₆)alkyl
R¹ is H, (C₁-C₆)alkyl), NH₂, NH(C₁-C₆)alkyl, halo(C₁-C₆)alkyl, (C₃-C₇)-cycloalkyl, NHCO₂(C₁-C₆)alkyl, or N((C₁-C₆)alkyl)₂;
X is N or CH;
R⁴ is H or halo;
Q is N, C—H, or C—(C₁-C₆)alkyl, C—CN, or C—CF₃;
R⁶ is H, (C₁-C₆)alkyl, halo(C₁-C₆)alkyl, (C₁-C₆)alkylene-NH₂, (C₁-C₆)alkylene-NH(C₁-C₆)alkyl, (C₁-C₆)alkylene-NH(C₁-C₆)haloalkyl, (C₁-C₆)alkylene-N(C₁-C₆)alkyl)₂, NH₂, NH(C₁-C₆)alkyl, hydroxyalkyl, (C₁-C₆)alkylene-O(C₁-C₆)alkyl, NH(C₁-C₆)alkyleneNH₂, NH(C₁-C₆)alkylene-cycloalkyl, —NH(C₁-C₆)alkylene-heterocycloalkyl, N((C₁-C₆)alkyl)₂, (C₁-C₆)alkylene-NHSO₂—(C₁-C₆)alkyl, (C₁-C₆)alkylene-NH(C═O)—(C₁-C₆)alkyl, —(C═O)—NH₂, —(C═O)—(C₁-C₆)alkyl, —(C═O)—NH(C₁-C₆)alkyl, —(C═O)—N(C₁-C₆)alkyl))₂, —NHSO₂—(C₁-C₆)alkyl, —S(O)—(C₁-C₆)alkyl, —SO₂—(C₁-C₆)alkyl, —SO₂NH₂, —SO₂NH(C₁-C₆)alkyl, —SO₂N((C₁-C₆)alkyl)₂, —CN, (C₄-C₇)heterocycloalkyl, (C₁-C₆)alkylene-(C₃-C₇)heterocycloalkyl, nitro, (C₁-C₆)alkylene-CN, NH(C₁-C₆)alkylene-NH(C₁-C₆)alkyl, NH(C₁-C₆)alkylene-N((C₁-C₆)alkyl)₂, or (C₁-C₆)alkylene-OC(O)—(C₁-C₆)alkyl, where any alkylene in R⁶ is optionally substituted with 1, 2, or 3 groups which are independently halo or hydroxy, and wherein when any alkylene is —CH₂—, then one of the hydrogens of the —CH₂— can optionally be replaced by CF₃, CHF₂, or CH₂F;
R⁷ is H, halo, —NH₂, nitro (C₁-C₆)alkyl, (C₁-C₆)alkoxy, —CF₃, halo(C₁-C₆)alkyl, (C₁-C₆)alkenyl, NH(C₁-C₆)alkyl, or N((C₁-C₆)alkyl)₂;
Y is N or C—R⁸, wherein R⁸ is H, halo, (C₁-C₆)alkyl, —CF₃, NH₂, NH(C₁-C₆)alkyl, N((C₁-C₆)alkyl)₂, (C₂-C₆)alkenyl, (C₁-C₆)alkylene-O(C₁-C₆)alkyl, hydroxyalkyl, (C₁-C₆)alkylene-CO₂(C₁-C₆)alkyl, (C₁-C₆)alkylene-CO₂H, phenyl, —CF₃, halo(C₁-C₆)alkyl, (C₃-C₇)cycloalkyl, (C₁-C₆)alkylene-(C₃-C₇)cycloalkyl, COH, CO₂H, —CO₂(C₁-C₆)alkyl, CN, (C₁-C₆)alkylene-CN, (C₁-C₆)alkylene-C≡C—H, (C₁-C₆)alkylene-C≡C—(C₁-C₆)alkyl, —C≡C—(C₁-C₆)alkyl, (C₁-C₆)alkylene-phenyl; where any phenyl in R⁸ is optionally substituted with 1, 2, or 3 groups which groups are independently halo or alkyl; or
R⁷ and R⁸, together with the atoms to which they are attached, can be joined together to form a 5, 6, or 7 membered saturated, partially unsaturated, or unsaturated ring, optionally containing up to two heteroatoms selected from N—H, N—(C₁-C₆)alkyl, O, SO, and SO₂ and where the ring formed by R⁷ and R⁸ is optionally substituted with one or two groups which are independently alkyl, alkoxy, or halo; and
Z is N or C—R⁹, wherein R⁹ is H, halo, or (C₁-C₆)alkyl; and
where at least one of Q and Z is N.

2. The compound of claim 1, wherein the compound is a compound of formula Ia or I-2.

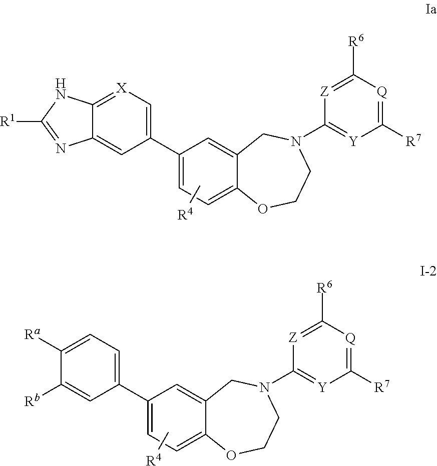

3. The compound of claim 2, wherein the compound is a compound of formula Ia.

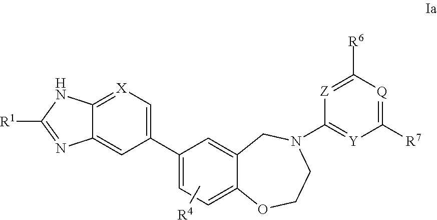

4. The compound of claim 3, wherein the compound is a compound of formula Ib.

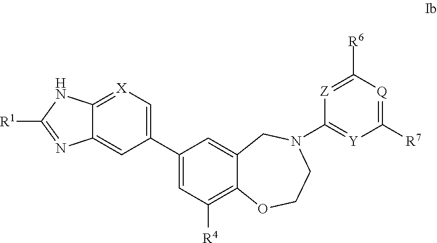

5. The compound of claim 4, wherein the compound is a compound of formula Ic-1 or Ic-2.

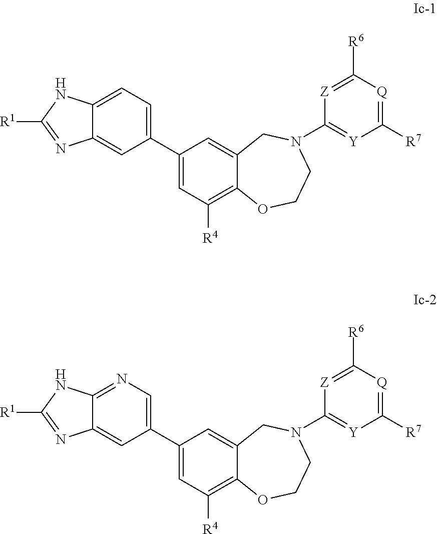

6. The compound of claim 2, wherein the compound is a compound of formula Id, wherein R⁴ is H or F.

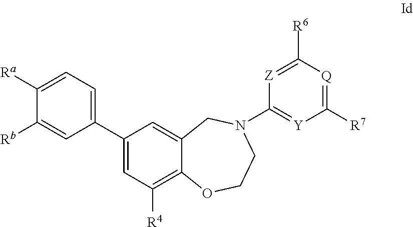

7. The compound of claim 2, wherein the compound is a compound of formula Ie.

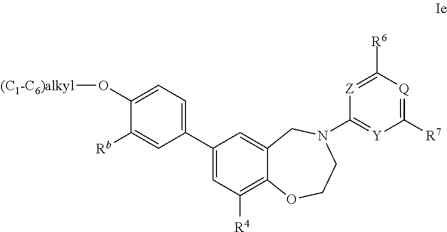

8. The compound of claim 7, wherein R⁴ is H.

9. The compounds of claim 8, wherein

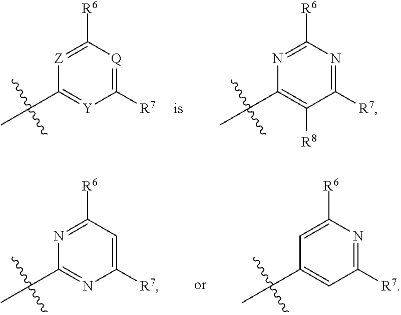

10. The compound of claim 9, wherein

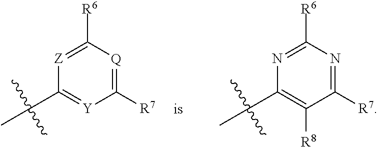

11. The compound of claim 8, wherein 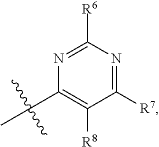
is selected from the group consisting of

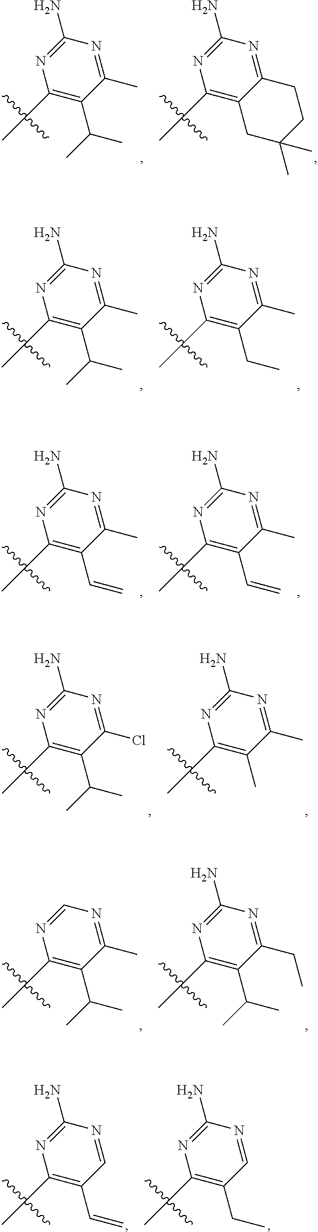

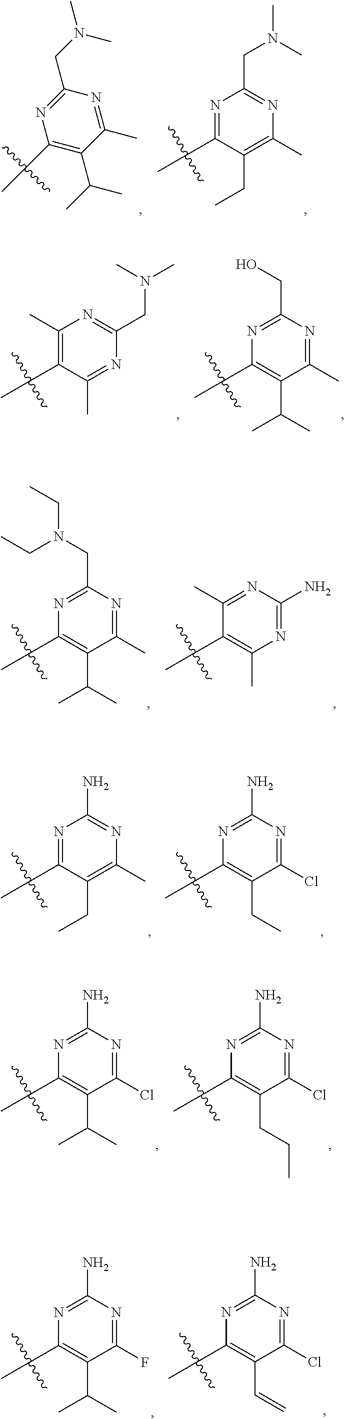

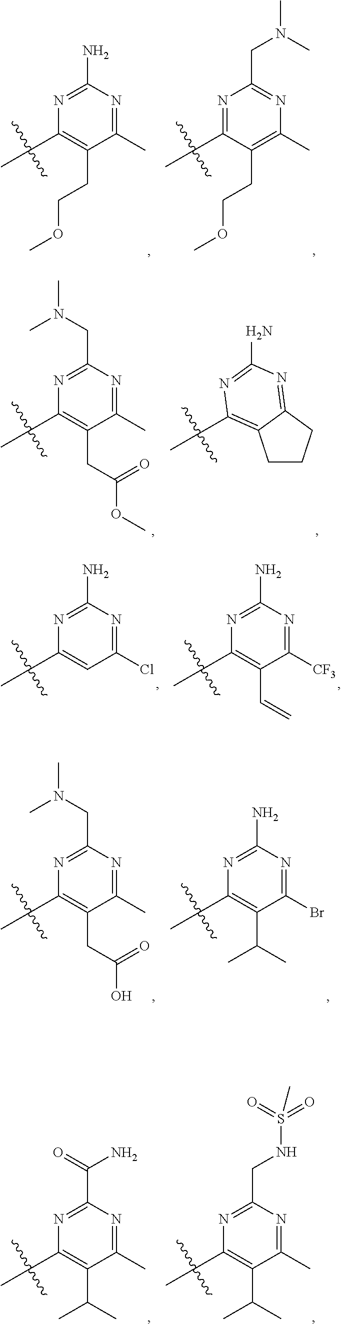

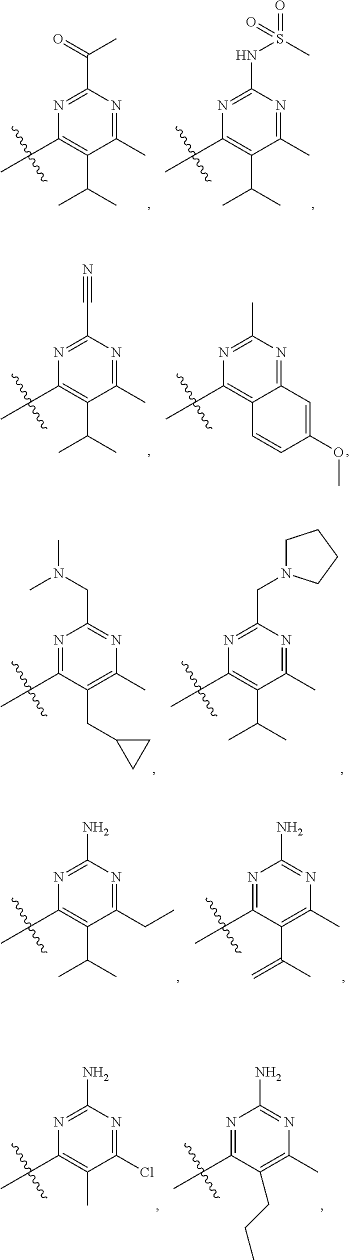

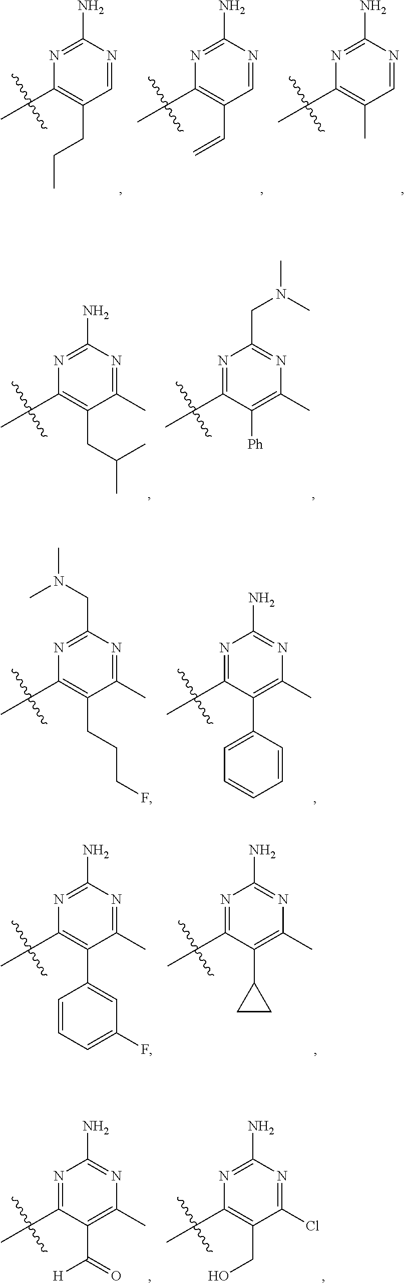

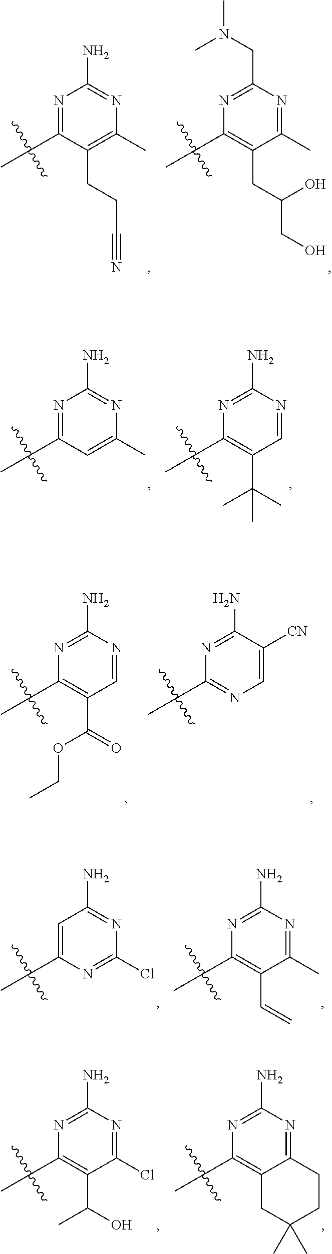

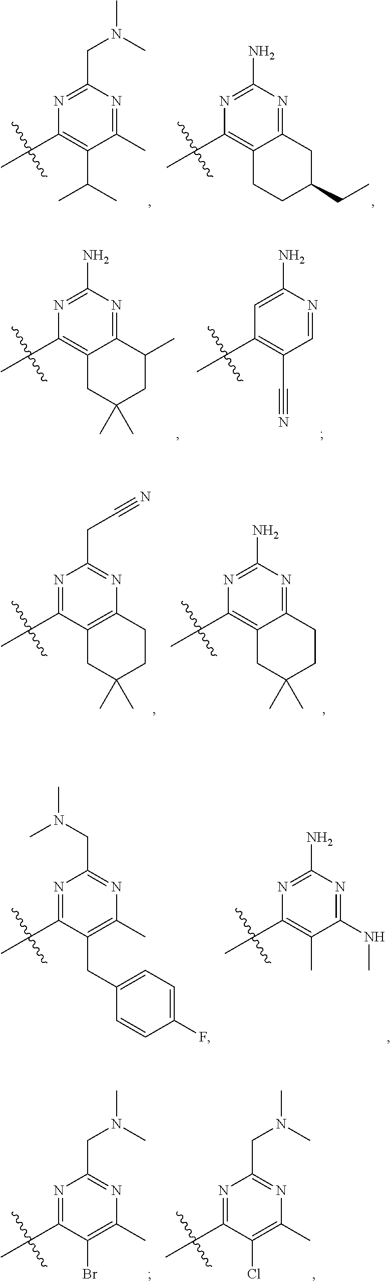

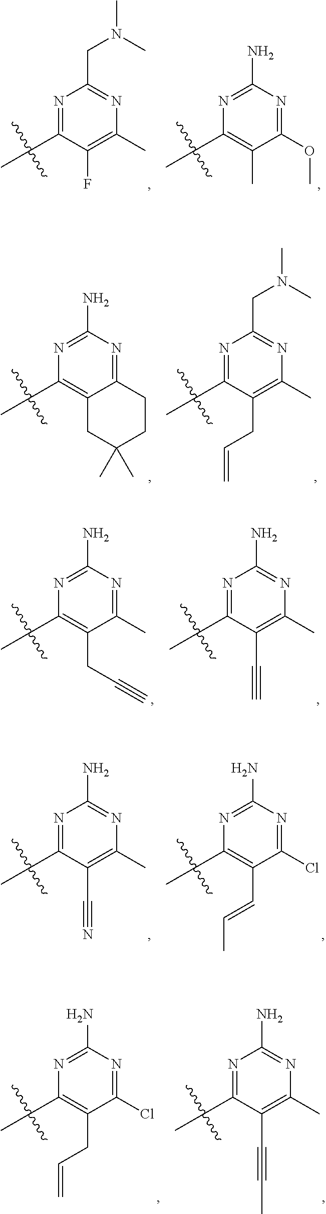

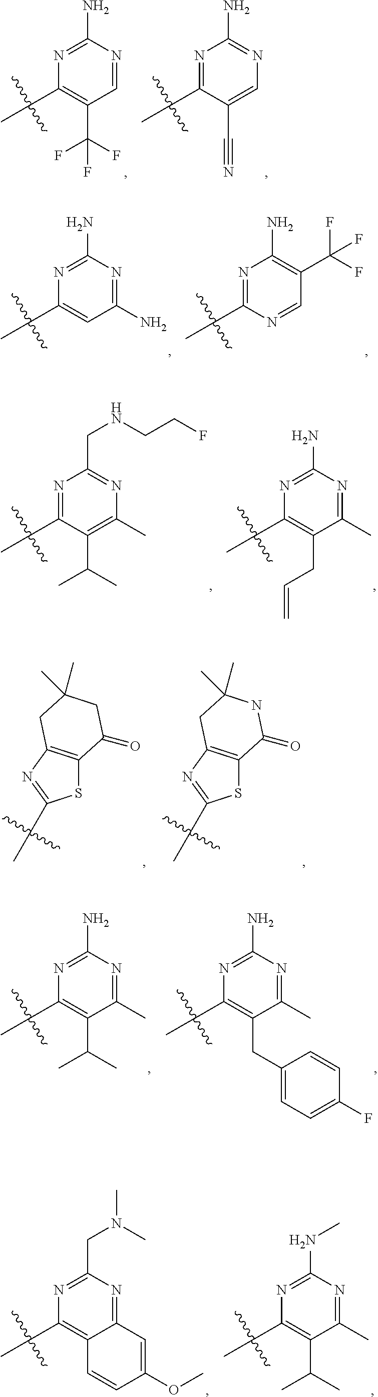

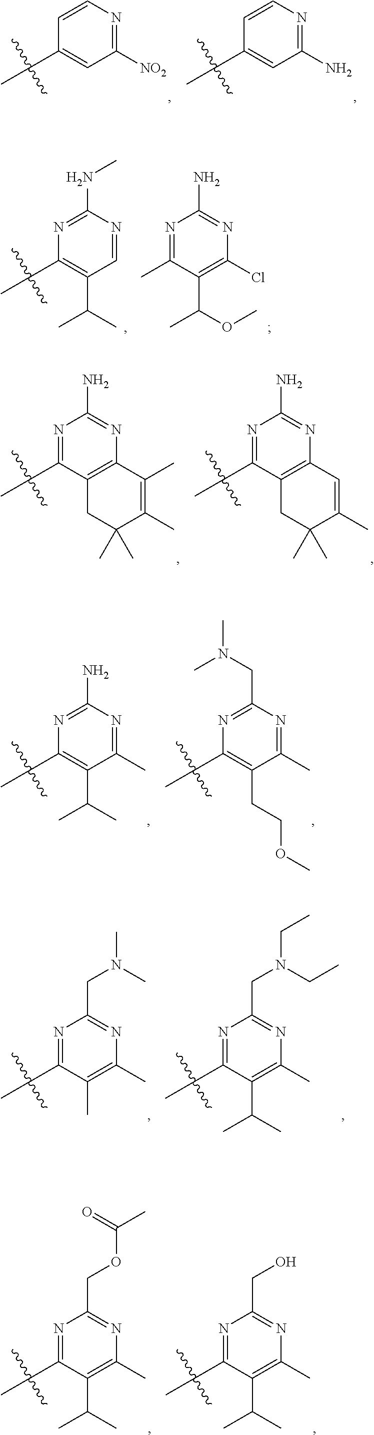

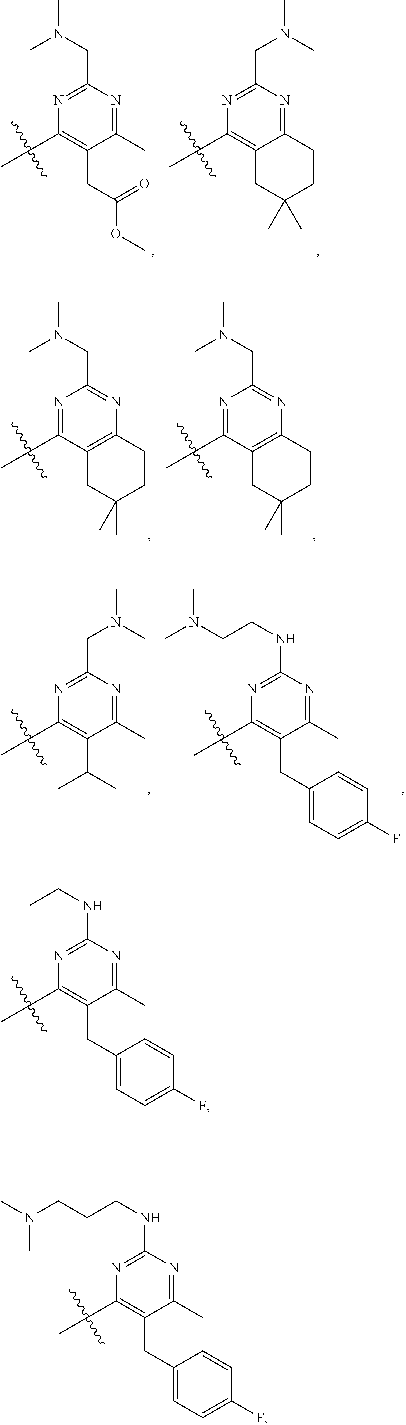

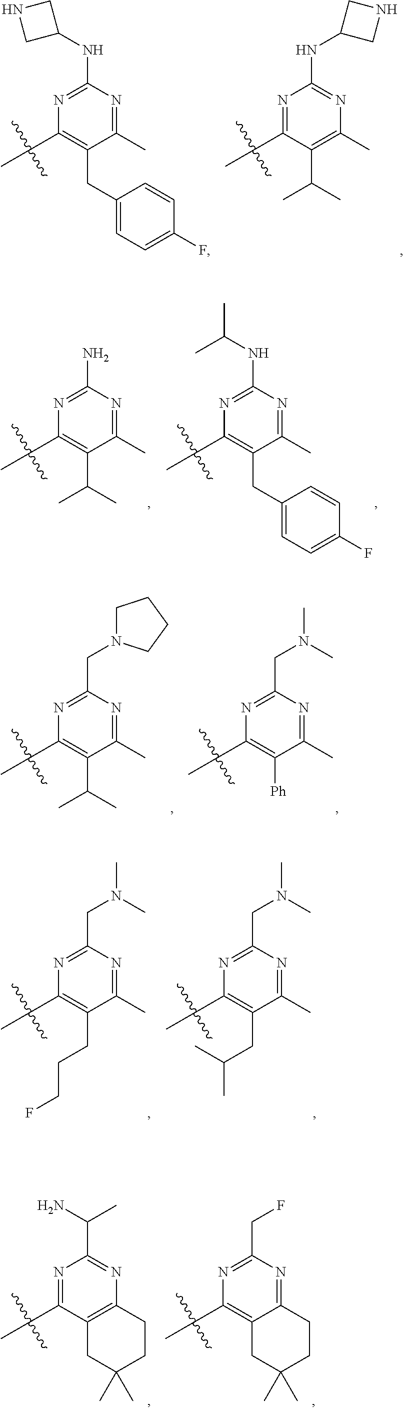

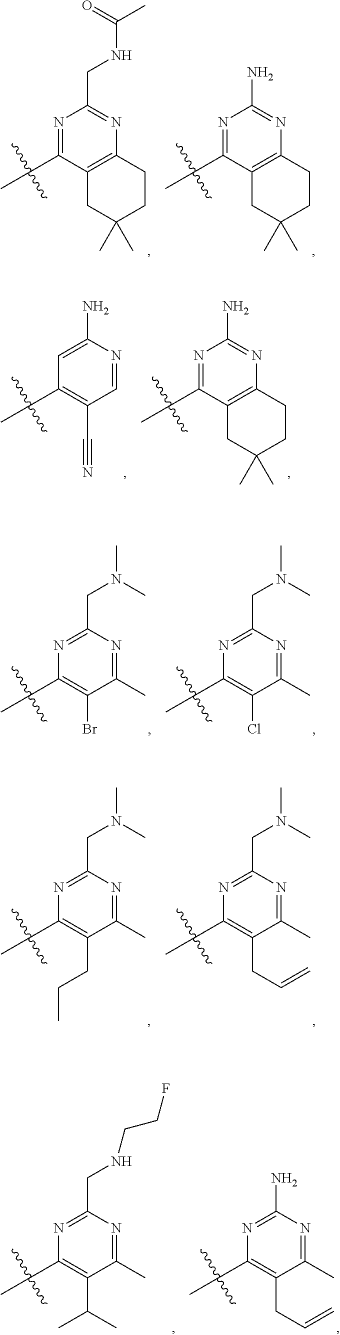

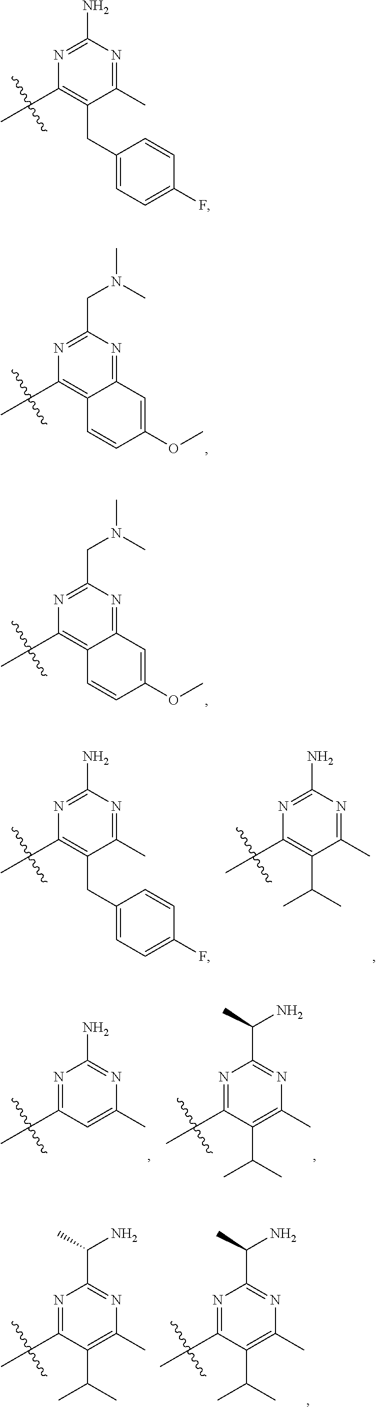

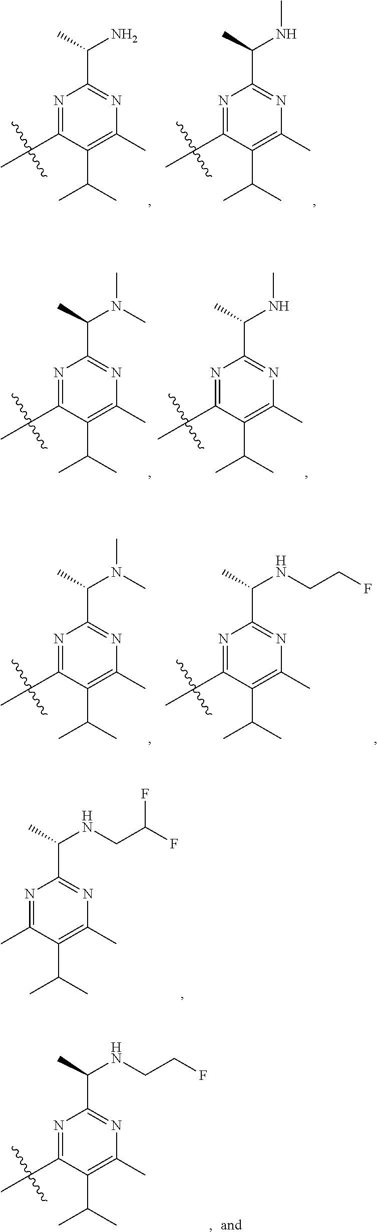

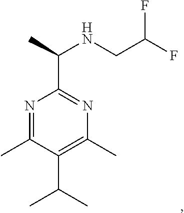 [SMILES: Cc1nc([C@@H](C)NCC(F)F)nc(C)c1C(C)C]

12. The compound of claim 11, wherein 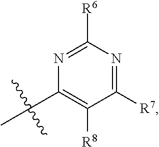
is selected from the group consisting of

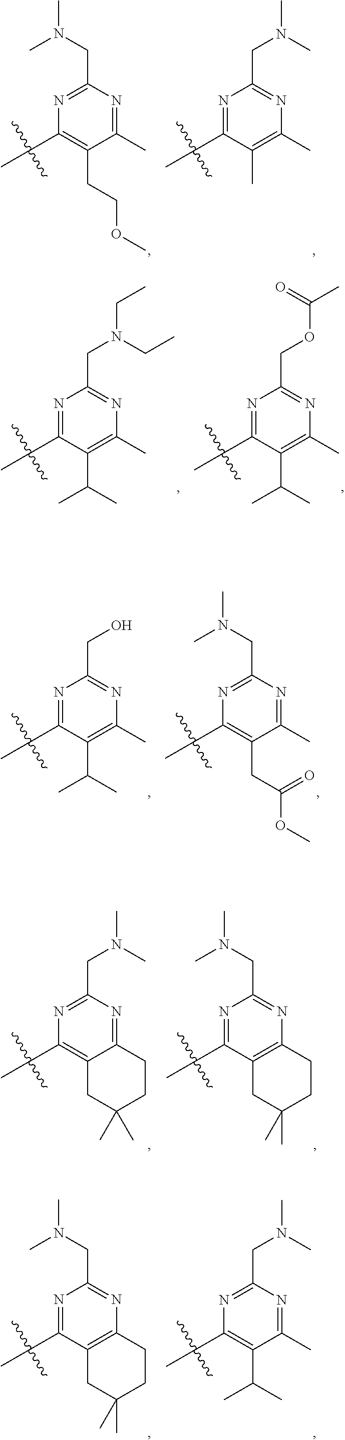

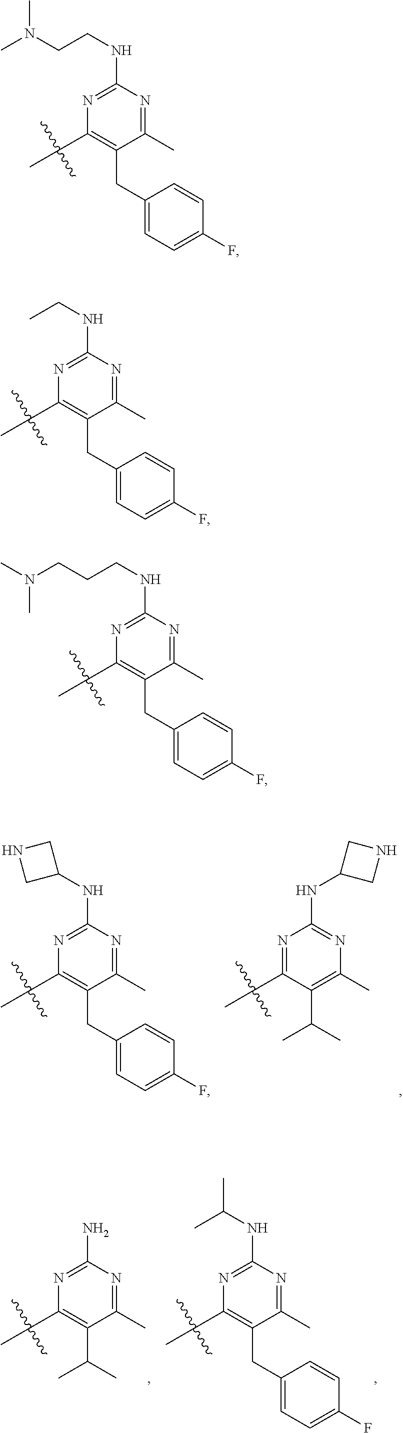

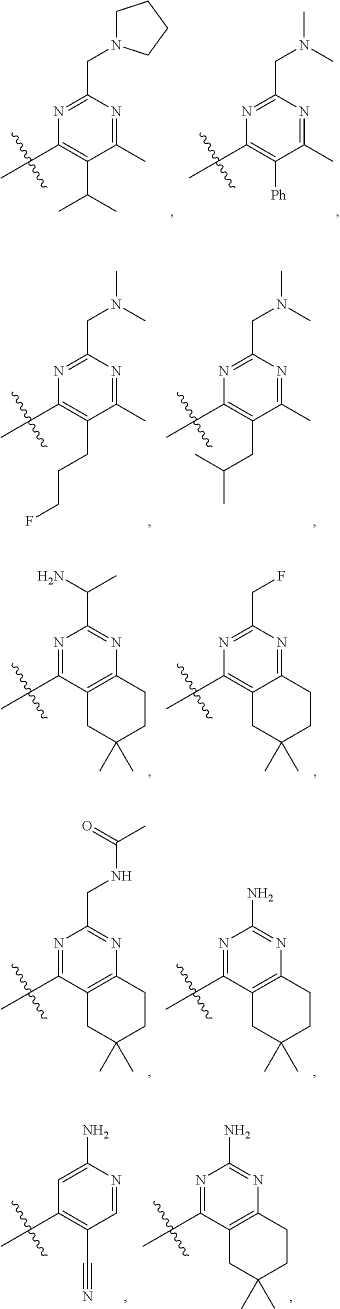

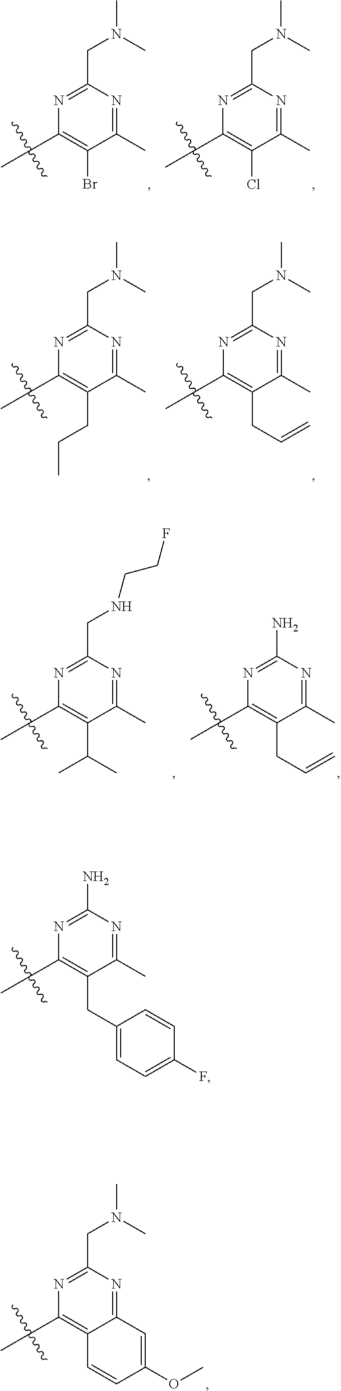

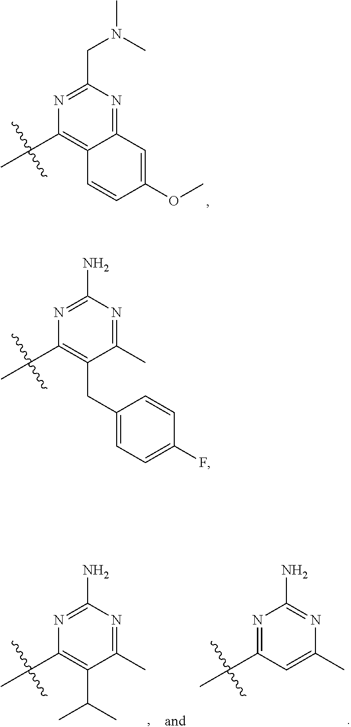

13. The compound of claim 1, which is:
N,N-dimethyl-1-{4-methyl-6-[7-(2-methyl-1H-benzimidazol-6-yl)-2,3-dihydro-1,4-benzoxazepin-4(5H)-yl]-5-[2-(methyloxy)ethyl]pyrimidin-2-yl}methanamine;
1-{4,5-dimethyl-6-[7-(2-methyl-1H-benzimidazol-6-yl)-2,3-dihydro-1,4-benzoxazepin-4(5H)-yl]pyrimidin-2-yl}-N,N-dimethylmethanamine;
N-ethyl-N-({4-methyl-6-[7-(2-methyl-1-benzimidazol-5-yl)-2,3-dihydro-1,4-benzoxazepin-4(5H)-yl]-5-(1-methylethyl)pyrimidin-2-yl}methyl)ethanamine;
{4-methyl-6-[7-(2-methyl-1H-benzimidazol-5-yl)-2,3-dihydro-1,4-benzoxazepin-4(5H)-yl]-5-(1-methylethyl)pyrimidin-2-yl}methyl acetate;
{4-methyl-6-[7-(2-methyl-1H-benzimidazol-5-yl)-2,3-dihydro-1,4-benzoxazepin-4(5H)-yl]-5-(1-methylethyl)pyrimidin-2-yl}methanol;
methyl {2-[(dimethylamino)methyl]-4-methyl-6-[7-(2-methyl-1H-benzimidazol-5-yl)-2,3-dihydro-1,4-benzoxazepin-4(5H)-yl]pyrimidin-5-yl}acetate;
1-(6,6-dimethyl-4-{7-[4-(methyloxy)-3-{[2-(methyloxy)ethyl]oxy}phenyl]-2,3-dihydro-1,4-benzoxazepin-4(5H)-yl}-5,6,7,8-tetrahydroquinazolin-2-yl)-N,N-dimethylmethanamine;
1-{4-[7-{3-[(difluoromethyl)oxy]-4-(methyloxy)phenyl}-2,3-dihydro-1,4-benzoxazepin-4(5H)-yl]-6,6-dimethyl-5,6,7,8-tetrahydroquinazolin-2-yl}-N,N-dimethylmethanamine; 1-(6,6-dimethyl-4-{7-[4-(methyloxy)-3-(methylsulfonyl)phenyl]-2,3-dihydro-1,4-benzoxazepin-4(5H)-yl}-5,6,7,8-tetrahydroquinazolin-2-yl)-N,N-dimethylmethanamine;
N-[5-(4-{2-[(dimethylamino)methyl]-6-methyl-5-(1-methylethyl)pyrimidin-4-yl}-2,3,4,5-tetrahydro-1,4-benzoxazepin-7-yl)-2-(methyloxy)phenyl]methanesulfonamide;
N′-{5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-yl}-N,N-dimethylethane-1,2-diamine;
N-ethyl-5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-amine;
N′-{5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-yl}-N,N-dimethylpropane-1,3-diamine;
N-azetidin-3-yl-5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-amine;
N-azetidin-3-yl-4-methyl-6-[7-(2-methyl-1H-benzimidazol-5-yl)-2,3-dihydro-1,4-benzoxazepin-4(5H)-yl]-5-(1-methylethyl)pyrimidin-2-amine;
N-[5-{4-[2-amino-6-methyl-5-(1-methylethyl)pyrimidin-4-yl]-2,3,4,5-tetrahydro-1,4-benzoxazepin-7-yl}-2-(methyloxy)phenyl]methanesulfonamide;
methyl (5-{4-[2-amino-6-methyl-5-(1-methylethyl)pyrimidin-4-yl]-2,3,4,5-tetrahydro-1,4-benzoxazepin-7-yl}-1H-benzimidazol-2-yl)carbamate;
1-[5-(4-{2-[(dimethylamino)methyl]-6,6-dimethyl-5,6,7,8-tetrahydroquinazolin-4-yl}-2,3,4,5-tetrahydro-1,4-benzoxazepin-7-yl)-2-(methyloxy)phenyl]ethanone;
5-[(4-fluorophenyl)methyl]-4-methyl-6-[7-(2-methyl-1H-benzimidazol-5-yl)-2,3-dihydro-1,4-benzoxazepin-4(5H)-yl]-N-(1-methylethyl)pyrimidin-2-amine;
7-(2-methyl-1H-benzimidazol-5-yl)-4-[6-methyl-5-(1-methylethyl)-2-(pyrrolidin-1-ylmethyl)pyrimidin-4-yl]-2,3,4,5-tetrahydro-1,4-benzoxazepine;
N,N-dimethyl-1-{4-methyl-6-[7-(2-methyl-1H-benzimidazol-5-yl)-2,3-dihydro-1,4-benzoxazepin-4(5H)-yl]-5-phenylpyrimidin-2-yl}methanamine;
1-{5-(3-fluoropropyl)-4-methyl-6-[7-(2-methyl-1H-benzimidazol-5-yl)-2,3-dihydro-1,4-benzoxazepin-4(5H)-yl]pyrimidin-2-yl}-N,N-dimethylmethanamine
N,N-dimethyl-1-{4-methyl-6-[7-(2-methyl-1H-benzimidazol-5-yl)-2,3-dihydro-1,4-benzoxazepin-4(5H)-yl]-5-(2-methylpropyl)pyrimidin-2-yl}methanamine;
1-{6,6-dimethyl-4-[7-(2-methyl-1H-benzimidazol-5-yl)-2,3-dihydro-1,4-benzoxazepin-4(5H)-yl]-5,6,7,8-tetrahydroquinazolin-2-yl}ethanamine;
4-[2-(fluoromethyl)-6,6-dimethyl-5,6,7,8-tetrahydroquinazolin-4-yl]-7-(2-methyl-1H-benzimidazol-5-yl)-2,3,4,5-tetrahydro-1,4-benzoxazepine;
N-({6,6-dimethyl-4-[7-(2-methyl-1H-benzimidazol-5-yl)-2,3-dihydro-1,4-benzoxazepin-4(5H)-yl]-5,6,7,8-tetrahydroquinazolin-2-yl}methyl)acetamide;
4-[7-(1H-benzimidazol-5-yl)-2,3-dihydro-1,4-benzoxazepin-4(5H)-yl]-6,6-dimethyl-5,6,7,8-tetrahydroquinazolin-2-amine;
6-amino-4-[7-(2-methyl-1H-benzimidazol-5-yl)-2,3-dihydro-1,4-benzoxazepin-4(5H)-yl]pyridine-3-carbonitrile;
6,6-dimethyl-4-[7-(2-methyl-1H-benzimidazol-5-yl)-2,3-dihydro-1,4-benzoxazepin-4(5H)-yl]-5,6,7,8-tetrahydroquinazolin-2-amine;
1-{5-bromo-4-methyl-6-[7-(2-methyl-1H-benzimidazol-5-yl)-2,3-dihydro-1,4-benzoxazepin-4(5H)-yl]pyrimidin-2-yl}-N,N-dimethylmethanamine;
1-{5-chloro-4-methyl-6-[7-(2-methyl-1H-benzimidazol-5-yl)-2,3-dihydro-1,4-benzoxazepin-4(5H)-yl]pyrimidin-2-yl}-N,N-dimethylmethanamine;
N,N-dimethyl-1-{4-methyl-6-[7-(2-methyl-1H-benzimidazol-6-yl)-2,3-dihydro-1,4-benzoxazepin-4(5H)-yl]-5-propylpyrimidin-2-yl}methanamine;
N,N-dimethyl-1-{4-methyl-6-[7-(2-methyl-1H-benzimidazol-6-yl)-2,3-dihydro-1,4-benzoxazepin-4(5H)-yl]-5-prop-2-en-1-ylpyrimidin-2-yl}methanamine;
2-fluoro-N-({4-methyl-6-[7-(2-methyl-1H-benzimidazol-5-yl)-2,3-dihydro-1,4-benzoxazepin-4(5H)-yl]-5-(1-methylethyl)pyrimidin-2-yl}methyl)ethanamine;
4-methyl-6-[7-(2-methyl-1H-benzimidazol-5-yl)-2,3-dihydro-1,4-benzoxazepin-4(5H)-yl]-5-prop-2-en-1-ylpyrimidin-2-amine;
4-[7-(1H-benzimidazol-5-yl)-2,3-dihydro-1,4-benzoxazepin-4(5H)-yl]-5-[(4-fluorophenyl)methyl]-6-methylpyrimidin-2-amine;
1-{4-[7-(1H-benzimidazol-5-yl)-2,3-dihydro-1,4-benzoxazepin-4(5H)-yl]-7-(methyloxy)quinazolin-2-yl}-N,N-dimethylmethanamine;
N,N-dimethyl-1-{4-[7-(2-methyl-1H-benzimidazol-5-yl)-2,3-dihydro-1,4-benzoxazepin-4(5H)-yl]-7-(methyloxy)quinazolin-2-yl}methanamine;
5-[(4-fluorophenyl)methyl]-4-methyl-6-[7-(2-methyl-1H-benzimidazol-5-yl)-2,3-dihydro-1,4-benzoxazepin-4(5H)-yl]pyrimidin-2-amine;
4-[7-(1H-benzimidazol-5-yl)-2,3-dihydro-1,4-benzoxazepin-4(5H)-yl]-6-methyl-5-(1-methylethyl)pyrimidin-2-amine;
4-methyl-6-[7-(2-methyl-1H-benzimidazol-5-yl)-2,3-dihydro-1,4-benzoxazepin-4(5H)-yl]-5-(1-methylethyl)pyrimidin-2-amine;
5-{4-[2-amino-6-methyl-5-(1-methylethyl)pyrimidin-4-yl]-2,3,4,5-tetrahydro-1,4-benzoxazepin-7-yl}-N-ethyl-1H-benzimidazol-2-amine;
4-[7-(1H-benzimidazol-5-yl)-2,3-dihydro-1,4-benzoxazepin-4(5H)-yl]-6-methylpyrimidin-2-amine;
4-[9-fluoro-7-(2-methyl-1H-benzimidazol-6-yl)-2,3-dihydro-1,4-benzoxazepin-4(5H)-yl]-6-methyl-5-(1-methylethyl)pyrimidin-2-amine;
6,6-dimethyl-4-[7-(2-methyl-1H-imidazo[4,5-b]pyridin-6-yl)-2,3-dihydro-1,4-benzoxazepin-4(5H)-yl]-5,6,7,8-tetrahydroquinazolin-2-amine;
4-[7-(3H-imidazo[4,5-b]pyridin-6-yl)-2,3-dihydro-1,4-benzoxazepin-4(5H)-yl]-6,6-dimethyl-5,6,7,8-tetrahydroquinazolin-2-amine;
6-{4-[2-amino-6-methyl-5-(1-methylethyl)pyrimidin-4-yl]-2,3,4,5-tetrahydro-1,4-benzoxazepin-7-yl}-3H-imidazo[4,5-b]pyridin-2-amine;
5-[(4-fluorophenyl)methyl]-4-[7-(3H-imidazo[4,5-b]pyridin-6-yl)-2,3-dihydro-1,4-benzoxazepin-4(5H)-yl]-6-methylpyrimidin-2-amine;
5-[(4-fluorophenyl)methyl]-4-methyl-6-[7-(2-methyl-3H-imidazo[4,5-b]pyridin-6-yl)-2,3-dihydro-1,4-benzoxazepin-4(5H)-yl]pyrimidin-2-amine;
1-{4-[7-(3H-imidazo[4,5-b]pyridin-6-yl)-2,3-dihydro-1,4-benzoxazepin-4(5H)-yl]-7-(methyloxy)quinazolin-2-yl}-N,N-dimethylmethanamine;
4-methyl-5-(1-methylethyl)-6-[7-(2-methyl-3H-imidazo[4,5-b]pyridin-6-yl)-2,3-dihydro-1,4-benzoxazepin-4(5H)-yl]pyrimidin-2-amine;
4-[7-(3H-imidazo[4,5-b]pyridin-6-yl)-2,3-dihydro-1,4-benzoxazepin-4(5H)-yl]-6-methyl-5-(1-methylethyl)pyrimidine-2-amine;
optionally as a pharmaceutically acceptable salt thereof.

14. A pharmaceutical composition which comprises a compound of claim 1, optionally as pharmaceutically acceptable salt thereof, and a pharmaceutically acceptable carrier, excipient, or diluent.

15. A method for treating a disease, disorder, or syndrome which method comprises administering to a patient a therapeutically effective amount of a compound of claim 1, optionally as a pharmaceutically acceptable salt thereof, or administering to a patient a pharmaceutical composition comprising a therapeutically effective amount of a compound of claim 1, optionally as a pharmaceutically acceptable salt thereof, and a pharmaceutically acceptable carrier, excipient, or diluent.

16. The method of claim 15, wherein the disease is cancer.

17. The method of claim 16, where the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.


SEQUENCE LISTING (8 sequences)

SEQ ID NO: 1 - protein, 1068 aa, Homo sapiens
  MISC_FEATURE: Human phosphatidylinositol 3-kinase catalytic subunit alpha polypeptide (PIK3CA) encoded by mRNA polynucleotide sequence ID NCBI Accession Reference No NM_006218; GI54792081 (3724 nucleotides)
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 2 - DNA, 3724 bp, Homo sapiens
  misc_feature: Human phosphatidylinositol 3-kinase catalytic subunit alpha mRNA polynucleotide sequence ID NCBI Accession Reference No NM_006218; GI54792081 (3724 nucleotides)
tctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaaaaaaaaaaaaaa

SEQ ID NO: 3 - DNA, 23 bp, Artificial Sequence
  human exon 9 forward primer
gggaaaaatatgacaaagaaagc

SEQ ID NO: 4 - DNA, 22 bp, Artificial Sequence
  human exon 9 reverse primer
ctgagatcagccaaattcagtt

SEQ ID NO: 5 - DNA, 27 bp, Artificial Sequence
  human exon 9 sequencing primer
tagctagagacaatgaattaagggaaa

SEQ ID NO: 6 - DNA, 20 bp, Artificial Sequence
  human exon 20 forward primer
ctcaatgatgcttggctctg

SEQ ID NO: 7 - DNA, 21 bp, Artificial Sequence
  human exon 20 reverse primers
tggaatccagagtgagctttc

SEQ ID NO: 8 - DNA, 22 bp, Artificial Sequence
  human exon 20 sequencing primer
ttgatgacattgcatacattcg